(19) United States
(12) Patent Application Publication
(10) Pub. No.: US 2004/0198651 A1
(43) Pub. Date: Oct. 7, 2004

(54) Secreted proteins

(76) Inventors: Aaron A. Klammer, Boulder, CO (US); April JA Hafalia, Daly City, CA (US); Brendan M Duggan, Sunnyvale, CA (US); Bridget A Warren, San Marcos, CA (US); Brooke M Emerling, Chicago, IL (US); Catherine M Tribouley, San Francisco, CA (US); Chandra S Arvizu, San Diego, CA (US); Cynthia D Honchell, San Carlos, CA (US); Danniel B Nguyen, San Jose, CA (US); Deborah A Kallick, Galveston, TX (US); Henry Yue, Sunnyvale, CA (US); Janice K Au-Young, Brisbane, CA (US); Jayalaxmi Ramkumar, Fremont, CA (US); Joana X. Li, Millbrae, CA (US); Kavitha Thangavelu, Sunnyvale, CA (US); Kimberly J Gietzen, San Jose, CA (US); Li Ding, Creve Coeur, MO (US); Mariah R Baughn, Los Angeles, CA (US); Monique G Yao, Mountain View, CA (US); Narinder K Chawla, Union City, CA (US); Patricia M Mason, Morgan Hill, CA (US); Preeti G. Lal, Santa Clara, CA (US); Richard C Graul, San Francisco, CA (US); Roopa M Reddy, Fremont, CA (US); Shanya D Becha, San Francisco, CA (US); Stephanie K Kareht, Redwood City, CA (US); Thomas W Richardson, Redwood City, CA (US); Uyen K Tran, San Jose, CA (US); Vicki S Elliott, San Jose, CA (US); Y Tom Tang, San Jose, CA (US); Yalda Azimzai, Oakland, CA (US); Yan Lu, Mountain View, CA (US); Yuming Xu, Mountain View, CA (US)

Correspondence Address: FOLEY AND LARDNER, SUITE 500, 3000 K STREET NW, WASHINGTON, DC, 20007, US

(21) Appl. No.: 10/475,446

(22) PCT Filed: Apr. 19, 2002
(86) PCT No.: PCT/US02/12464

Related U.S. Application Data
(60) Provisional application No. 60/285,207, filed on Apr. 20, 2001.
(60) Provisional application No. 60/287,114, filed on Apr. 27, 2001.
(60) Provisional application No. 60/288,640, filed on May 3, 2001.
(60) Provisional application No. 60/290,516, filed on May 11, 2001.
(60) Provisional application No. 60/292,184, filed on May 18, 2001.
(60) Provisional application No. 60/343,553, filed on Dec. 21, 2001.
(60) Provisional application No. 60/357,002, filed on Feb. 13, 2002.
(60) Provisional application No. 60/358,279, filed on Feb. 20, 2002.
(60) Provisional application No. 60/366,041, filed on Mar. 19, 2002.

(51) Int. Cl. G01N 33/574; A61K 38/17; C07K 14/47; C12N 5/06

(52) U.S. Cl. 514/12; 530/350; 435/69.1; 435/320.1; 435/325; 536/23.5

(57) ABSTRACT

The invention provides human secreted proteins (SECP) and polynucleotides which identify and encode SECP. The invention also provides expression vectors, host cells, antibodies, agonists, and antagonists. The invention also provides methods for diagnosing, treating, or preventing disorders associated with aberrant expression of SECP.

### TECHNICAL FIELD

This invention relates to nucleic acid and amino acid sequences of secreted proteins and to the use of these sequences in the diagnosis, treatment, and prevention of liver, cell proliferative, autoimmune/inflammatory, cardiovascular, neurological, and deveopmental disorders, and in the assessment of the effects of exogenous compounds on the expression of nucleic acid and amino acid sequences of secreted proteins.

### BACKGROUND OF THE INVENTION

Protein transport and secretion are essential for cellular function. Protein transport is mediated by a signal peptide located at the amino terminus of the protein to be transported or secreted. The signal peptide is composed of about ten to twenty hydrophobic amino acids which target the nascent protein from the ribosome to a particular membrane bound compartment such as the endoplasmic reticulum (ER). Proteins targeted to the ER may either proceed through the secretory pathway or remain in any of the secretory organelles such as the ER, Golgi apparatus, or lysosomes. Proteins that transit through the secretory pathway are either secreted into the extracellular space or retained in the plasma membrane. Proteins that are retained in the plasma membrane contain one or more transmembrane domains, each comprised of about 20 hydrophobic amino acid residues. Secreted proteins are generally synthesized as inactive precursors that are activated by post-translational processing events during transit through the secretory pathway. Such events include glycosylation, proteolysis, and removal of the signal peptide by a signal peptidase. Other events that may occur during protein transport include chaperone-dependent unfolding and folding of the nascent protein and interaction of the protein with a receptor or pore complex. Examples of secreted proteins with amino terminal signal peptides are discussed below and include proteins with important roles in cell-to-cell signaling. Such proteins include transmembrane receptors and cell surface markers, extracellular matrix molecules, cytokines, hormones, growth and differentiation factors, enzymes, neuropeptides, and vasomediators. (Reviewed in Alberts, B. et al. (1994) Molecular Biology of The Cell, Garland Publishing, New York, N.Y., pp. 557-560, 582-592.)

Cell surface markers include cell surface antigens identified on leukocytic cells of the immune system. These antigens have been identified using systematic, monoclonal antibody (mAb)-based “shot gun” techniques. These techniques have resulted in the production of hundreds of mAbs directed against unknown cell surface leukocytic antigens. These antigens have been grouped into “clusters of differentiation” based on common immunocytochemical localization patterns in various differentiated and undifferentiated leukocytic cell types. Antigens in a given cluster are presumed to identify a single cell surface protein and are assigned a “cluster of differentiation” or “CD” designation. Some of the genes encoding proteins identified by CD antigens have been cloned and verified by standard molecular biology techniques. CD antigens have been characterized as both transmembrane proteins and cell surface proteins anchored to the plasma membrane via covalent attachment to fatty acid-containing glycolipids such as glycosylphosphatidylinositol (GPI). (Reviewed in Barclay, A. N. et al. (1995) The Leucocyte Antigen Facts Book, Academic Press, San Diego, Calif., pp. 17-20.)

Matrix proteins (MPs) are transmembrane and extracellular proteins which function in formation, growth, remodeling, and maintenance of tissues and as important mediators and regulators of the inflammatory response. The expression and balance of MPs may be perturbed by biochemical changes that result from congenital, epigenetic, or infectious diseases. In addition, MPs affect leukocyte migration, proliferation, differentiation, and activation in the immune response. MPs are frequently characterized by the presence of one or more domains which may include collagen-like domains, EGF-like domains, immunoglobulin-like domains, and fibronectin-like domains. In addition, MPs may be heavily glycosylated and may contain an Arginine-Glycine-Aspartate (RGD) tripeptide motif which may play a role in adhesive interactions. MPs include extracellular proteins such as fibronectin, collagen, galectin, vitronectin and its proteolytic derivative somatomedin B; and cell adhesion receptors such as cell adhesion molecules (CAMs), cadherins, and integrins. (Reviewed in Ayad, S. et al. (1994) The Extracellular Matrix Facts Book Academic Press, San Diego, Calif., pp.2-16; Ruoslahti, E. (1997) Kidney Int. 51:1413-1417; Sjaastad, M. D. and Nelson, W. J. (1997) BioEssays 19:47-55.)

Mucins are highly glycosylated glycoproteins that are the major structural component of the mucus gel. The physiological functions of mucins are cytoprotection, mechanical protection, maintenance of viscosity in secretions, and cellular recognition. MUC6 is a human gastric mucin that is also found in gall bladder, pancreas, seminal vesicles, and female reproductive tract (Toribara, N. W. et al. (1997) J. Biol. Chem. 272:16398-16403). The MUC6 gene has been mapped to human chromosome 11 (Toribara, N. W. et al. (1993) J. Biol. Chem 268:5879-5885). Hemomucin is a novel Drosophila surface mucin that may be involved in the induction of antibacterial effector molecules (Theopold, U. et al. (1996) J. Biol. Chem. 217:12708-12715).

Tuftelins are one of four different enamel matrix proteins that have been identified so far. The other three known enamel matrix proteins are the amelogenins, enamelin and ameloblastin. Assembly of the enamel extracellular matrix from these component proteins is believed to be critical in producing a matrix competent to undergo mineral replacement. (Paine C. T. et al. (1998) Connect Tissue Res.38:257-267). Tuftelin mRNA has been found to be expressed in human ameloblastoma tumor, a non-mineralized odontogenic tumor (Deutsch D. et al. (1998) Connect Tissue Res. 39:177-184)

Olfactomedin-related proteins are extracellular matrix, secreted glycoproteins with conserved C-terminal motifs. They are expressed in a wide variety of tissues and in broad range of species, from Caenorhabditis elegans to Homo sapiens. Olfactomedin-related proteins comprise a gene family with at least 5 family members in humans. One of the five, TIGR/myocilin protein, is expressed in the eye and is associated with the pathogenesis of glaucoma (Kulkarni, N. H. et al., (2000) Genet. Res. 76:41-50). Research by Yokoyama et al. (1996) found a 135-amino acid protein, termed AMY, having 96% sequence identity with rat neuronal olfactomedin-releated ER localized protein in a neuroblastoma cell line cDNA library, suggesting an essential role for AMY in nerve tissue (Yokoyama, M. et al., (1996) DNA Res.3:311-320). Neuron-specific olfactomedin-related glycoproteins isolated from rat brain cDNA libraries show strong sequence similarity with olfactomedin. This similarity is suggestive of a matrix-related function of these glycoproteins in neurons and neurosecretory cells (Danielson, P. E. et al., (1994) J. Neurosci. Res. 38:468-478).

Mac-2 binding protein is a 90-kD serum protein (90K) and another secreted glycoprotein, isolated from both the human breast carcinoma cell line SK-BR-3, and human breast milk. It specifically binds to a human macrophage-associated lectin, Mac-2. Structurally, the mature protein is 567 amino acids in length and is preceded by an 18-amino acid leader. There are 16 cysteines and seven potential N-linked glycosylation sites. The first 106 amino acids represent a domain very similar to an ancient protein superfamily defined by a macrophage scavenger receptor cysteine-rich domain (Koths, K. et al., (1993) J. Biol. Chem. 268:14245-14249). 90K is elevated in the serum of subpopulations of AIDS patients and is expressed at varying levels in primary tumor samples and tumor cell lines. Ullrich et al. (1994) have demonstrated that 90K stimulates host defense systems and can induce interleukin-2 secretion. This immune stimulation is proposed to be a result of oncogenic transformation, viral infection or pathogenic invasion (Ulrich; A., et al. (1994) J. Biol. Chem. 269:18401-18407).

Semaphorins are a large group of axonal guidance molecules consisting of at least 30 different members and are found in vertebrates, invertebrates, and even certain viruses. All semaphorins contain the sema domain which is approximately 500 amino acids in length. Neuropilin, a semaphorin receptor has been shown to promote neurite outgrowth in vitro. The extracellular region of neuropilins consists of three different domains: CUB, discoidin, and MAM domains. The CUB and the MAM motifs of neuropilin have been suggested as having roles in protein-protein interactions and are suggested to be involved in the binding of semaphorins through the sema and the C-terminal domains (reviewed in Raper, J. A. (2000) Curr. Opin. Neurobiol. 10:88-94). Plexins are neuronal cell surface molecules that mediate cell adhesion via a homophilic binding mechanism in the presence of calcium ions. Plexins have been shown to be expressed in the receptors and neurons of particular sensory systems (Ohta, K et al. (1995) Cell 14:1189-1199). There is evidence that suggests that some plexins function to control motor and CNS axon guidance in the developing nervous system Plexins, which themselves contain complete semaphorin domains, may be both the ancestors of classical semaphorins and binding partners for semaphorins (Winberg, M. L. et al (1998) Cell 95:903-916).

Human pregnancy-specific beta 1-glycoprotein (PSG) is a family of closely related glycoproteins of molecular weights of 72 KDa, 64 KDa, 62 KDa, and 54 KDa. Together with the carcinoembryonic antigen, they comprise a subfamily within the immunoglobulin superfamily (Plouzek C. A. and Chou J. Y. Endocrinology 129:950-958) Different subpopulations of PSG have been found to be produced by the trophoblasts of the human placenta, and the amnionic, and chorionic membranes (Plouzek C. A et al. (1993) Placenta 14:277-285).

Autocrine motility factor (AMF) is one of the motility cytokines regulating tumor cell migration, therefore identification of the signaling pathway coupled with it has critical importance. Autocrine motility factor receptor (AMFR) expression has been found to be associated with tumor progression in thymoma (Ohta Y. et al. (2000) Int J Oncol. 17:259-264). AMFR is a cell surface glycoprotein of molecular weight 78 KDa.

Hormones are secreted molecules that travel through the circulation and bind to specific receptors on the surface of, or within, target cells. Although they have diverse biochemical compositions and mechanisms of action, hormones can be grouped into two categories. One category includes small lipophilic hormones that diffuse through the plasma membrane of target cells, bind to cytosolic or nuclear receptors, and form a complex that alters gene expression. Examples of these molecules include retinoic acid, thyroxine, and the cholesterol-derived steroid hormones such as progesterone, estrogen, testosterone, cortisol, and aldosterone. The second category includes hydrophilic hormones that function by binding to cell surface receptors that transduce signals across the plasma membrane. Examples of such hormones include amino acid derivatives such as catecholamines (epinephrine, norepinephrine) and histamine, and peptide hormones such as glucagon, insulin, gastrin, secretin, cholecystokinin, adrenocorticotropic hormone, follicle stimulating hormone, luteinizing hormone, thyroid stimulating hormone, and vasopressin. (See, for example, Lodish et al. (1995) Molecular Cell Biology, Scientific American Books Inc., New York, N.Y., pp. 856-864.)

Pro-opiomelanocortin (POMC) is the precursor peptide of corticotropin (ACTH) a hormone synthesized by the anterior pituitary gland, which functions in the stimulation of the adrenal cortex. POMC is also the precursor peptide of the hormone, beta-lipotropin (beta-LPH),. Each hormone includes smaller peptides with distinct biological activities: alpha-melanotropin (alpha-MSH) and corticotropin-like intermediate lobe peptide (CLIP) are formed from ACTH; gamma-lipotropin (gamma-LPH) and beta-endorphin are peptide components of beta-LPH, while beta-MSH is contained within gamma-LPH. Adrenal insufficiency due to ACTH deficiency, resulting from a genetic mutation in exons 2 and 3 of POMC has defined a endocrine disorder characterized by early-onset obesity, adrenal insufficiency, and red hair pigmentation (Chretien, M. et al., (1979) Canad. J. Biochem. 57:1111-1121, Krude, H. et al., (1998) Nature Genet. 19:155-157, Online Mendelian Inheritance in Man, OMIM. Johns Hopkins University, Baltimore, Md. MIM Number: 176830: Aug. 1, 2000. World Wide Web URL: www.ncbi.nlm.nih.gov/omim/).

Growth and differentiation factors are secreted proteins which function in intercellular communication. Some factors require oligomerization or association with membrane proteins for activity. Complex interactions among these factors and their receptors trigger intracellular signal transduction pathways that stimulate or inhibit cell division, cell differentiation, cell signaling, and cell motility. Most growth and differentiation factors act on cells in their local environment (paracrine signaling). There are three broad classes of growth and differentiation factors. The first class includes the large polypeptide growth factors such as epidermal growth factor, fibroblast growth factor, transforming growth factor, insulin-like growth factor, and platelet-derived growth factor. The second class includes the hematopoietic growth factors such as the colony stimulating factors (CSFs). Hematopoietic growth factors stimulate the proliferation and differentiation of blood cells such as B-lymphocytes, T-lymphocytes, erythrocytes, platelets, eosinophils, basophils, neutrophils, macrophages, and their stem cell precursors. The third class includes small peptide factors such as bombesin, vasopressin, oxytocin, endothelin, transferrin, angiotensin II, vasoactive intestinal peptide, and bradykinin which function as hormones to regulate cellular functions other than proliferation.

Growth and differentiation factors play critical roles in neoplastic transformation of cells in vitro and in tumor progression in vivo. Inappropriate expression of growth factors by tumor cells may contribute to vascularization and metastasis of tumors. During hematopoiesis,,growth factor misregulation can result in anemias, leukemias, and lymphomas. Certain growth factors such as interferon are cytotoxic to tumor cells both in vivo and in vitro. Moreover, some growth factors and growth factor receptors are related both structurally and functionally to oncoproteins. In addition, growth factors affect transcriptional regulation of both proto-oncogenes and oncosuppressor genes. (Reviewed in Pimentel, E. (1994) Handbook of Growth Factors, CRC Press, Ann Arbor, Mich., pp. 1-9.)

The Slit protein, first identified in Drosophila, is critical in central nervous system midline formation and potentially in nervous tissue histogenesis and axonal pathfinding. Itoh et al. have identified mammalian homologues of the slit gene (human Slit-1, Slit-2, Slit-3 and rat Slit-1). The encoded proteins are putative secreted proteins containing EFG-like motifs and leucine-rich repeats, both are conserved protein-protein interaction domains. Slit-1, -2, and -3 mRNAs are expressed in the brain, spinal cord, and thyroid, respectively (Itoh, A. et al., (1998) Brain Res. Mol. Brain Res. 62:175-186). The Slit family of proteins are indicated to be functional ligands of glypican-i in nervous tissue and suggests that their interactions may be critical in certain stages during central nervous system histogenesis (Liang, Y. et al., (1999) J. Biol. Chem 274:17885-17892).

Neuropeptides and vasomediators (NP/VM) comprise a large family of endogenous signaling molecules. Included in this family are neuropeptides and neuropeptide hormones such as bombesin, neuropeptide Y, neurotensin, neuromedin N, melanocortins, opioids, galanin, somatostatin, tachykinins, urotensin II and related peptides involved in smooth muscle stimulation, vasopressin, vasoactive intestinal peptide, and circulatory system-borne signaling molecules such as angiotensin, complement, calcitonin, endothelins, formyl-methionyl peptides, glucagon, cholecystokinin and gastrin. NP/VMs can transduce signals directly, modulate the activity or release of other neurotransmitters and hormones, and act as catalytic enzymes in cascades. The effects of NP/VMs range from extremely brief to long-lasting. (Reviewed in Martin, C. R. et al. (1985) Endocrine Physiology, Oxford University Press, New York, N.Y., pp.57-62.)

NP/VMs are involved in numerous neurological and cardiovascular disorders. For example, neuropeptide Y is involved in hypertension, congestive heart failure, affective disorders, and appetite regulation. Somatostatin inhibits secretion of growth hormone and prolactin in the anterior pituitary, as well as inhibiting secretion in intestine, pancreatic acinar cells, and pancreaticbeta-cells. A reduction in somatostatin levels has been reported in Alzheimer's disease and Parkinson's disease. Vasopressin acts in the kidney to increase water and sodium absorption, and in higher concentrations stimulates contraction of vascular smooth muscle, platelet activation, and glycogen breakdown in the liver. Vasopressin and its analogues are used clinically to treat diabetes insipidus. Endothelin and angiotensin are involved in hypertension, and drugs, such as captopril, which reduce plasma levels of angiotensin, are used to reduce blood pressure (Watson, S. and S. Arkinstall (1994) The G-protein Linked Receptor Facts Book, Academic Press, San Diego Calif., pp. 194; 252; 284; 55; 111).

Neuropeptides have also been shown to have roles in nociception (pain). Vasoactive intestinal peptide appears to play an important role in chronic neuropathic pain. Nociceptin, an endogenous ligand for for the opioid receptor-like 1 receptor, is thought to have a predominantly anti-nociceptive effect, and has been shown to have analgesic properties in different animal models of tonic or chronic pain (Dickinson, T. and Fleetwood-Walker, S. M. (1998) Trends Pharmacol. Sci. 19:346-348).

Other proteins that contain signal peptides include secreted proteins with enzymatic activity. Such activity includes, for example, oxidoreductase/dehydrogenase activity, transferase activity, hydrolase activity, lyase activity, isomerase activity, or ligase activity. For example, matrix metalloproteinases are secreted hydrolytic enzymes that degrade the extracellular matrix and thus play an important role in tumor metastasis, tissue morphogenesis, and arthritis (Reponen, P. et al. (1995) Dev. Dyn. 202:388-396; Firestein, G. S. (1992) Curr. Opin. Rheumatol. 4:348-354; Ray, J. M. and Stetler-Stevenson, W. G. (1994) Eur. Respir. J. 7:2062-2072; and Mignatti, P. and Rifkin, D. B. (1993) Physiol. Rev. 73:161-195). Additional examples are the acetyl-CoA synthetases which activate acetate for use in lipid synthesis or energy generation (Luong, A. et al. (2000) J. Biol. Chem. 275:26458-264566). The result of acetyl-CoA synthetase activity is the formation of acetyl-CoA from acetate and CoA. Acetyl-CoA sythetases share a region of sequence similarity identified as the AMP-binding domain signature. Acetyl-CoA synthetase has been shown to be associated with hypertension (H. Toh, (1991) Protein Seq. Data Anal. 4:111-117, and Iwai, N. et. al., (1994) Hypertension 23:375-380).

A number of isomerases catalyze steps in protein folding, phototransduction, and various anabolic and catabolic pathways. One class of isomerases is known as peptidyl-prolyl cis-trans isomerases (PPIases). PPIases catalyze the cis to trans isomerization of certain proline imidic bonds in proteins. Two families of PPIases are the FK506 binding proteins (FKBPs), and cyclophilins (CyPs). FKBPs bind the potent immunosuppressants FK506 and rapamycin, thereby inhibiting signaling pathways in T-cells. Specifically, the PPIase activity of FKBPs is inhibited by binding of FK506 or rapamycin. There are five members of the FKBP family which are named according to their calculated molecular masses (FKBP12, FKBP13, FKBP25, FKBP52, and FKBP65), and localized to different regions of the cell where they associate with different protein complexes (Coss, M. et al. (1995) J. Biol. Chem. 270:29336-29341; Schreiber, S. L. (1991) Science 251:283-287).

The peptidyl-prolyl isomerase activity of CyP may be part of the signaling pathway that leads to T-cell activation. CyP isomerase activity is associated with protein folding and protein trafficking, and may also be involved in assembly/disassembly of protein complexes and regulation of protein activity. For example, in Drosophila, the CyP NinaA is required for correct localization of rhodopsins, while a mammalian CyP (Cyp40) is part of the Hsp90/Hsc70 complex that binds steroid receptors. The mammalian CypA has been shown to bind the gag protein from human immunodeficiency virus 1 (HIV-1), an interaction that can be inhibited by cyclosporin. Since cyclosporin has potent anti-HIV-1 activity, CypA may play an essential function in HIV-1 replication. Finally, Cyp40 has been shown to bind and inactivate the transcription factor c-Myb, an effect that is reversed by cyclosporin. This effect implicates CyPs in the regulation of transcription, transformation, and differentiation (Bergsma, D. J. et al (1991) J. Biol. Chem. 266:23204-23214; Hunter, T. (1998) Cell 92: 141-143; and Leverson, J. D. and Ness, S. A. (1998) Mol. Cell. 1:203-211).

Gamma-carboxyglutamic acid (Gla) proteins rich in proline (PRGPs) are members of a family of vitamin K-dependent single-pass integral membrane proteins. These proteins are characterized by an extracellular amino terminal domain of approximately 45 amino acids rich in Gla. The intracellular carboxyl terminal region contains one or two copies of the sequence PPXY, a motif present in a variety of proteins involved in such diverse cellular functions as signal transduction, cell cycle progression, and protein turnover (Kulman, J. D. et al., (2001) Proc. Natl. Acad. Sci. U.S.A. 98:1370-1375). The process of post-translational modification of glutamic residues to form Gla is Vitamin K-dependent carboxylation. Proteins which contain Gla include plasma proteins involved in blood coagulation. These proteins are prothrombin, proteins C, S, and Z, and coagulation factors VII, IX, and X. Osteocalcin (bone-Gla protein, BGP) and matrix Gla-protein (MGP) also contain Gla Friedman, P. A., and C. T. Przysiecki (1987) Int. J. Biochem. 19:1-7; C. Vermeer (1990) Biochem. J. 266:625-636).

The Drosophila sp. gene crossveinless 2 is characterized as having a putative signal or transmembrane sequence, and a partial Von Willebrand Factor D domain similar to those domains known to regulate the formation of intramolecular and intermolecular bonds and five cysteine-rich domains, known to bind BMP-like (bone morphogenetic proteins) ligands. These features suggest that crossveinless 2 may act extracelluarly or in the secretory pathway to directly potentiate ligand signaling and hence, involvement in the BMP-like signaling pathway known to play a role in vein specification (Conley, C. A. et al., (2000) Development 127:3947-3959). The dorsal-ventral pattering in both vertebrate and Drosophila embryos requires a conserved system of extracellular proteins to generate a positional information gradient.

The presence of signal peptides has also been seen in proteins associated with prostate cancer. The development of androgen-independent cells in prostate tumors is useful as a method of determining disease progression. As with most tumors, prostate cancer develops through a multistage progression ultimately resulting in an aggressive tumor phenotype. The initial step in tumor progression involves the hyperproliferation of normal luminal and/or basal epithelial cells. Androgen responsive cells become hyperplastic and evolve into early-stage tumors. Although early-stage tumors are often androgen-sensitive and respond to androgen ablation, a population of androgen-independent cells evolve from the hyperplastic population. These cells represent a more advanced form of prostate tumor that may become invasive and potentially become metastatic to the bone, brain, or lung.

Expression Profiling

Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Foam Cell Formation and Secreted Proteins

Atherosclerosis and the associated coronary artery disease and cerebral stroke represent the most common cause of death in industrialized nations. Although certain key risk factors have been identified, a full molecular characterization that elucidates the causes and provides care for this complex disease has not been achieved. Molecular characterization of growth and regression of atherosclerotic vascular lesions requires identification of the genes that contribute to features of the lesion including growth, stability, dissolution, rupture and, most lethally, induction of occlusive vessel thrombus.

An early step in the development of atherosclerosis is formation of the “fatty streak”. Lipoproteins, such as the cholesterol-rich low-density lipoprotein (LDL), accumulate in the extracellular space of the vascular intima, and undergo modification. Oxidation of LDL occurs most avidly in the sub-endothelial space where circulating antioxidant defenses are less effective. The degree of LDL oxidation affects its interaction with target cells. “Minimly oxidized” LDL (MM-LDL) is able to bind to LDL receptor but not to the oxidized LDL (Ox-LDL) or “scavenger” receptors that have been identified, including scavenger receptor types A and B, CD36, CD68/macrosialin and LOX-1 (Navab et al. (1994) Arterioscler Thromb Vasc Biol 16:831-842; Kodama et al. (1990) Nature 343:531-535; Acton et al. (1994) J Biol Chem 269:21003-21009; Eademann et al. (1993) J Biol Chem 268:11811-11816; Ramprasad et al. (1996) Proc Natl Acad Sci 92:14833-14838; Kataoka et al. (1999) Circulation 99:3110-3117). MM-LDL can increase the adherence and penetration of monocytes, stimulate the release of monocyte chemotactic protein 1 (MCP-1) by endothelial cells, and induce scavenger receptor A (SRA) and CD36 expression in macrophages (Cushing et al. (1990) Proc Natl Acad Sci 87:5134-5138; Yoshida et al. (1998) Arterioscler Thromb Vasc Biol 18:794-802; Steinberg (1997) J Biol Chem 272:20963-20966). SRA and the other scavenger receptors can bind Ox-LDL and enhance uptake of lipoprotein particles.

Mononuclear phagocytes enter the intima, differentiate into macrophages, and ingest modified lipids including Ox-LDL. In most cell types, cholesterol content is tightly controlled by feedback regulation of LDL receptors and biosynthetic enzymes (Brown and Goldstein (1986) Science 232:34-47). In macrophages, however, the additional scavenger receptors lead to unregulated uptake of cholesterol (Brown and Goldstein (1983) Annu Rev Biochem 52:223-261) and accumulation of multiple intracellular lipid droplets producing a “foam cell” phenotype. Cholesterol-engorged and dead macrophages contribute most of the mass of early “fatty streak” plaques and typical “advanced” lesions of diseased arteries. Numerous studies have described a variety of foam cell responses that contribute to growth and rupture of atherosclerotic vessel wall plaques. These responses include production of multiple growth factors and cytokines, which promote proliferation and adherence of neighboring cells; chemokines, which further attract circulating monocytes into the growing plaque; proteins, which cause remodeling of the extracellular matrix; and tissue factor, which can trigger thrombosis (Ross (1993) Nature 362:801-809; Quin et al. (1987) Proc Natl Acad Sci 84:2995-2998). Thus, cholesterol-loaded macrophages which occur in abundance in most stages of the atherosclerotic plaque formation contribute to inception of the atheroscerotic process and to eventual plaque rupture and occlusive thrombus.

During Ox-LDL uptake, macrophages produce cytokines and growth factors that elicit further cellular events that modulate atherogenesis such as smooth muscle cell proliferation and production of extracellular matrix. Additionally, these macrophages may activate genes involved in inflammation including inducible nitric oxide synthase. Thus, genes differentiatly expressed during foam cell formation may reasonably be expected to be markers of the atherosclerotic process.

Steroid Molecules and Secreted Proteins

The potential application of gene expression profiling is particularly relevant to measuring the toxic response to potential therapeutic compounds and of the metabolic response to therapeutic agents. Diseases treated with steroids and disorders caused by the metabolic response to treatment with steroids include adenomatosis, cholestasis, cirrhosis, hemangioma, Henoch-Schonlein purpura, hepatitis, hepatocellular and metastatic carcinomas, idiopathic thrombocytopenic purpura, porphyria, sarcoidosis, and Wilson disease. Response may be measured by comparing both the levels and sequences expressed in tissues from subjects exposed to or treated with steroid compounds such as mifepristone, progesterone, beclomethasone, medroxyprogesterone, budesonide, prednisone, dexamethasone, betamethasone, or danazol with the levels and sequences expressed in normal untreated tissue. Steroids are a class of lipid-soluble molecules, including cholesterol, bile acids, vitamin D, and hormones, that share a common four-ring structure based on cyclopentanoperhydrophenantbrene and that carrry out a wide variety of functions. Cholesterol, for example, is a component of cell membranes that controls membrane fluidity. It is also a precursor for bile acids which solubilize lipids and facilitate absorption in the small intestine during digestion. Vitamin D regulates the absorption of calcium in the small intestine and controls the concentration of calcium in plasma. Steroid hormones, produced by the adrenal cortex, ovaries, and testes, include glucocorticoids, mineralocorticoids, androgens, and estrogens. They control various biological processes by binding to intracellular receptors that regulate transcription of specific genes in the nucleus. Glucocorticoids, for example, increase blood glucose concentrations by regulation of gluconeogenesis in the liver, increase blood concentrations of fatty acids by promoting lipolysis in adipose tissues, modulate sensitivity to catcholamines in the central nervous system, and reduce inflammation. The principal mineralocorticoid, aldosterone, is produced by the adrenal cortex and acts on cells of the distal tubules of the kidney to enhance sodium ion reabsorption. Androgens, produced by the interstitial cells of Leydig in the testis, include the male sex hormone testosterone, which triggers changes at puberty, the production of sperm and maintenance of secondary sexual characteristics. Female sex hormones, estrogen and progesterone, are produced by the ovaries and also by the placenta and adrenal cortex of the fetus during pregnancy. Estrogen regulates female reproductive processes and secondary sexual characteristics. Progesterone regulates changes in the endometrium during the menstrual cycle and pregnancy.

Steroid hormones are widely used for fertility control and in anti-inflammatory treatments for physical injuries and diseases such as arthritis, asthma, and auto-immune disorders. Progesterone, a naturally occurring progestin is primarily used to treat amenorrhea, abnormal uterine bleeding, or as a contraceptive. Endogenous progesterone is responsible for inducing secretory activity in the endometrium of the estrogen-primed uterus in preparation for the implantation of a fertilized egg and for the maintenance of pregnancy. It is secreted from the corpus luteum in response to luteinizing hormone (LH). The primary contraceptive effect of exogenous progestins involves the suppression of the midcycle surge of LH. At the cellular level, progestins diffuse freely into target cells and bind to the progesterone receptor. Target cells include the female reproductive tract, the mammary gland, the hypothalamus, and the pituitary. Once bound to the receptor, progestins slow the frequency of release of gonadotropin releasing hormone from the hypothalamus and blunt the pre-ovulatory LH surge, thereby preventing foilicular maturation and ovulation. Progesterone has minimal estrogenic and androgenic activity. Progesterone is metabolized hepatically to pregnanediol and conjugated with glucuronic acid.

Medroxyprogesterone (MAM), also known as 6α-methyl-17-hydroxyprogesterone, is a synthetic progestin with a pharmacological activity about 15 times greater than progesterone. MAH is used for the treatment of renal and endometrial carcinomas, amenorrhea, abnormal uterine bleeding, and endometriosis associated with hormonal imbalance. MAH has a stimulatory effect on respiratory centers and has been used in cases of low blood oxygenation caused by sleep apnea, chronic obstructive pulmonary disease, or hypercapnia.

Mifepristone, also known as RU-486, is an antiprogesterone drug that blocks receptors of progesterone. It counteracts the effects of progesterone, which is needed to sustain pregnancy. Mifepristone induces spontaneous abortion when administered in early pregnancy followed by treatment with the prostaglandin, misoprostol. Further, studies show that mifepristone at a substantially lower dose can be highly effective as a postcoital contraceptive when administered within five days after unprotected intercourse, thus providing women with a “morning-after pill” in case of contraceptive failure or sexual assault. Mifepristone also has potential uses in the treatment of breast and ovarian cancers in cases in which tumors are progesterone-dependent. It interferes with steroid-dependent growth of brain meningiomas, and may be useful in treatment of endometriosis where it blocks the estrogen-dependent growth of endometrial tissues. It may also be useful in treatment of uterine fibroid tumors and Cushing's Syndrome. Mifepristone binds to glucocorticoid receptors and interferes with cortisol binding. Mifepristone also may act as an anti-glucocorticoid and be effective for treating conditions where cortisol levels are elevated such as AIDS, anorexia nervosa, ulcers, diabetes, Parkinson's disease, multiple sclerosis, and Alzheimer's disease.

Danazol is a synthetic steroid derived from ethinyl testosterone. Danazol indirectly reduces estrogen production by lowering pituitary synthesis of follicle-stimulating hormone and LH. Danazol also binds to sex hormone receptors in target tissues, thereby exhibiting anabolic, antiestrognic, and weakly androgenic activity. Danazol does not possess any progestogenic activity, and does not suppress normal pituitary release of corticotropin or release of cortisol by the adrenal glands. Danazol is used in the treatment of endometriosis to relieve pain and inhibit endometrial cell growth. It is also used to treat fibrocystic breast disease and hereditary angioedema.

Corticosteroids are used to relieve inflammation and to suppress the immune response. They inhibit eosinophil, basophil, and airway epithelial cell function by regulation of cytokines that mediate the inflammatory response. They inhibit leukocyte infiltration at the site of inflammation, interfere in the function of mediators of the inflammatory response, and suppress the humoral immune response. Corticosteroids are used to treat allergies, asthma, arthritis, and skin conditions. Beclomethasone is a synthetic glucocorticoid that is used to treat steroid-dependent asthma, to relieve symptoms associated with allergic or nonallergic (vasomotor) rhinitis, or to prevent recurrent nasal polyps following surgical removal. The anti-inflammatory and vasoconstrictive effects of intranasal beclomethasone are 5000 times greater than those produced by hydrocortisone. Budesonide is a corticosteroid used to control symptoms associated with allergic rhinitis or asthma. Budesonide has high topical anti-inflammatory activity but low systemic activity. Dexamethasone is a synthetic glucocorticoid used in anti-inflammatory or immunosuppressive compositions. It is also used in inhalants to prevent symptoms of asthma. Due to its greater ability to reach the central nervous system, dexamethasone is usually the treatment of choice to control cerebral edema. Dexamethasone is approximately 20-30 times more potent than hydrocortisone and 5-7 times more potent than prednisone. Prednisone is metabolized in the liver to its active form, prednisolone, a glucocorticoid with anti-inflammatory properties. Prednisone is approximately 4 times more potent than hydrocortisone and the duration of action of prednisone is intermediate between hydrocortisone and dexamethasone. Prednisone is used to treat allograft rejection, asthma, systemic lupus erythematosus, arthritis, ulcerative colitis, and other inflammatory conditions. Betamethasone is a synthetic glucocorticoid with antiinflammatory and immunosuppressive activity and is used to treat psoriasis and fungal infections, such as athlete's foot and ringworm.

The anti-inflammatory actions of corticosteroids are thought to involve phospholipase A₂ inhibitory proteins, collectively called lipocortins. Lipocortins, in turn, control the biosynthesis of potent mediators of inflammation such as prostaglandins and leukotrienes by inhibiting the release of the precursor molecule arachidonic acid. Proposed mechanisms of action include decreased IgE synthesis, increased number of β-adrenergic receptors on leukocytes, and decreased arachidonic acid metabolism. During an immediate allergic reaction, such as in chronic bronchial asthma, allergens bridge the IgE antibodies on the surface of mast cells, which triggers these cells to release chemotactic substances. Mast cell influx and activation, therefore, is partially responsible for the inflammation and hyperirritability of the oral mucosa in asthmatic patients. This inflammation can be retarded by administration of corticosteroids.

The effects upon liver metabolism and hormone clearance mechanisms are important to understand the pharmacodynamics of a drug. The human C3A cell line is a clonal derivative of HepG2/C3 (hepatoma cell line, isolated from a 15-year-old male with liver tumor), which was selected for strong contact inhibition of growth. The use of a clonal population enhances the reproducibility of the cells. C3A cells have many characteristics of primary human hepatocytes in culture: i) expression of insulin receptor and insulin-like growth factor II receptor; ii) secretion of a high ratio of serum albumin compared with α-fetoprotein iii) conversion of ammonia to urea and glutamine; iv) metabolize aromatic amino acids; and v) proliferate in glucose-free and insulin-free medium The C3A cell line is now well established as an in vitro model of the mature human liver (Mickelson et al. (1995) Hepatology 22:866-875; Nagendra et al. (1997) Am J Physiol 272:G408-G416).

The discovery of new secreted proteins, and the polynucleotides encoding them, satisfies a need in the art by providing new compositions which are useful in the diagnosis, prevention, and treatment of liver, cell proliferative, autoimmune/inflammatory, cardiovascular, neurological, and developmental disorders, and in the assessment of the effects of exogenous compounds on the expression of nucleic acid and amino acid sequences of secreted proteins.

### SUMMARY OF THE INVENTION

The invention features purified polypeptides, secreted proteins, referred to collectively as “SECP” and individually as “SECP-1,” “SECP-2,” “SECP-3,” “SECP-4,” “SECP-5,” “SECP-6,” “SECP-7,” “SECP-8,” “SECP-9,” “SECP-10,” “SECP-11,” “SECP-12,” “SECP-13,” “SECP-14,” “SECP-15,” “SECP-16,” “SECP-17,” “SECP-18,” “SECP-19,” “SECP-20,” “SECP-21,” “SECP-22,” “SECP-23,” “SECP-24,” “SECP-25,” “SECP-26,” “SECP-27,” “SECP-28,” “SECP-29,” and “SECP-30.” In one aspect, the invention provides an isolated polypeptide selected from the group consisting of a) a polypeptide comprising an amino acid sequence selected from the group consisting of SEQ ID NO:1-30, b) a polypeptide comprising a naturally occurring amino acid sequence at least 90% identical to an amino acid sequence selected from the group consisting of SEQ ID NO:1-30, c) a biologically active fragment of a polypeptide having an amino acid sequence selected from the group consisting of SEQ ID NO:1-30, and d) an immunogenic fragment of a polypeptide having an amino acid sequence selected from the group consisting of SEQ ID NO:1-30. In one alternative, the invention provides an isolated polypeptide comprising the amino acid sequence of SEQ ID NO:1-30.

The invention farther provides an isolated polynucleotide encoding a polypeptide selected from the group consisting of a) a polypeptide comprising an amino acid sequence selected from the group consisting of SEQ ID NO:1-30, b) a polypeptide comprising a naturally occurring amino acid sequence at least 90% identical to an amino acid sequence selected from the group consisting of SEQ ID NO:1-30, c) a biologically active fragment of a polypeptide having an amino acid sequence selected from the group consisting of SEQ ID NO:1-30, and d) an immunogenic fragment of a polypeptide having an amino acid sequence selected from the group consisting of SEQ ID NO:1-30. In one alternative, the polynucleotide encodes a polypeptide selected from the group consisting of SEQ ID NO:1-30. In another alternative, the polynucleotide is selected from the group consisting of SEQ ID NO:31-60.

Additionally, the invention provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-30, b) a polypeptide comprising a naturally occurring amino acid sequence at least 90% identical to an amino acid sequence selected from the group consisting of SEQ ID NO:1-30, c) a biologically active fragment of a polypeptide having an amino acid sequence selected from the group consisting of SEQ ID NO:1-30, and d) an immunogenic fragment of a polypeptide having an amino acid sequence selected from the group consisting of SEQ ID NO:1-30. In one alternative, the invention provides a cell transformed with the recombinant polynucleotide. In another alternative, the invention provides a transgenic organism comprising the recombinant polynucleotide.

The invention also provides a method for producing a polypeptide selected from the group consisting of a) a polypeptide comprising an amino acid sequence selected from the group consisting of SEQ ID NO:1-30, b) a polypeptide comprising a naturally occurring amino acid sequence at least 90% identical to an amino acid sequence selected from the group consisting of SEQ ID NO:1-30, c) a biologically active fragment of a polypeptide having an amino acid sequence selected from the group consisting of SEQ ID NO:1-30, and d) an immunogenic fragment of a polypeptide having an amino acid sequence selected from the group consisting of SEQ ID NO:1-30. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Additionally, the invention provides an isolated antibody which specifically binds to a polypeptide selected from the group consisting of a) a polypeptide comprising an amino acid sequence selected from the group consisting of SEQ ID NO:1-30, b) a polypeptide comprising a naturally occurring amino acid sequence at least 90% identical to an amino acid sequence selected from the group consisting of SEQ ID NO:1-30, c) a biologically active fragment of a polypeptide having an amino acid sequence selected from the group consisting of SEQ ID NO:1-30, and d) an immunogenic fragment of a polypeptide having an amino acid sequence selected from the group consisting of SEQ ID NO:1-30.

The invention further provides an isolated polynucleotide selected from the group consisting of a) a polynucleotide comprising a polynucleotide sequence selected from the group consisting of SEQ ID NO:31-60, b) a polynucleotide comprising a naturally occurring polynucleotide sequence at least 90% identical to a polynucleotide sequence selected from the group consisting of SEQ ID NO:31-60, c) a polynucleotide complementary to the polynucleotide of a), d) a polynucleotide complementary to the polynucleotide of b), and e) an RNA equivalent of a)-d). In one alternative, the polynucleotide comprises at least 60 contiguous nucleotides.

Additionally, the invention provides a method for detecting a target polynucleotide in a sample, said target polynucleotide having a sequence of a polynucleotide selected from the group consisting of a) a polynucleotide comprising a polynucleotide sequence selected from the group consisting of SEQ ID NO:31-60, b) a polynucleotide comprising a naturally occurring polynucleotide sequence at least 90% identical to a polynucleotide sequence selected from the group consisting of SEQ ID NO:31-60, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex, and optionally, if present, the amount thereof. In one alternative, the probe comprises at least 60 contiguous nucleotides.

The invention further provides a method for detecting a target polynucleotide in a sample, said target polynucleotide having a sequence of a polynucleotide selected from the group consisting of a) a polynucleotide comprising a polynucleotide sequence selected from the group consisting of SEQ ID NO:31-60, b) a polynucleotide comprising a naturally occurring polynucleotide sequence at least 90% identical to a polynucleotide sequence selected from the group consisting of SEQ ID NO:31-60, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof, and, optionally, if present, the amount thereof.

The invention further provides a composition comprising an effective amount of a polypeptide selected from the group consisting of a) a polypeptide comprising an amino acid sequence selected from the group consisting of SEQ ID NO:1-30, b) a polypeptide comprising a naturally occurring amino acid sequence at least 90% identical to an amino acid sequence selected from the group consisting of SEQ ID NO:1-30, c) a biologically active fragment of a polypeptide having an amino acid sequence selected from the group consisting of SEQ ID NO:1-30, and d) an immunogenic fragment of a polypeptide having an amino acid sequence selected from the group consisting of SEQ ID NO:1-30, and a pharmaceutically acceptable excipient. In one embodiment, the composition comprises an amino acid sequence selected from the group consisting of SEQ ID NO:1-30. The invention additionally provides a method of treating a disease or condition associated with decreased expression of functional SECP, comprising administering to a patient in need of such treatment the composition.

The invention also provides a method for screening a compound for effectiveness as an agonist of a polypeptide selected from the group consisting of a) a polypeptide comprising an amino acid sequence selected from the group consisting of SEQ ID NO:1-30, b) a polypeptide comprising a naturally occurring amino acid sequence at least 90% identical to an amino acid sequence selected from the group consisting of SEQ ID NO:1-30, c) a biologically active fragment of a polypeptide having an amino acid sequence selected from the group consisting of SEQ ID NO:1-30, and d) an immunogenic fragment of a polypeptide having an amino acid sequence selected from the group consisting of SEQ ID NO:1-30. The method comprises a) exposing a sample comprising the polypeptide to a compound, and b) detecting agonist activity in the sample. In one alternative, the invention provides a composition comprising an agonist compound identified by the method and a pharmaceutically acceptable excipient. In another alternative, the invention provides a method of treating a disease or condition associated with decreased expression of functional SECP, comprising administering to a patient in need of such treatment the composition.

Additionally, the invention provides a method for screening a compound for effectiveness as an antagonist of a polypeptide selected from the group consisting of a) a polypeptide comprising an amino acid sequence selected from the group consisting of SEQ ID NO:1-30,b) a polypeptide comprising a naturally occurring amino acid sequence at least 90% identical to an amino acid sequence selected from the group consisting of SEQ ID NO:1-30, c) a biologically active fragment of a polypeptide having an amino acid sequence selected from the group consisting of SEQ ID NO:1-30, and d) an immunogenic fragment of a polypeptide having an amino acid sequence selected from the group consisting of SEQ ID NO:1-30. The method comprises a) exposing a sample comprising the polypeptide to a compound, and b) detecting antagonist activity in the sample. In one alternative, the invention provides a composition comprising an antagonist compound identified by the method and a pharmaceutically acceptable excipient. In another alternative, the invention provides a method of treating a disease or condition associated with overexpression of functional SECP, comprising administering to a patient in need of such treatment the composition.

The invention further provides a method of screening for a compound that specifically binds to a polypeptide selected from the group consisting of a) a polypeptide comprising an amino acid sequence selected from the group consisting of SEQ ID NO:1-30, b) a polypeptide comprising a naturally occurring amino acid sequence at least 90% identical to an amino acid sequence selected from the group consisting of SEQ ID NO:1-30, c) a biologically active fragment of a polypeptide having an amino acid sequence selected from the group consisting of SEQ ID NO:1-30, and d) an immunogenic fragment of a polypeptide having an amino acid sequence selected from the group consisting of SEQ ID NO:1-30. The method comprises a) combining the polypeptide with at least one test compound under suitable conditions, and b) detecting binding of the polypeptide to the test compound, thereby identifying a compound that specifically binds to the polypeptide.

The invention further provides a method of screening for a compound that modulates the activity of a polypeptide selected from the group consisting of a) a polypeptide comprising an amino acid sequence selected from the group consisting of SEQ ID NO:1-30, b) a polypeptide comprising a naturally occurring amino acid sequence at least 90% identical to an amino acid sequence selected from the group consisting of SEQ ID NO:1-30, c) a biologically active fragment of a polypeptide having an amino acid sequence selected from the group consisting of SEQ ID NO:1-30, and d) an immunogenic fragment of a polypeptide having an amino acid sequence selected from the group consisting of SEQ ID NO:1-30. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

The invention further provides a method for screening a compound for effectiveness in altering expression of a target polynucleotide, wherein said target polynucleotide comprises a polynucleotide sequence selected from the group consisting of SEQ ID NO:31-60, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

The invention further provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:31-60, ii) a polynucleotide comprising a naturally occurring polynucleotide sequence at least 90% identical to a polynucleotide sequence selected from the group consisting of SEQ ID NO:31-60, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ D NO:31-60, ii) a polynucleotide comprising a naturally occurring polynucleotide sequence at least 90% identical to a polynucleotide sequence selected from the group consisting of SEQ ID NO:31-60, iii) a polynucleotide complementary to the polynucleotide of i), iv) a polynucleotide complementary to the polynucleotide of ii), and v) an RNA equivalent of i)-iv). Alternatively, the target polynucleotide comprises a fragment of a polynucleotide sequence selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for the full length polynucleotide and polypeptide sequences of the present invention.

Table 2 shows the GenBank identification number and annotation of the nearest GenBank homolog, and the PROTEOME database identification numbers and annotations of PROTEOME database homologs, for polypeptides of the invention The probability scores for the matches between each polypeptide and its homolog(s) are also shown.

Table 3 shows structural features of polypeptide sequences of the invention, including predicted motifs and domains, along with the methods, algorithms, and searchable databases used for analysis of the polypeptides.

Table 4 lists the cDNA and/or genomic DNA fragments which were used to assemble polynucleotide sequences of the invention, along with selected fragments of the polynucleotide sequences.

Table 5 shows the representative cDNA library for polynucleotides of the invention.

Table 6 provides an appendix which describes the tissues and vectors used for construction of the cDNA libraries shown in Table 5.

Table 7 shows the tools, programs, and algorithms used to analyze the polynucleotides and polypeptides of the invention, along with applicable descriptions, references, and threshold parameters.

Table 8 shows single nucleotide polymorphisms found in polynucleotide sequences of the invention, along with allele frequencies in different human populations.

### DESCRIPTION OF THE INVENTION

Before the present proteins, nucleotide sequences, and methods are described, it is understood that this invention is not limited to the particular machines, materials and methods described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“SECP” refers to the amino acid sequences of substantially purified SECP obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of SECP. Agonists may include proteins, nucleic acids, carbohydrates, small molecules, or any other compound or composition which modulates the activity of SECP either by directly interacting with SECP or by acting on components of the biological pathway in which SECP participates.

An “allelic variant” is an alternative form of the gene encoding SECP. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding SECP include those sequences with deletions, insertions, or substitutions of different nucleotides, resulting in a polypeptide the same as SECP or a polypeptide with at least one functional characteristic of SECP. Included within this definition are polymorphisms which may or may not be readily detectable using a particular oligonucleotide probe of the polynucleotide encoding SECP, and improper or unexpected hybridization to allelic variants, with a locus other than the normal chromosomal locus for the polynucleotide sequence encoding SECP. The encoded protein may also be “altered,” and may contain deletions, insertions, or substitutions of amino acid residues which produce a silent change and result in a functionally equivalent SECP. Deliberate amino acid substitutions may be made on the basis of similarity in polarity, charge, solubility, hydrophobicity, hydrophilicity, and/or the amphipathic nature of the residues, as long as the biological or immunological activity of SECP is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” refer to an oligopeptide, peptide, polypeptide, or protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid sequence. Amplification is generally carried out using polymerase chain reaction (PCR) technologies well known in the art.

The term “antagonist” refers to a molecule which inhibits or attenuates the biological activity of SECP. Antagonists may include proteins such as antibodies, nucleic acids, carbohydrates, small molecules, or any other compound or composition which modulates the activity of SECP either by directly interacting with SECP or by acting on components of the biological pathway in which SECP participates.

The term “antibody” refers to intact immunoglobulin molecules as well as to fragments thereof, such as Fab, F(ab′)₂, and Fv fragments, which are capable of binding an epitopic determinant. Antibodies that bind SECP polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The tern “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (articular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker. (See, e.g., Brody, E. N. and L. Gold (2000) J. Biotechnol. 74:5-13.)

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic SECP, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide sequence” and a “composition comprising a given amino acid sequence” refer broadly to any composition containing the given polynucleotide or amino acid sequence. The composition may comprise a dry formulation or an aqueous solution. Compositions comprising polynucleotide sequences encoding SECP or fragments of SECP may be employed as hybridization probes. The probes may be stored in freeze-dried form and may be associated with a stabilzing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalendy or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of SECP or the polynucleotide encoding SECP which is identical in sequence to but shorter in length than the parent sequence. A fragment may comprise up to the entire length of the defined sequence, minus one nucleotide/amino acid residue. For example, a fragment may comprise from 5 to 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:31-60 comprises a region of unique polynucleotide sequence that specifically identifies SEQ ID NO:31-60, for example, as distinct from any other sequence in the genome from which the fragment was obtained. A fragment of SEQ ID NO:31-60 is useful, for example, in hybridization and amplification technologies and in analogous methods that distinguish SEQ ID NO:31-60 from related polynucleotide sequences. The precise length of a fragment of SEQ ID NO:31-60 and the region of SEQ ID NO:31-60 to which the fragment corresponds are routinely determinable by one of ordinary skill in the art based on the intended purpose for the fragment.

A fragment of SEQ ID NO:1-30 is encoded by a fragment of SEQ ID NO:31-60. A fragment of SEQ ID NO:1-30 comprises a region of unique amino acid sequence that specifically identifies SEQ ID NO:1-30. For example, a fragment of SEQ ID NO:1-30 is useful as an immunogenic peptide for the development of antibodies that specifically recognize SEQ ID NO:1-30. The precise length of a fragment of SEQ ID NO:1-30 and the region of SEQ ID NO:1-30 to which the fragment corresponds are routinely determinable by one of ordinary skill in the art based on the intended purpose for the fragment.

A “full length” polynucleotide sequence is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorithim Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polynucleotide sequences.

Alternatively, a suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gov/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (April-21-2000) set at default parameters. Such default parameters may be, for example:

Matrix: BLOSUM62

Reward for match: 1

Penalty for mismatch: 2

Open Gap: 5 and Extension Gap: 2 penalties

Gap x drop-off: 50

Expect: 10

Word Size: 11

Filter: on

Percent identity may be measured over the length of an entire defined sequence, for example, as defined by a particular SEQ D number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr.-21-2000) with blastp set at default parameters. Such default parameters may be, for example:

Matrix:.BLOSUM62

Open Gap: 11 and Extension Gap: 1 penalties

Gap x drop-off: 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68 ° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acid sequences by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The words “insertion” and “addition” refer to changes in an amino acid or nucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of SECP which is capable of eliciting an immune response when introduced into a living organism, for example, a mammal. The term “immunogenic fragment” also includes any polypeptide or oligopeptide fragment of SECP which is useful in any of the antibody production methods disclosed herein or known in the art.

The term “microarray” refers to an arrangement of a plurality of polynucleotides, polypeptides, or other chemical compounds on a substrate.

The terms “element” and “array element” refer to a polynucleotide, polypeptide, or other chemical compound having a unique and defined position on a microarray.

The term “modulate” refers to a change in the activity of SECP. For example, modulation may cause an increase or a decrease in protein activity, binding characteristics, or any other biological, functional, or immunological properties of SECP.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an SECP may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of SECP.

“Probe” refers to nucleic acid sequences encoding SECP, their complements, or fragments thereof, which are used to detect identical, allelic or related nucleic acid sequences. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid sequence, e.g., by the polymerase chain reaction (PCR).

Probes and primers as used in the present invention typically comprise at least 15 contiguous nucleotides of known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook, J. et al. (1989) Molecular Cloning: A Laboratory Manual, 2^{nd} ed., vol. 1-3, Cold Spring Harbor Press, Plainview N.Y.; Ausubel, F. M. et al. (1987) Current Protocols in Molecular Biology, Greene Publ. Assoc. & Wiley-Intersciences, New York N.Y.; Innis, M. et al. (1990) PCR Protocols, A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify filly or partially complementary polynucleotides in a sample of nucleic acids. Methods of oligonucleotide selection are not limited to those described above.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose.

The term “sample” is used in its broadest sense. A sample suspected of containing SECP, nucleic acids encoding SECP, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least 60% free, preferably at least 75% free, and most preferably at least 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In one alternative, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May-07-1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 55%; at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional fiuctional domains or lack domains that are present in the reference molecule. Species variants are polynucleotide sequences that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May-07-1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

### THE INVENTION

The invention is based on the discovery of new human secreted proteins (SECP), the polynucleotides encoding SECP, and the use of these compositions for the diagnosis, treatment, or prevention of liver, cell proliferative, autoimmune/inflammatory, cardiovascular, neurological, and developmental disorders.

Table 1 summarizes the nomenclature for the full length polynucleotide and polypeptide sequences of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to the polypeptide and polynucleotide sequences of the invention. The full length clones encode polypeptides which have at least 95% sequence identity to the polypeptide sequences shown in column 3.

Table 2 shows sequences with homology to the polypeptides of the invention as identified by BLAST analysis against the GenBank protein (genpept) database and the PROTEOME database. Columns 1 and 2 show the polypeptidese identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention Column 3 shows the number of amino acid residues in each polypeptide. Column 4 shows potential phosphorylation sites, and column 5 shows potential glycosylation sites, as determined by the MOTIFS program of the GCG sequence analysis software package (Genetics Computer Group, Madison Wis.). Column 6 shows amino acid residues comprising signature sequences, domains, and motifs including the locations of signal peptides (as indicated by “Signal Peptide” and/or “signal_cleavage”). Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are secreted proteins.

For example, SEQ ID NO:1 is 99% identical, from residue G18 to residue T289, to human IgG Fc receptor I (GenBank ID g180279) as determined by the Basic Local Alignment Search Tool (BLAST). (See Table 2.) The BLAST probability score is 5.4e-146, which indicates the probability of obtaining the observed polypeptide sequence alignment by chance. SEQ ID NO:1 also contains immunoglobulin domains as determined by searching for statistically significant matches in the hidden Markov model (HMM)-based PFAM database of conserved protein family domains. (See Table 3.) Data from BLIMPS analyses provide further corroborative evidence that SEQ ID NO:1 is a secreted protein (note that “immunoglobulin domains” are characteristic of matrix proteins).

As another example, SEQ ID NO:11 is 150 residues in length and is 100% identical, from residue M1 to residue S105, to human transmembrane gamma-carboxyglutamic acid protein 4 (TGM4) (GenBank ID g12656635) as determined by the Basic Local Alignment Search Tool (BIAST). (See Table 2.) The BLAST probability score is 8.4e-54, which indicates the probability of obtaining the observed polypeptide sequence alignment by chance. SEQ ID NO:11 also contains a Vitamin K-dependent carboxylation/gamma-carboxyglutamic (GLA) domain as determined by searching for statistically significant matches in the hidden Markov model (HMM)-based PFAM database of conserved protein family domains. (See Table 3.) Data from BLIMPS, MOTIFS, and PROFILESCAN analyses provide further corroborative evidence that SEQ ID NO:11 is a member of a family of vitamin K-dependent single-pass integral membrane proteins.

As another example, SEQ ID NO:16 is 519 amino acids in length and is 100% identical, from residue M274 to residue E519 to human AD021 protein (GenBank ID g7578787) as determined by the Basic Local Alignment Search Tool (BLAST). (See Table 2.) The BLAST probability score is 8.3e-134, which indicates the probability of obtaining the observed polypeptide sequence alignment by chance.

As another example, SEQ ID NO:22 is 81% identical, from residue M1 to residue Q138,to murine putative protein sequence (GenBank ID g12845943) as determined by the Basic Local Alignment Search Tool (BLAST). (See Table 2.) The BLAST probability score is 9.1e-57, which indicates the probability of obtaining the observed polypeptide sequence alignment by chance. SEQ ID NO:22 also contains signal cleavage sites as determined by searching for statistically significant matches in the SPScan weight matrix analysis program (See Table 3.)

As another example, SEQ ID NO:23 is 28% identical, fromresidue S42 to residue P345, to human Mac-2 binding protein (GenBank ID g307153) as determined by the Basic Local Alignment Search Tool (BLAST). (See Table 2.) The BLAST probability score is 9.3e-27, which indicates the probability of obtaining the observed polypeptide sequence alignment by chance. SEQ ID NO:23 also contains a BTB/POZ domain as determined by searching for statistically significant matches in the hidden Markov model (HMM)-based PFAM database of conserved protein family domains. (See Table 3.)

SEQ ID NO:2-10, SEQ ID NO:12-15, SEQ ID NO:17-21, and SEQ ID NO:24-30 were analyzed and annotated in a similar manner. The algorithms and parameters for the analysis of SEQ ID NO:1-30 are described in Table 7.

As shown in Table 4, the full length polynucleotide sequences of the present invention were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide sequences of the invention, and of fragments of the polynucleotide sequences which are useful, for example, in hybridization or amplification technologies that identify SEQ ID NO:31-60 or that distinguish between SEQ ID NO:31-60 and related polynucleotide sequences.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotide sequences. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (i.e., those sequences including the designation “NP”). Alternatively, the polynucleotide fragments described in column 2 may refer to assemblages of both cDNA and Genscan-predicted exons brought together by an “exon stitching” algorithm For example, a polynucleotide sequence identified as FL_XXXXXXX_N₁₋N₂₋YYYYY_N₃₋N₄ represents a “stitched” sequence in which XXXXXX is the identification number of the cluster of sequences to which the algorithm was applied, and YYYYY is the number of the prediction generated by the algorithm, and N_{1,2,3, . . . ,} if present, represent specific exons that may have been manually edited during analysis (See Example V). Alternatively, the polynucleotide fragments in column 2 may refer to assemblages of exons brought together by an “exon-stretching” algorithm For example, a polynucleotide sequence identified as FLXXXXXX_gAAAAA_gBBBBB_{—}1_N is a “stretched” sequence, with XXXXXX being the Incyte project identification number, gAAAAA being the GenBank identification number of the human genomic sequence to which the “exon-stretching” algorithm was applied, GBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, | Exon prediction from genomic sequences using, for |
| GFG, | example, GENSCAN (Stanford University, CA, USA) |
| ENST | or FGENES (Computer Genomics Group, The Sanger |
| | Centre, Cambridge, UK). |
| GBI | Hand-edited analysis of genomic sequences. |
| FL | Stitched or stretched genomic sequences (see |
| | Example V). |
| INCY | Full length transcript and exon prediction from |
| | mapping of EST sequences to the genome. Genomic |
| | location and EST composition data are combined |
| | to predict the exons and resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotide sequences which were assembled using Incyte cDNA sequences. The representative cDNA library is the Incyte cDNA library which is most frequently represented by the Incyte cDNA sequences which were used to assemble and confirm the above polynucleotide sequences. The tissues and vectors which were used to construct the cDNA libraries shown in Table 5 are described in Table 6.

Table 8 shows single nucleotide polymorphisms (SNPs) found in polynucleotide sequences of the invention, along with allele frequencies in different human populations. Columns 1 and 2 show the polynucleotide sequence identification number (SEQ ID NO:) and the corresponding Incyte project identification number (PID) for polynucleotides of the invention. Column 3 shows the Incyte identification number for the EST in which the SNP was detected (EST ID), and column 4 shows the identification number for the SNP (SNP ID). Column 5 shows the position within the EST sequence at which the SNP is located (EST SNP), and column 6 shows the position of the SNP within the full-length polynucleotide sequence (CB1 SNP). Column 7 shows the allele found in the EST sequence. Columns 8 and 9 show the two alleles found at the SNP site. Column 10 shows the amino acid encoded by the codon including the SNP site, based upon the allele found in the EST. Columns 11-14 show the frequency of allele 1 in four different human populations. An entry of n/d (not detected) indicates that the frequency of allele 1 in the population was too low to be detected, while n/a (not available) indicates that the allele frequency was not determined for the population.

The invention also encompasses SECP variants. A preferred SECP variant is one which has at least about 80%, or alternatively at least about 90%, or even at least about 95% amino acid sequence identity to the SECP amino acid sequence, and which contains at least one functional or structural characteristic of SECP.

The invention also encompasses polynucleotides which encode SECP. In a particular embodiment, the invention encompasses a polynucleotide sequence comprising a sequence selected from the group consisting of SEQ ID NO:31-60, which encodes SECP. The polynucleotide sequences of SEQ ID NO:31-60, as presented in the Sequence Listing, embrace the equivalent RNA sequences, wherein occurrences of the nitrogenous base thymine are replaced with uracil, and the sugar backbone is composed of ribose instead of deoxynbose.

The invention also encompasses a variant of a polynucleotide sequence encoding SECP. In particular, such a variant polynucleotide sequence will have at least about 70%, or alternatively at least about 85%, or even at least about 95% polynucleotide sequence identity to the polynucleotide sequence encoding SECP. A particular aspect of the invention encompasses a variant of a polynucleotide sequence comprising a sequence selected from the group consisting of SEQ ID NO:31-60 which has at least about 70%, or alternatively at least about 85%, or even at least about 95% polynucleotide sequence identity to a nucleic acid sequence selected from the group consisting of SEQ ID NO:31-60. Any one of the polynucleotide variants described above can encode an amino acid sequence which contains at least one functional or structural characteristic of SECP.

In addition, or in the alternative, a polynucleotide variant of the invention is a splice variant of a polynucleotide sequence encoding SECP. A splice variant may have portions which have significant sequence identity to the polynucleotide sequence encoding SECP, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to the polynucleotide sequence encoding SECP over its entire length; however, portions of the splice variant will have at least about 70%, or alternatively at least about 85%, or alternatively at least about 95%, or alternatively 100% polynucleotide sequence identity to portions of the polynucleotide sequence encoding SECP. Any one of the splice variants described above can encode an amino acid sequence which contains at least one functional or structural characteristic of SECP.

It will be appreciated by those skilled in the art that as a result of the degeneracy of the genetic code, a multitude of polynucleotide sequences encoding SECP, some bearing minimal similarity to the polynucleotide sequences of any known and naturally occurring gene, maybe produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring SECP, and all such variations are to be considered as being specifically disclosed.

Although nucleotide sequences which encode SECP and its variants are generally capable of hybridizing to the nucleotide sequence of the naturally occurring SECP under appropriately selected conditions of stringency, it maybe advantageous to produce nucleotide sequences encoding SECP or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding SECP and its derivatives without altering the encoded amino acid sequences include the production of RNA transcripts having more desirable properties, such as a greater half-life, than transcripts produced from the naturally occurring sequence.

The invention also encompasses production of DNA sequences which encode SECP and SECP derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding SECP or any fragment thereof.

Also encompassed by the invention are polynucleotide sequences that are capable of hybridizing to the claimed polynucleotide sequences, and, in particular, to those shown in SEQ ID NO:31-60 and fragments thereof under various conditions of stringency. (See, e.g., Wahl, G. M. and S. L. Berger (1987) Methods Enzymol. 152:399-407; Kimmel, A. R. (1987) Methods Enzymol. 152:507-511.) Hybridization conditions, including annealing and wash conditions, are described in “Definitions.”

Methods for DNA sequencing are well known in the art and may be used to practice any of the embodiments of the invention. The methods may employ such enzymes as the KIenow fragment of DNA polymerase I, SEQUENASE (US Biochemical, Cleveland Ohio), Taq polymerase (Applied Biosystems), thermostable T7 polymerase (Amersham Pharmacia Biotech, Piscataway N.J.), or combinations of polymerases and proofreading exonucleases such as those found in the ELONGASE amplification system (Life Technologies, Gaithersburg Md.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the NEGABACE 1000 DNA sequencing system (Molecular Dynamics, Sunnyvale Calif.), or other systems known in the art. The resulting sequences are analyzed using a variety of algorithms which are well known in the art. (See, e.g., Ausubel, F. M. (1997) Short Protocols in Molecular Biologs, John Wiley & Sons, New York N.Y., unit 7.7; Meyers, R. A. (1995) Molecular Biology and Biotechnology, Wiley VCH, New York N.Y., pp. 856-853.)

The nucleic acid sequences encoding SECP may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector. (See, e.g., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomiclocus and surrounding sequences. (See, e.g., Triglia, T. et al. (1988) Nucleic Acids Res. 16:8186.) A third method, capture PCR, involves PCR amplification of DNA fragments adjacent to known sequences inhuman and yeast artificial chromosome DNA. (See, e.g., Lagerstrom, M. et al. (1991) PCR Methods Applic. 1:111-119.) In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For an PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotide sequences or fragments thereof which encode SECP may be cloned in recombinant DNA molecules that direct expression of SECP, or fragments or functional equivalents thereof, in appropriate host cells. Due to the inherent degeneracy of the genetic code, other DNA sequences which encode substantially the same or a functionally equivalent amino acid sequence may be produced and used to express SECP.

The nucleotide sequences of the present invention can be engineered using methods generally known in the art in order to alter SECP-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of SECP, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, sequences encoding SECP may be synthesized, in whole or in part, using chemical methods well known in the art. (See, e.g., Caruthers, M. H. et al. (1980) Nucleic Acids Symp. Ser. 7:215-223; and Horn, T. et al. (1980) Nucleic Acids Symp. Ser. 7:225-232.) Alternatively, SECP itself or a fragment thereof may be synthesized using chemical methods. For example, peptide synthesis can be performed using various solution-phase or solid-phase techniques. (See, e.g., Creighton, T. (1984) Proteins, Structures and Molecular Properties, WH Freeman, New York N.Y., pp. 55-60; and Roberge, J. Y. et al. (1995) Science 269:202-204.) Automated synthesis maybe achieved using the ABI 431A peptide synthesizer (Applied Biosystems). Additionally, the amino acid sequence of SECP, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography. (See, e.g., Chiez, R. M. and, F. Z. Regnier (1990) Methods Enzymol. 182:392-421.) The composition of the synthetic peptides may be confirmed by amino acid analysis or by sequencing. (See, e.g., Creighton, supra, pp. 28-53.)

In order to express a biologically active SECP, the nucleotide sequences encoding SECP or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotide sequences encoding SECP. Such elements may vary in their strength and specificity. Specific initiation signals may also be used to achieve more efficient translation of sequences encoding SECP. Such signals include the ATG initiation codon and adjacent sequences, e.g. the Kozak sequence. In cases where sequences encoding SECP and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results Probl. Cell Differ. 20:125-162.)

Methods which are well known to those skilled in the art may be used to construct expression vectors containing sequences encoding SECP and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York NY, ch. 9, 13, and 16.)

A variety of expression vector/host systems may be utilized to contain and express sequences encoding SECP. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. (See, e.g., Sambrook, supra; Ausubel, supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet. 15:345-355.) Expression vectors derived from retroviruses, adenoviruses, or herpes or vaccinia viruses, or from various bacterial plasmids, may be used for delivery of nucleotide sequences to the targeted organ, tissue, or cell population. (See, e.g., Di Nicola, M. et al. (1998) Cancer Gen Ther. 5(6):350-356; Yu, M. et al. (1993) Proc. Natl. Acad. Sci. USA 90(13):6340-6344; Buller, R. M. et al. (1985) Nature 317(6040):813-815; McGregor, D. P. et al. (1994) Mol. Immunol. 31(3):219-226; and Verma, I. M. and N. Somia (1997) Nature 389:239-242.) The invention is not limited by the host cell employed.

In bacterial systems, a number of cloning and expression vectors maybe selected depending upon the use intended for polynucleotide sequences encoding SECP. For example, routine cloning, subcloning, and propagation of polynucleotide sequences encoding SECP can be achieved using a multifunctional E. coli vector such as PBLUESCRIPT (Stratagene, La Jolla Calif.) or PSPORT1 plasmid (Life Technologies). Ligation of sequences encoding SECP into the vector's multiple cloning site disrupts the lacZ gene, allowing a calorimetric screening procedure for identification of transformed bacteria containing recombinant molecules. In addition, these vectors may be useful for in vitro transcription, dideoxy sequencing, single strand rescue with helper phage, and creation of nested deletionsin the cloned sequence. (See, e.g., Van Heeke, G. and S. M. Schuster (1989) J. Biol. Chem. 264:5503-5509.) When large quantities of SECP are needed, e.g. for the production of antibodies, vectors which direct high level expression of SECP may be used. For example, vectors containing the strong, inducible SP6 or T7 bacteriophage promoter may be used.

Yeast expression systems may be used for production of SECP. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign sequences into the host genome for stable propagation. (See, e.g., Ausubel, 1995, supra; Bitter, G. A. et al. (1987) Methods Enzymol. 153:516-544; and Scorer, C. A. et al. (1994) Bio/Technology 12:181-184.)

Plant systems may also be used for expression of SECP. Transcription of sequences encoding SECP may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. (See, e.g., The McGraw Hill Yearbook of Science and Technology (1992) McGraw Hill, New York N.Y., pp. 191-196.)

In mammalian cells, a number of viral-based expression systems may be utilized. In cases where an adenovirus is used as an expression vector, sequences encoding SECP may be ligated into an adenovinus transcription/translation complex consisting of the late promoter and tripartite leader sequence. Insertion in a non-essential E1 or E3 region of the viral genome may be used to obtain infective virus which expresses SECP in host cells. (See, e.g., Logan, J. and T. Shenk (1984) Proc. Natl. Acad. Sci. USA 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes. (See, e.g., Harrington, J. J. et al. (1997) Nat. Genet 15:345-355.)

For long term production of recombinant proteins in mammalian systems, stable expression of SECP in cell lines is preferred. For example, sequences encoding SECP can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively. (See, e.g., Wigler, M. et al. (1977) Cell 11:223-232; Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively. (See, e.g., Wigler, M. et al. (1980) Proc. Natl. Acad. Sci. USA 77:3567-3570; Colbere-Garapin, F. et al. (1981) J. Mol. Biol. 150:1-14.) Additional selectable genes have been described, e.g., trpB and hisD, which alter cellular requirements for metabolites. (See, e.g., Hartn, S. C. and R. C. Mulligan (1988) Proc. Natl. Acad. Sci. USA 85:8047-8051.) Visible markers, e.g., anthocyanins, green fluorescent proteins (GFP; Clontech), β glucuronidase and its substrate β-glucuronide, or luciferase and its substrate luciferin may be used. These markers can be used not only to identify transformants, but also to quantify the amount of transient or stable protein expression attributable to a specific vector system. (See, e.g., Rhodes, C. A. (1995) Methods Mol. Biol. 55:121-131.)

Although the presence/absence of marker gene expression suggests that the gene of interest is also present, the presence and expression of the gene may need to be confirmed. For example, if the sequence encoding SECP is inserted within a marker gene sequence, transformed cells containing sequences encoding SECP can be identified by the absence of marker gene function. Alternatively, a marker gene can be placed in tandem with a sequence encoding SECP under the control of a single promoter. Expression of the marker gene in response to induction or selection usually indicates expression of the tandem gene as well.

In general, host cells that contain the nucleic acid sequence encoding SECP and that express SECP may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of SECP using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on SECP is preferred, but a competitive binding assay may be employed. These and other assays are well known in the art. (See, e.g., Hampton, R. et al. (1990) Serological Methods, a Laboratory Manual, APS Press, St. Paul Minn., Sect IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.)

A wide variety of labels and conjugation techniques are known by those skilled in the art and may be used in various nucleic acid and amino acid assays. Means for producing labeled hybridization or PCR probes for detecting sequences related to polynucleotides encoding SECP include oligolabeling, nick translation, end-labeling, or PCR amplification using a labeled nucleotide. Alternatively, the sequences encoding SECP, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Pharmacia Biotech, Promega (Madison Wis.), and US Biochemical. Suitable reporter molecules or labels which maybe used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding SECP may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode SECP may be designed to contain signal sequences which direct secretion of SECP through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI3 8) are available from the American Type Culture Collection (ATCC, Manassas Va.) and may be chosen to ensure the correct modification and processing of the foreign protein.

In another embodiment of the invention, natural, modified, or recombinant nucleic acid sequences encoding SECP may be ligated to a heterologous sequence resulting in translation of a fusion protein in any of the aforementioned host systems. For example, a chimeric SECP protein containing a heterologous moiety that can be recognized by a commercially available antibody may facilitate the screening of peptide libraries for inhibitors of SECP activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodlin, and metal-chelate resins, respectively. FLAG, c-nyc, and hemagglutinin (HA) enable immunoaffimity purification of fusion proteins using commercially available monoclonal and polyclonal antibodies that specifically recognize these epitope tags. A fusion protein may also be engineered to contain a proteolytic cleavage site located between the SECP encoding sequence and the heterologous protein sequence, so that SECP may be cleaved away from the heterologous moiety following purification. Methods for fusion protein expression and purification are discussed in Ausubel (1995, supra, ch. 10). A variety of commercially available kits may also be used to facilitate expression and purification of fusion proteins.

In a further embodiment of the invention, synthesis of radiolabeled SECP may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

SECP of the present invention or fragments thereof may be used to screen for compounds that specifically bind to SECP. At least one and up to a plurality of test compounds may be screened for specific binding to SECP. Examples of test compounds include antibodies, oligonucleotides, proteins (e.g., receptors), or small molecules.

In one embodiment, the compound thus identified is closely related to the natural ligand of SECP, e.g., a ligand or fragment thereof, a natural substrate, a structural or functional mimetic, or a natural binding partner. (See, e.g., Coligan, J. E. et al. (1991) Current Protocols in Immunology 1(2): Chapter 5.) Similarly, the compound can be closely related to the natural receptor to which SECP binds, or to at least a fragment of the receptor, e.g., the ligand binding site. In either case, the compound can be rationally designed using known techniques. In one embodiment, screening for these compounds involves producing appropriate cells which express SECP, either as a secreted protein or on the cell membrane. Preferred cells include cells from mammals, yeast, Drosophila, or E. coli. Cells expressing SECP or cell membrane fractions which contain SECP are then contacted with a test compound and binding, stimulation, or inhibition of activity of either SECP or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with SECP, either in solution or affixed to a solid support, and detecting the binding of SECP to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

SECP of the present invention or fragments thereof maybe used to screen for compounds that modulate the activity of SECP. Such compounds may include agonists, antagonists, or partial or inverse agonists. In one embodiment, an assay is performed under conditions permissive for SECP activity, wherein SECP is combined with at least one test compound, and the activity of SECP in the presence of a test compound is compared with the activity of SECP in the absence of the test compound. A change in the activity of SECP in the presence of the test compound is indicative of a compound that modulates the activity of SECP. Alternatively, a test compound is combined with an in vitro or cell-free system comprising SECP under conditions suitable for SECP activity, and the assay is performed. In either of these assays, a test compound which modulates the activity of SECP may do so indirectly and need not come in direct contact with the test compound. At least one and up to a plurality of test compounds may be screened.

In another embodiment, polynucleotides encoding SECP or their mammalian homologs may be “knocked out” in an animal model system using homologous recombination in embryonic stem (ES) cells. Such techniques are well known in the art and are useful for the generation of animal models of human disease. (See, e.g., U.S. Pat. No. 5,175,383 and U.S. Pat. No. 5,767,337.) For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capeccbi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding SECP may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding SECP can also be used to create “knockin” humanized animals (pigs) or transgenic animals (mice or rats) to model human disease. With knockin technology, a region of a polynucleotide encoding SECP is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress SECP, e.g., by secreting SECP in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of SECP and secreted proteins. In addition, the expression of SECP is closely associated with diseased breast, brain, brain tumor, diseased brain, nervous, developmental, and diseased endometrial tissues, bone cancer, adrenal and brain tissues, and with neighboring tissues associated with tumors of the lung and ovary, dendritic cells, and kidney cortex tissue associated with a renal cancinoma, nasal, cribriform and ovarian tumor tissues. In addition, examples of tissues expressing SECP can be found in Table 6. Therefore, SECP appears to play a role in liver, cell proliferative, autoimmune/inflammatory, cardiovascular, neurological, and developmental disorders. In the treatment of disorders associated with increased SECP expression or activity, it is desirable to decrease the expression or activity of SECP. In the treatment of disorders associated with decreased SECP expression or activity, it is desirable to increase the expression or activity of SECP.

Therefore, in one embodiment, SECP or a fragment or derivative thereof may be administered to a subject to treat or prevent a disorder associated with decreased expression or activity of SECP. Examples of such disorders include, but are not limited to, a liver disorder such as adenomatosis, cholestasis, cirrhosis, hemangioma, Henoch-Schonlein purpura, hepatitis, hepatocellular and metastatic carcinomas, idiopathic thrombocytopenic purpura, porphyria, sarcoidosis, and Wilson disease and for diagnosis of liver disorders and detecting metabolic and toxicological responses to treatment with steroids; a cell proliferative disorder such as actinickeratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erylbroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenicpurpura, ulcerative colitis, uveitis, Werner syndrome, complications of cancer, hemodialysis, and extracorporeal circulation, viral, bacterial, fungal, parasitic, protozoal, and helminthic infections, and trauma; a cardiovascular disorder such as congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, complications of cardiac transplantation, arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; and a developmental disorder such as renal tubular acidosis, anemia, Cushing's syndrome, achondroplastic dwarfism Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss.

In another embodiment, a vector capable of expressing SECP or a fragment or derivative thereof may be administered to a subject, to treat or prevent a disorder associated with decreased expression or activity of SECP including, but not limited to, those described above.

In a further embodiment, a composition comprising a substantially purified SECP in conjunction with a suitable pharmaceutical carrier may be administered to a subject to treat or prevent a disorder associated with decreased expression or activity of SECP including, but not limited to, those provided above.

In still another embodiment, an agonist which modulates the activity of SECP may be administered to a subject to treat or prevent a disorder associated with decreased expression or activity of SECP including, but not limited to, those listed above.

In a further embodiment, an antagonist of SECP may be administered to a subject to treat or prevent a disorder associated with increased expression or activity of SECP. Examples of such disorders include, but are not limited to, those liver, cell proliferative, autoimmune/inflammatory, cardiovascular, neurological, and developmental disorders described above. In one aspect, an antibody which specifically binds SECP may be used directly as an antagonist or indirectly as a targeting or delivery mechanism for bringing a pharmaceutical agent to cells or tissues which express SECP.

In an additional embodiment, a vector expressing the complement of the polynucleotide encoding SECP may be administered to a subject to treat or prevent a disorder associated with increased expression or activity of SECP including, but not limited to, those described above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects. An antagonist of SECP may be produced using methods which are generally known in the art. In particular, purified SECP may be used to produce antibodies or to screen libraries of pharmaceutical agents to identify those which specifically bind SECP. Antibodies to SECP may also be generated using methods that are well known in the art. Such antibodies may include, but are not limited to, polygonal, monoclonal, chimeric, and single chain antibodies, Fab fragments, and fragments produced by a Fab expression library. Neutralizing antibodies (i.e., those which inhibit dimer formation) are generally preferred for therapeutic use. Single chain antibodies (e.g., from camels or llamas) may be potent enzyme inhibitors and may have advantages in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with SECP or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to SECP have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of SECP amino acids may be fused with those of another protein, such as KLH, and antibodies to the chimeric molecule may be produced.

Monoclonal antibodies to SECP may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-454.) Alternatively, techniques described for the production of single chain antibodies may be adapted, using methods known in the art, to produce SECP-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments which contain specific binding sites for SECP may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between SECP and its specific antibody. A two-site, monoclonal-based immunoassay utilizing monoclonal antibodies reactive to two non-interfering SECP epitopes is generally used, but a competitive binding assay may also be employed (Pound, supra).

Various methods such as Scatchard analysis in conjunction with radioimmunoassay techniques may be used to assess the affinity of antibodies for SECP. Affinity is expressed as an association constant, Kₐ, which is defined as the molar concentration of SECP-antibody complex divided by the molar concentrations of free antigen and free antibody under equilibrium conditions. The Kₐ determined for a preparation of polyclonal antibodies, which are heterogeneous in their affinities for multiple SECP epitopes, represents the average affinity, or avidity, of the antibodies for SECP. The Kₐ determined for a preparation of monoclonal antibodies, which are monospecific for a particular SECP epitope, represents a true measure of affinity. High-affinity antibody preparations with Kₐ ranging from about 10⁹ to 10¹² L/mole are preferred for use in immunoassays in which the SECP-antibody complex must withstand rigorous manipulations. Low-affinity antibody preparations with Kₐ ranging from about 10⁶ to 10⁷ L/mole are preferred for use in immunopurification and similar procedures which ultimately require dissociation of SECP, preferably in active form, from the antibody (Catty, D. (1988) Antibodies, Volume I: A Practical Approach, IRL Press, Washington DC; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of SECP-antibody complexes. Procedures for evaluating antibody specificity, titer, and avidity, and guidelines for antibody quality and usage in various applications, are generally available. (See, e.g., Catty, supra, and Coligan et al. supra.)

In another embodiment of the invention, the polynucleotides encoding SECP, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding SECP. Such technology is well known in the art, and antisense oligonucleotides or larger fragments can be designed from various locations along the coding or control regions of sequences encoding SECP. (See, e.g., Agrawal, S., ed. (1996) Antisense Therapeutics, Humana Press Inc., Totawa N.J.)

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein. (See, e.g., Slater, J. E. et al. (1998) J. Allergy Clin. Immunol. 102(3):469-475; and Scanlon, K. J. et al. (1995) 9(13):1288-1296.) Antisense sequences can also be introduced intracellularly through the use of viral vectors, such as retrovirus and adeno-associated virus vectors. (See, e.g., Miller, A. D. (1990) Blood 76:271; Ausubel, supra; Uckert, W. and W. Walther (1994) Pharmacol. Ther. 63(3):323-347.) Other gene delivery mechanisms include liposome-derived systems, artificial viral envelopes, and other systems known in the art. (See, e.g., Rossi, J. J. (1995) Br. Med. Bull. 51(1):217-225; Boado, R. J. et al. (1998) J. Pharm. Sci. 87(11):1308-1315; and Morris, M. C. et al. (1997) Nucleic Acids Res. 25(14):2730-2736.)

In another embodiment of the invention, polynucleotides encoding SECP may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in SECP expression or regulation causes disease, the expression of SECP from an appropriate population of transduced cells may alleviate the clinical manifestations caused by the genetic deficiency.

In a further embodiment of the invention, diseases or disorders caused by deficiencies in SECP are treated by constructing mammalian expression vectors encoding SECP and introducing these vectors by mechanical means into SECP-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem 62:191-217; Ivics, Z. (1997) Cell 91:501-510; Boulay, J-L. and H Récipon (1998) Curr. Opin. Biotechnol. 9:445-450).

Expression vectors that may be effective for the expression of SECP include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). SECP may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding SECP from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to SECP expression are treated by constructing a retrovirus vector consisting of (i) the polynucleotide encoding SECP under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In the alternative, an adenovirus-based gene therapy delivery system is used to deliver polynucleotides encoding SECP to cells which have one or more genetic abnormalities with respect to the expression of SECP. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999) Annu. Rev. Nutr. 19:511-544 and Verna, I. M. and N. Somia (1997) Nature 18:389:239-242, both incorporated by reference herein.

In another alternative, a herpes-based, gene therapy delivery system is used to deliver polynucleotides encoding SECP to target cells which have one or more genetic abnormalities with respect to the expression of SECP. The use of herpes simplex virus (HSV)-based vectors may be especially valuable for introducing SECP to cells of the central nervous system, for which HSV has a tropism The construction and packaging of herpes-based vectors are well known to those with ordinary skill in the art. A replication-competent herpes simplex virus (HSV) type l-based vector has been used to deliver a reporter gene to the eyes of primates (Liu, X et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999) J. Virol. 73:519-532 and Xu, H. et al. (1994) Dev. Biol. 163:152-161, hereby incorporated by reference. The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another alternative, an alphavirus (positive, single-stranded RNA virus) vector is used to deliver polynucleotides encoding SECP to target cells. The biology of the prototypic alphavirus, Semliki Forest Virus (SFV), has been studied extensively and gene transfer vectors have been based on the SFV genome (Garoff, H. and K-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for SECP into the alphavirus genome in place of the capsid-coding region results in the production of a large number of SECP-coding RNAs and the synthesis of high levels of SECP in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alpha viruses will allow the introduction of SECP into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing, Mt. Kisco N.Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of sequences encoding SECP.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis:. Alternatively, RNA molecules may be generated by in vitro and in vivo transcription of DNA sequences encoding SECP. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding SECP. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased SECP expression or activity, a compound which specifically inhibits expression of the polynucleotide encoding SECP may be therapeutically useful, and in the treatment of disorders associated with decreased SECP expression or activity, a compound which specifically promotes expression of the polynucleotide encoding SECP may be therapeutically useful.

At least one, and up to a plurality, of test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding SECP is exposed to at least one test compound thus obtained. The sample may comprise, for example, an intact or permeabilized cell, or an in vitro cell-free or reconstituted biochemical system Alterations in the expression of a polynucleotide encoding SECP are assayed by any method commonly known in the art. Typically, the expression of a specific nucleotide is detected by hybridization with a probe having a nucleotide sequence complementary to the sequence of the polynucleotide encoding SECP. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient Delivery by transfection, by liposome injections, or by polycationic amino polymers may be achieved using methods which are well known in the art. (See, e.g., Goldman, C. K. et al. (1997) Nat Biotechnol. 15:462-466.)

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient Excipients may include, for example, sugars, starches, celluloses, gums, and proteins. Various formulations are commonly known and are thoroughly discussed in the latest edition of Remingon's Pharmaceutical Sciences (Maack Publishing, Easton Pa.). Such compositions may consist of SECP, antibodies to SECP, and mimetics, agonists, antagonists, or inhibitors of SECP.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising SECP or fragments thereof. For example, liposome preparations containing a cell-impermeable macromolecule may promote cell fusion and intracellular delivery of the macromolecule. Alternatively, SECP or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example SECP or fragments thereof, antibodies of SECP, and agonists, antagonists or inhibitors of SECP, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind SECP may be used for the diagnosis of disorders characterized by expression of SECP, or in assays to monitor patients being treated with SECP or agonists, antagonists, or inhibitors of SECP. Antibodies useful for diagnostic purposes may be prepared in the same manner as described above for therapeutics. Diagnostic assays for SECP include methods which utilize the antibody and a label to detect SECP in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring SECP, including ELISAs, RIAS, and FACS, are known in the art and provide a basis for diagnosing altered or abnormal levels of SECP expression. Normal or standard values for SECP expression are established by combining body fluids or cell extracts taken from normal mammalian subjects, for example, human subjects, with antibodies to SECP under conditions suitable for complex formation. The amount of standard complex formation may be quantitated by various methods, such as photometric means. Quantities of SECP expressed in subject, control, and disease samples from biopsied tissues are compared with the standard values. Deviation between standard and subject values establishes the parameters for diagnosing disease.

In another embodiment of the invention, the polynucleotides encoding SECP may be used for diagnostic purposes. The polynucleotides which may be used include oligonucleotide sequences, complementary RNA and DNA molecules, and PNAs. The polynucleotides may be used to detect and quantify gene expression in biopsied tissues in which expression of SECP may be correlated with disease. The diagnostic assay may be used to determine absence, presence, and excess expression of SECP, and to monitor regulation of SECP levels during therapeutic intervention.

In one aspect, hybridization with PCR probes which are capable of detecting polynucleotide sequences, including genomic sequences, encoding SECP or closely related molecules may be used to identify nucleic acid sequences which encode SECP. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding SECP, allelic variants, or related sequences.

Probes may also be used for the detection of related sequences, and may have at least 50% sequence identity to any of the SECP encoding sequences. The hybridization probes of the subject invention may be DNA or RNA and may be derived from the sequence of SEQ ID NO:31-60 or from genomic sequences including promoters, enhancers, and introns of the SECP gene.

Means for producing specific hybridization probes for DNAs encoding SECP include the cloning of polynucleotide sequences encoding SECP or SECP derivatives into vectors for the production of mRNA probes. Such vectors are known in the art, are commercially available, and maybe used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotide sequences encoding SECP may be used for the diagnosis of disorders associated with expression of SECP. Examples of such disorders include, but are not limited to, a liver disorder such as adenomatosis, cholestasis, cirrhosis, hemangioma, Henoch-Schonlein purpura, hepatitis, hepatocellular and metastatic carcinomas, idiopathic thrombocytopenic purpura, porphyria, sarcoidosis, and Wilson disease and for diagnosis of liver disorders and detecting metabolic and toxicological responses to treatment with steroids; a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid artritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a cardiovascular disorder such as congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, complications of cardiac transplantation, arteriovenous fistula, atherosclerosis, hypertension, vasculitis, Raynaud's disease, aneurysms, arterial dissections, varicose veins, thrombophlebitis and phlebothromnbosis, vascular tumors, and complications of thrombolysis, balloon angioplasty, vascular replacement, and coronary artery bypass graft surgery; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; and a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss. The polynucleotide sequences encoding SECP may be used in Southern or northern analysis, dot blot, or other membrane-based technologies; in PCR technologies; in dipstick, pin, and multiformat ELISA-like assays; and in microarrays utilizing fluids or tissues from patients to detect altered SECP expression. Such qualitative or quantitative methods are well known in the art.

In a particular aspect, the nucleotide sequences encoding SECP may be useful in assays that detect the presence of associated disorders, particularly those mentioned above. The nucleotide sequences encoding SECP may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of nucleotide sequences encoding SECP in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of SECP, a normal or standard profile for expression is established. This may be accomplished by combining body fluids or cell extracts taken from normal subjects, either animal or human, with a sequence, or a fragment thereof, encoding SECP, under conditions suitable for hybridization or amplification. Standard hybridization may be quantified by comparing the values obtained from normal subjects with values from an experiment in which a known amount of a substantially purified polynucleotide is used. Standard values obtained in this maimer may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding SECP may involve the use of PCR These oligomers may be chemically synthesized, generated enzymatically, or produced in vitro. Oligomers will preferably contain a fragment of a polynucleotide encoding SECP, or a fragment of a polynucleotide complementary to the polynucleotide encoding SECP, and will be employed under optimized conditions for identification of a specific gene or condition. Oligomers may also be employed under less stringent conditions for detection or quantification of closely related DNA or RNA sequences.

In a particular aspect, oligonucleotide primers derived from the polynucleotide sequences encoding SECP may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from the polynucleotide sequences encoding SECP are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the ampliers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations. (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641.)

Methods which may also be used to quantify the expression of SECP include radiolabeling or biotinylating nucleotides, coamplification of a control nucleic acid, and interpolating results from standard curves. (See, e.g., Melby, P. C. et al. (1993)J. Immunol. Methods 159:235-244; Duplaa, C. et al. (1993) Anal. Biochem. 212:229-236.) The speed of quantitation of multiple samples may be accelerated by running the assay in a high-throughput format where the oligomer or polynucleotide of interest is presented in various dilutions and aspectrophotometric or colorimetric response gives rapid quantitation.

In further embodiments, oligonucleotides or longer fragments derived from any of the polynucleotide sequences described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient For example, therapeutic agents which are highly effective and display the fewest side effects may be selected for a patient based on his/her pharmacogenomic profile.

In another embodiment, SECP, fragments of SECP, or antibodies specific for SECP may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time. (See Seilkamer et al., “Comparative Gene Transcript Analysis,” U.S. Pat. No. 5,840,484, expressly incorporated by reference herein.) Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471, expressly incorporated by reference herein). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity. (See, for example, Press Release 00-02 from the National Institute of Environmental Health Sciences, released February 29, 2000, available at http://www.niehs.nih.gov/oc/news/toxchip.ht) Therefore, it is important and desirable in toxicological screening using toxicant signatures to include all expressed gene sequences.

In one embodiment, the toxicity of a test compound is assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another particular embodiment relates to the use of the polypeptide sequences of the present invention to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of the present invention. In some cases, further sequence data may be obtained for definitive protein identification.

A proteomic profile may also be generated using antibodies specific for SECP to quantify the levels of SECP expression. In one embodiment, the antibodies are used as elements on a microarray, and protein expression levels are quantified by exposing the microarray to the sample and detecting the levels of protein bound to each array element (Lueking, A. et al. (1999) Anal. Biochem 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.) Various types of microarrays are well known and thoroughly described in DNA Microarrays: A Practical Approach, M. Schena, ed. (1999) Oxford University Press, London, hereby expressly incorporated by reference.

In another embodiment of the invention, nucleic acid sequences encoding SECP may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (199 1) Trends Genet. 7:149-154.) Once mapped, the nucleic acid sequences of the invention may be used to develop genetic linkage maps, for example, which correlate the inheritance of a disease state with the inheritance of a particular chromosome region or restriction fragment length polymorphism (RFLP). (See, for example, Lander, E. S. and D. Botstein (1986) Proc. Natl. Acad. Sci. USA 83:7353-7357.)

Fluorescent in situ hybridization (FISH) may be correlated with other physical and genetic map data. (See, e.g., Heinz-Ulrich, et al. (1995) in Meyers, supra, pp. 965-968.) Examples of genetic map data can be found in various scientific journals or at the Online Mendelian Inheritance in Man (OMIM) World Wide Web site. Correlation between the location of the gene encoding SECP on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequence of the instant invention may also be used to detect differences in the chromosomal location due to translocation, inversion, etc., among normal, carrier, or affected individuals.

In another embodiment of the invention, SECP, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between SECP and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate. The test compounds are reacted with SECP, or fragments thereof, and washed. Bound SECP is then detected by methods well known in the art. Purified SECP can also be coated directly onto plates for use in the aforementioned drug screening techniques. Alternatively, non-neutraling antibodies can be used to capture the peptide and immobilize it on a solid support.

In another embodiment, one may use competitive drug screening assays in which neutralizing antibodies capable of binding SECP specifically compete with a test compound for binding SECP. In this manner, antibodies can be used to detect the presence of any peptide which shares one or more antigenic determinants with SECP.

In additional embodiments, the nucleotide sequences which encode SECP may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent The following preferred specific embodiments are, therefore, to be construed as merely illustrative, and not limitative of the remainder of the disclosure in any way whatsoever.

The disclosures of all patents, applications, and publications mentioned above and below, including U.S. Ser. No. 60/285,207, U.S. Ser. No. 60/287,114, U.S. Ser. No. 60/288,640, U.S. Ser. No. 60/290,516, U.S. Ser. No. 60/292,184, U.S. Ser. No. 60/343,553, U.S. Ser. No. 60/358,279, U.S. Ser. No. 60/366,041, and U.S. Ser. No. 60/357,002, are hereby expressly incorporated by reference.

### EXAMPLES

I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Life Technologies), a monophasic solution of phenol and guanidine isothiocyanate. The resulting lysates were centrifuged over CsCl cushions or extracted with chloroform RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Life Technologies), using the recommended procedures or similar methods known in the art. (See, e.g., Ausubel, 1997, supra, units 5.1-6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Pharmacia Biotech) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Life Technologies), PCDNA2.1 plasmid (Invitrogen, Carlsbad Calif.), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof Recombinant plasmids were transformed into competent E. coli cells including XL1-Blue, XL1-BlueMRF, or SOLR from Stratagene or DH5α, DH10B, or ElectroMAX DH10B from Life Technologies.

II. Isolation of cDNA Clones

Plasmids obtained as described in Example I were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Pharmacia Biotech or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Molecular Dynamics); the ABI PRISM 373 or 377 sequencing system (Applied Biosystems) in conjunction with standard ABI protocols and base calling software; or other sequence analysis systems known in the art. Reading frames within the cDNA sequences were identified using standard methods (reviewed in Ausubel, 1997, supra, unit 7.7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosaccharomyces pombe, and Candida albicans (Incyte Genomics, Palo Alto Calif.); hidden Markov model (HMM)-based protein family databases such as PFAM, INCY, and TIGRFAM (Haft, D. H. et al. (2001) Nucleic Acids Res. 29:41-43); and HMM-based protein domain databases such as SMART (Schultz et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families. See, for example, Eddy, S. R (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and PASTA. The full length polynucleotide sequences were translated to derive the corresponding fill length polypeptide sequences. Alternatively, a polypeptide of the invention may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRPAM; and HMM-based protein domain databases such as SMART. Full length polynucleotide sequences are also analyzed using MACDNASIS PRO software (Hitachi Software Engineering, South San Francisco Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:31-60. Fragments from about 20 to about 4000 nucleotides which are useful in hybridization and amplification technologies are described in Table 4, column 2.

IV. Identification and Editing of Coding Sequences from Genomic DNA

Putative secreted proteins were initially identified by running the Genscan gene identification program against public genomic sequence databases (e.g., gbpri and gbhtg). Genscan is a general-purpose gene identification program which analyzes genomic DNA sequences from a variety of organisms (See Burge, C. and S. Karlin (1997) J. Mol. Biol. 268:78-94, and Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode secreted proteins, the encoded polypeptides were analyzed by querying against PFAM models for secreted proteins. Potential secreted proteins were also identified by homology to Incyte cDNA sequences thathadbeen annotated as secreted proteins. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this informationwas used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example III were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example III were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of SECP Encoding Polynucleotides

The sequences which were used to assemble SEQ ID NO:31-60 were compared with sequences from the Incyte LIFESEQ database and public domain databases using BLAST and other implementations of the Smith-Waterman algorithm Sequences from these databases that matched SEQ ID NO:31-60 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Genethon were used to determine if any of the clustered sequences had been previously mapped. Inclusion of a mapped sequence in a cluster resulted in the assignment of all sequences of that cluster, including its particular SEQ ID NO:, to that map location

Map locations are represented by ranges, or intervals, of human chromosomes. The map position of an interval, in centiMorgans, is measured relative to the terminus of the chromosome's p-arm (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Genethon which provide boundaries for radiation hybrid markers whose sequences were included in each of the clusters. Human genome maps and other resources available to the public, such as the NCBI “GeneMap '99” World Wide Web site (http://www.ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel (1995) supra, ch. 4 and 16.)

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced eitherby 100% identity and 50% overlap at one end, or 79% identity and 100% overlap.

Alternatively, polynucleotide sequences encoding SECP are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system;liver; musculoskeletal system; nervous system; pancreas; respiratory system; sense organs; skin; stomatognathic system; unclassified/mixed; or urinary tract The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding SECP. cDNA sequences and cDNA library/tissue information are found in the LIFESEQ GOLD database (Incyte Genomics, Palo Alto Calif.).

VIII. Extension of SECP Encoding Polynucleotides

Full length polynucleotide sequences were also produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68 ° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68 ° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1× TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Pharmacia Biotech). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Pharmacia Biotech), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carb liquid media.

The cells were lysed, and DNA was amplified by PCR using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Pharmacia Biotech) or the ABI PRISM BIGDYE Terminator cycle sequencing ready reaction kit (Applied Biosystems).

In like manner, full length polynucleotide sequences are verified using the above procedure or are used to obtain 5′ regulatory sequences using the above procedure along with oligonucleotides designed for such extension, and an appropriate genomic library.

IX. Identification of Single Nucleotide Polymorphisms in SECP Encoding Polynucleotides

Common DNA sequence variants known as single nucleotide polymorphisms (SNPs) were identified in SEQ ID NO:31-60 using the LIFESEQ database (ncyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31%Japanese, 13% Korean,5% Vietnamese, and 8% other Asian. Allele frequencies were first analyzed in the Caucasian population; in some cases those SNPs which showed no allelic variance in this population were not further tested in the other three populations.

X. Labeling and Use of Individual Hybridization Probes

Hybridization probes derived from SEQ ID NO:31-60 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Pharmacia Biotech), and T4 polynucleotide kinase (DuPont NEN, Boston Mass.). The labeled oligonucleotides are substantially purified using a SEPHADEX G-25 superfine size exclusion dextran bead column (Amersham Pharmacia Biotech). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Scbleicher & Schuell, Durham N.H.). Hybridization is carried out for 16 hours at 40° C. To remove nonspecific signals, blots are sequentially washed at room temperature under conditions of up to, for example, 0.1×saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (ink-jet printing, See, e.g., Baldeschweiler, supra.), mechanical microspotting technologies, and derivatives thereof. The substrate in each of the aforementioned technologies should be uniform and solid with a non-porous surface (Schena (1999), supra). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements. (See, e.g., Schena, M. et al. (1995) Science 270:467-470; Shalon, D. et al. (1996) Genome Res. 6:639-645; Marshall, A. and J. Hodgson (1998) Nat. Biotechnol. 16:27-31.)

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorption and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Pharmacia Biotech). The reverse transcription reaction is performed in a 25 ml volume containing 200 ng poly(A)⁺ RNA with GEMBRIGHT kits (Incyte). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Pharmacia Biotech).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridizationbuffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with are solution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum

A grid is superimposed over the fluorescence signal image such that the signal from each spot is centered in each element of the grid. The fluorescence signal within each element is then integrated to obtain a numerical value corresponding to the average intensity of the signal. The software used for signal- analysis is the GEMTOOLS gene expression analysis program (Incyte).

Human THP-1 cells (American Type Culture Collection, Manassas Va.) were grown in RPMI1640 medium containing 10% fetal serum (v/v), 0.45% glucose (w/v), 10 mM Hepes, 1 mM sodium pyruvate, 1×10⁻⁵ M β-mercaptoethanol, penicillin (100 units/ml) and streptomycin (100 mg/ml). For oxidized-LDL loading experiments, cells were seeded at a density of 1×10⁶ cells/ml in medium containing 12-0-tetradecanoyl-phorbol-13-acetate (Research Biochemical International, Natick Mass.) at 1×10⁻⁷ M for 24 hr. The medium was then replaced by culture medium with or without 100 μg/ml of CuSO₄ “fully” oxidized LDL (Intracel, Rockville Md.) according to the method of Hammer et al. (1995; Arterio Thromb Vasc Biol 15:704-713). Medium was replaced every two days during the time of culture. Cells were treated with Ox-LDL over time points ranging from 30 minutes to 4 days. During this period, cells remained adherent and had a typical speckled Nile red staining pattern. RNA was prepared for expression profiling at 0, 0.5, 2.5, and 8 hours, and 1, 2, and 4 days of Ox-LDL exposure.

Steroid Treatment of Human Liver C3A Cells

Early confluent C3A cells were treated with mifepristone, progesterone, beclomethasone, medroxyprogesterone, budesonide, prednisone, dexamethasone, betamethasone, or danazol at concentrations of 1 μM, 10 μM, and 100 μM for 1, 3, and 6 hours. In all cases mRNA from untreated early confluent C3A cells were prepared in parallel.

In this way it was shown that for SEQ ID NO:31, SECP was downregulated at least 2-fold in three of six lung tumor tissues tested. In addition it was down-regulated at least 3-fold in a THP-1 model for foam cell formation. For SEQ ID NO:34, it was shown that SECP expression was up-regulated at least 2.5-fold in a C3A liver cell line when treated with the following steroid compounds: beclomethasone (Beclo), medroxyprogesterone (MAH), prednisone (Prdsne), dexamethasone (Dex), and with betamethas one (Betam).

XII. Complementary Polynucleotides

Sequences complementary to the SECP-encoding sequences, or any parts thereof, are used to detect, decrease, or inhibit expression of naturally occurring SECP. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of SECP. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the SECP-encoding transcript.

XIII. Expression of SECP

Expression and purification of SECP is achieved using bacterial or virus-based expression systems. For expression of SECP in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express SECP upon induction with isopropyl beta-D-thiogalactopyranoside (IPTG). Expression of SECP in eukaryotic cells is achieved by infecting insect or mammalian cell lines with recombinant Autoraphica californica nuclear polyhedrosis virus (AcMNPV), commonly known as baculovirus. The nonessential polyhedrin gene of baculovirus is replaced with cDNA encoding SECP by either homologous recombination or bacterial-mediated transposition involving transfer plasmid intermediates. Viral infectivity is maintained and the strong polyhedrin promoter drives high levels of cDNA transcription Recombinant baculovirus is used to infect Spodoptera frugiperda (Sf9) insect cells in most cases, or human hepatocytes, in some cases. Infection of the latter requires additional genetic. modifications to baculovirus. (See Engelhard, E. K. et al. (1994) Proc. Natl. Acad. Sci. USA 91:3224-3227; Sandig, V. et al. (1996) Hum. Gene Ther. 7:1937-1945.)

In most expression systems, SECP is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutatlione under conditions that maintain protein activity and antigenicity (Amersham Pharmacia Biotech). Following purification, the GST moiety can be proteolytically cleaved from SECP at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel (1995, supra, ch. 10 and 16). Purified SECP obtained by these methods can be used directly in the assays shown in Examples XVII, XVIII, and XIX, where applicable.

XV. Functional Assays

SECP function is assessed by expressing the sequences encoding SECP at physiologically elevated levels in mammalian cell culture systems. cDNA is subcloned into a mammalian expression vector containing a strong promoter that drives high levels of cDNA expression Vectors of choice include PCMV SPORT Wife Technologies) and PCR3.1 (Invitrogen, Carlsbad Calif.), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of SECP on gene expression can be assessed using highly purified populations of cells transfected with sequences encoding SECP and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding SECP and other genes of interest can be analyzed by northern analysis or microarray techniques.

XV. Production of SECP Specific Antibodies

SECP substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the SECP amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. (See, e.g., Ausubel, 1995, supra, ch 11.)

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity. (See, e.g., Ausubel, 1995, supra.) Rabbits are immunized with the oligopeptide-KLH complex in complete Freund's adjuvant. Resulting antisera are tested for antipeptide and anti-SECP activity by, for example, binding the peptide or SECP to a substrate, blocking with 1% BSA, reacting with rabbit antisera, washing, and reacting with radio-iodinated goat anti-rabbit IgG.

XVI. Purification of Naturally Occurring SECP Using Specific Antibodies

Naturally occurring or recombinant SECP is substantially purified by immunoaffinity chromatography using antibodies specific for SECP. An immunoaffinity column is constructed by covalently coupling anti-SECP antibody to an activated chromatographic resin, such as CNBr-activated SEPHAROSE (Amersham Pharmacia Biotech). After the coupling, the resin is blocked and washed according to the manufacturer's instructions.

Media containing SECP are passed over the immunoaffinity column, and the column is washed under conditions that allow the preferential absorbance of SECP (e.g., high ionic strength buffers in the presence of detergent). The column is eluted under conditions that disrupt antibody/SECP binding (e.g., a buffer of pH 2 to pH 3, or a high concentration of a chaotrope, such as urea or thiocyanate ion), and SECP is collected.

XVII. Identification of Molecules which Interact with SECP

SECP, or biologically active fragments thereof, are labeled with ¹²⁵I Bolton-Hunter reagent (See, e.g., Bolton, A. E. and W. M. Hunter (1973) Biochem. J. 133:529-539.) Candidate molecules previously arrayed in the wells of a multi-well plate are incubated with the labeled SECP, washed, and any wells with labeled SECP complex are assayed. Data obtained using different concentrations of SECP are used to calculate values for the number, affinity, and association of SECP with the candidate molecules.

Alternatively, molecules interacting with SECP are analyzed using the yeast two-hybrid system as described in Fields, S. and O. Song (1989) Nature 340:245-246, or using commercially available kits based on the two-hybrid system, such as the MATCHMAKER system (Clontech).

SECP may also be used in the PATHCALLING process (CuraGen Corp., New Haven Conn.) which employs the yeast two-hybrid system in a high-throughput manner to determine all interactions between the proteins encoded by two large libraries of genes (Nandabalan, K et al. (2000) U.S. Pat. No. 6,057,101).

XVIII. Demonstration of SECP Activity

An assay for growth stimulating or inhibiting activity of SECP measures the amount of DNA synthesis in Swiss mouse 3T3 cells (McKay, I. and Leigh, I., eds. (1993) Growth Factors: A Practical Approach, Oxford University Press, New York, N.Y.). In this assay, varying amounts of SECP are added to quiescent 3T3 cultured cells in the presence of [³H]thymidine, a radioactive DNA precursor. SECP for this assay can be obtained by recombinant means or from biochemical preparations. Incorporation of [³H]thymidine into acid-precipitable DNA is measured over an appropriate time interval, and the amount incorporated is directly proportional to the amount of newly synthesized DNA. A linear dose-response curve over at least a hundred-fold SECP concentration range is indicative of growth modulating activity. One unit of activity per milliliter is defined as the concentration of SECP producing a 50% response level, where 100% represents maxima incorporation of [³H]thymidine into acid-precipitable DNA.

Alternatively, an assay for SECP activity measures the stimulation or inhibition of neurotransmission in cultured cells. Cultured CHO fibroblasts are exposed to SECP. Following endocytic uptake of SECP, the cells are washed with fresh culture medium and a whole cell voltage-clamped Xenopus myocyte is manipulated into contact with one of the fibroblasts in SECP-free medium. Membrane currents are recorded from the myocyte. Increased or decreased current relative to control values are indicative of neuromodulatory effects of SECP (Morimoto, T. et al. (1995) Neuron 15:689-696).

Alternatively, an assay for SECP activity measures the amount of SECP in secretory, membrane-bound organelles. Transfected cells as described above are harvested and lysed. The lysate is fractionated using methods known to those of skill in the art, for example, sucrose gradient ultracentrifigation. Such methods allow the isolation of subcellular components such as the Golgi apparatus, ER, small membrane-bound vesicles, and other secretory organelles. Immunoprecipitations from fractionated and total cell lysates are performed using SECP-specific antibodies, and immunoprecipitated samples are analyzed using SDS-PAGE and immunoblotting techniques. The concentration of SECP in secretory organelles relative to SECP in total cell lysate is proportional to the amount of SECP in transit through the secretory pathway.

Alternatively, AMP binding activity is measured by combining SECP with ³²P-labeled AMP. The reaction is incubated at 37° C. and terminated by addition of tridcloroacetic acid. The acid extract is neutralized and subjected to gel electrophoresis to remove unbound label. The radioactivity retained in the gel is proportional to SECP activity.

XIX. Demonstration of Immunoglobulin Activity

An assay for SECP activity measures the ability of SECP to recognize and precipitate antigens from serum. This activity can be measured by the quantitative precipitin reaction. (Golub, E. S. et al. (1987) Immunology: A Synthesis, Sinauer Associates, Sunderland, Mass., pages 113-115.) SECP is isotopically labeled using methods known in the art. Various serum concentrations are added to constant amounts of labeled SECP. SECP-antigen complexes precipitate out of solution and are collected by centrifugation. The amount of precipitable SECP-antigen complex is proportional to the amount of radioisotope detected in the precipitate. The amount of precipitable SECP-antigen complex is plotted against the serum concentration. For various serum concentrations, a characteristic precipitin curve is obtained, in which the amount of precipitable SECP-antigen complex initially increases proportionately with increasing serum concentration, peaks at the equivalence point, and then decreases proportionately with further increases in serum concentration. Thus, the amount of precipitable SECP-antigen complex is a measure of SECP activity which is characterized by sensitivity to both limiting and excess quantities of antigen.

Alternatively, an assay for SECP activity measures the expression of SECP on the cell surface. cDNA encoding SECP is transfected into a non-leukocytic cell line. Cell surface proteins are labeled with biotin (de 1a Puente, M. A. et al. (1997) Blood 90:2398-2405). Immunoprecipitations are performed using SECP-specific antibodies, and immunoprecipitated samples are analyzed using SDS-PAGE and immunoblotting techniques. The ratio of labeled immunoprecipitant to unlabeled immunoprecipitant is proportional to the amount of SECP expressed on the cell surface.

Alternatively, an assay for SECP activity measures the amount of cell aggregation induced by overexpression of SECP. In this assay, cultured cells such as NIH3T3 are transfected with cDNA encoding SECP contained within a suitable mammalian expression vector under control of a strong promoter. Cotransfection with cDNA encoding a fluorescent marker protein, such as Green Huorescent Protein (CLONTECH), is useful for identifying stable transfectants. The amount of cell agglutination, or clumping, associated with transfected cells is compared with that associated with untransfected cells. The amount of cell agglutination is a direct measure of SECP activity.

Various modifications and variations of the described methods and systems of the invention will be apparent to those skilled in the art without departing from the scope and spirit of the invention. Although the invention has been described in connection with certain embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

TABLE 1

| | | | | Incyte | |
|---|---|---|---|---|---|
| | Polypeptide | Incyte | Polynucleotide | Polynucleotide | Incyte Full Length |
| Incyte Project ID | SEQ ID NO: | Polypeptide ID | SEQ ID NO: | ID | Clones |
| 1895273 | 1 | 1895273CD1 | 31 | 1895273CB1 | 1895273CA2 |
| 70072222 | 2 | 70072222CD1 | 32 | 70072222CB1 | 813820CA2 |
| 3559223 | 3 | 3559223CD1 | 33 | 3559223CB1 | |
| 3441255 | 4 | 3441255CD1 | 34 | 3441255CB1 | |
| 1958917 | 5 | 1958917CD1 | 35 | 1958917CB1 | 90067353CA2 |
| 6219465 | 6 | 6219465CD1 | 36 | 6219465CB1 | |
| 3576625 | 7 | 3576625CD1 | 37 | 3576625CB1 | |
| 4765758 | 8 | 4765758CD1 | 38 | 4765758CB1 | 4765758CA2 |
| 7236661 | 9 | 7236661CD1 | 39 | 7236661CB1 | 6764860CA2, |
| | | | | | 6772618CA2, |
| | | | | | 7236661CA2 |
| 7714187 | 10 | 7714187CD1 | 40 | 7714187CB1 | |
| 5136540 | 11 | 5136540CD1 | 41 | 5136540CB1 | 5136540CA2 |
| 3277403 | 12 | 3277403CD1 | 42 | 3277403CB1 | 90093289CA2, |
| | | | | | 90097965CA2 |
| 1517569 | 13 | 1517569CD1 | 43 | 1517569CB1 | |
| 2415991 | 14 | 2415991CD1 | 44 | 2415991CB1 | 4031777CA2, |
| | | | | | 7383971CA2 |
| 2735742 | 15 | 2735742CD1 | 45 | 2735742CB1 | 1842245CA2, |
| | | | | | 1862749CA2, |
| | | | | | 2765810CA2, |
| | | | | | 3174585CA2, |
| | | | | | 6034213CA2, |
| | | | | | 6361652CA2, |
| | | | | | 7638315CA2, |
| | | | | | 7977239CA2, |
| | | | | | 8087951CA2, |
| | | | | | 8094152CA2, |
| | | | | | 8250338CA2, |
| | | | | | 8531020CA2, |
| | | | | | 8743705CA2 |
| 2768535 | 16 | 2768535CD1 | 46 | 2768535CB1 | 4239666CA2 |
| 6848851 | 17 | 6848851CD1 | 47 | 6848851CB1 | |
| 7040722 | 18 | 7040722CD1 | 48 | 7040722CB1 | 56022479CA2 |
| 6430290 | 19 | 6430290CD1 | 49 | 6430290CB1 | 90076008CA2, |
| | | | | | 90076371CA2, |
| | | | | | 90076387CA2, |
| | | | | | 90076395CA2 |
| 2640251 | 20 | 2640251CD1 | 50 | 2640251CB1 | 6452666CA2, |
| | | | | | 8516219CA2 |
| 3839350 | 21 | 3839350CD1 | 51 | 3839350CB1 | 3839350CA2 |
| 6393813 | 22 | 6393813CD1 | 52 | 6393813CB1 | 1615668CA2, |
| | | | | | 2193894CA2, |
| | | | | | 6557563CA2, |
| | | | | | 6557564CA2, |
| | | | | | 7057161CA2 |
| 5685755 | 23 | 5685755CD1 | 53 | 5685755CB1 | |
| 71728459 | 24 | 71728459CD1 | 54 | 71728459CB1 | 5405225CA2 |
| 1904303 | 25 | 1904303CD1 | 55 | 1904303CB1 | |
| 2911343 | 26 | 2911343CD1 | 56 | 2911343CB1 | 90094777CA2, |
| | | | | | 90094885CA2 |
| 7500308 | 27 | 7500308CD1 | 57 | 7500308CB1 | 2343465CA2 |
| 7501098 | 28 | 7501098CD1 | 58 | 7501098CB1 | 4660380CA2 |
| 7503839 | 29 | 7503839CD1 | 59 | 7503839CB1 | |
| 7503698 | 30 | 7503698CD1 | 60 | 7503698CB1 | |

TABLE 2

| | | GenBank ID NO: | | |
|---|---|---|---|---|
| Polypeptide | Incyte | or PROTEOME | Probability | |
| SEQ ID NO: | Polypeptide ID | ID NO: | Score | Annotation |
| 1 | 1895273CD1 | g180279 | 5.40E−146 | [Homo sapiens] IgG Fc receptor I |
| | | | | van de Winkel, J. G. J. et al., (1991) |
| | | | | J. Biol. Chem. 266:13449-13455 |
| 6 | 6219465CD1 | g6649221 | 2.40E−184 | [Homo sapiens] selenoprotein N |
| | | | | Lescure, A. et al.,(1999) |
| | | | | J. Biol. Chem. 274:38147-38154 |
| 7 | 3576625CD1 | g4406679 | 1.10E−54 | [Homo sapiens] Human neuronal olfactomedin |
| | | | | related ER localized protein |
| | | | | Danielson, P. E. et al., (1994) |
| | | | | J. Neurosci. Res. 38:468-78 |
| 10 | 7714187CD1 | g3041877 | 1.30E−222 | [Homo sapiens] IB3089A |
| | | | | Nishiyama, H. et al. (1999) |
| | | | | Genes Chromosomes Cancer 26:171-5 |
| 11 | 5136540CD1 | g12656635 | 8.40E−54 | [Homo sapiens] (AF326351) transmembrane |
| | | | | gamma-carboxyglutamic acid |
| | | | | protein 4 TMG4 |
| | | | | Kulman, J. D. et al.,(2001) |
| | | | | Proc. Natl. Acad. Sci. U.S.A. 98:1370-1375 |
| 12 | 3277403CD1 | g9864185 | 8.10E−104 | [Drosophila melanogaster] Crossveinless 2 |
| | | | | Olson, D. J. et al., (2000) |
| | | | | Development 127:3947-3959 |
| 16 | 2768535CD1 | g7578787 | 8.30E−134 | AD021 protein [Homo sapiens] |
| 23 | 5685755CD1 | g307153 | 9.30E−27 | [Homo sapiens] Mac-2 binding protein |
| | | | | Koths, K., Taylor, E., Halenbeck, R., Casipit, C. |
| | | | | and Wang, A. (1993) Cloning and characterization |
| | | | | of a human Mac-2 binding protein, a new member of |
| | | | | the superfamily defined by the macrophage |
| | | | | scavenger receptor cysteine-rich domain |
| | | | | J. Biol. Chem. 268:14245-14249 |
| 26 | 2911343CD1 | g11225366 | 4.10E−13 | [Homo sapiens] testis specific leucine |
| | | | | rich repeat protein |
| 28 | 7501098CD1 | g12656635 | 2.6E−13 | [Homo sapiens] transmembrane |
| | | | | gamma-carboxyglutamic acid protein 4 TMG4 |
| | | | | Kulman, J. D. et al. (2001) Identification |
| | | | | of two novel transmembrane gamma- |
| | | | | carboxyglutamic acid proteins expressed |
| | | | | broadly in fetal and adult tissues. Proc. |
| | | | | Natl. Acad. Sci. U.S.A. 98:1370-1375 |
| | | 685373\|TMG4 | 2.2E−14 | [Homo sapiens][Plasma membrane] |
| | | | | Transmembrane gamma-carboxyglutamic |
| | | | | acid protein 4, a putative transmembrane |
| | | | | spanning protein which is a member of |
| | | | | the Gla family, contains one cytoplasmic |
| | | | | PPXY motif which may mediate interaction |
| | | | | with WW domain-containing proteins |
| | | | | Kulman, J. D. et al. (2001) Identification |
| | | | | of two novel transmembrane gamma - |
| | | | | carboxyglutamic acid proteins expressed |
| | | | | broadly in fetal and adult tissues. Proc. |
| | | | | Natl. Acad. Sci. U.S.A. 98:1370-1375. |

TABLE 3

| SEQ | Incyte | Amino | | |
|---|---|---|---|---|
| ID | Polypeptide | Acid | Signature Sequences, | Analytical Methods |
| NO: | ID | Residues | Domains and Motifs | and Databases |
| 1 | 1895273CD1 | 289 | Signal_cleavage: M1-G15 | SPSCAN |
| | | | Signal Peptide: M1-G15 | HMMER |
| | | | Immunoglobulin domain: G120-A177, G32-G85 | HMMER_PFAM |
| | | | Transmembrane domain: L4-L20, | TMAP |
| | | | P203-K230N-terminus is cytosolic | |
| | | | RECEPTOR FC IMMUNOGLOBULIN PD01270: | BLIMPS_PRODOM |
| | | | E33-F61, L131-R166, M1-S26 | |
| | | | GAMMA FC RECEPTOR I HIGH AFFINITY | BLAST_PRODOM |
| | | | IMMUNOGLOBULIN PRECURSOR FC GAMMA RI | |
| | | | PD013195: W238-T289 | |
| | | | RECEPTOR FC GAMMA AFFINITY IMMUNOGLOBULIN | BLAST_PRODOM |
| | | | PRECURSOR PROTEIN TRANSMEMBRANE | |
| | | | GLYCOPROTEIN SIGNAL PD002534: | |
| | | | W19-W64, L4-G15 | |
| | | | RECEPTOR FC GAMMA I HIGH AFFINITY | BLAST_PRODOM |
| | | | IMMUNOGLOBULIN PRECURSOR PROTEIN | |
| | | | TRANSMEMBRANE PD004578: S157-K237, | |
| | | | N65-V108 | |
| | | | GAMMA FC RECEPTOR I HIGH AFFINITY | BLAST_PRODOM |
| | | | IMMUNOGLOBULIN PRECURSOR FC GAMMA RI | |
| | | | PD013193: P107-T156 | |
| | | | IG-LIKE C2-TYPE DOMAIN | BLAST_DOMO |
| | | | DM03427P12314\|189-331: F104-G247, | |
| | | | P26151\|198-339: F104-S246, | |
| | | | I48471\|199-336: E102-K235 | |
| | | | MYELIN-ASSOCIATED GLYCOPROTEIN | BLAST_DOMO |
| | | | DM00682\|P12314\|1-187: G18-L103 | |
| | | | Potential Phosphorylation Sites: S25 S246 | MOTIFS |
| | | | S255 T30 T99 T150 T156 T164 T227 Y173 | |
| | | | Potential Glycosylation Sites: N67 | MOTIFS |
| | | | N74 N78 N110 N155 | |
| 2 | 70072222CD1 | 159 | Signal_cleavage: M1-A26 | SPSCAN |
| | | | Signal Peptide: M1-A26 | HMMER |
| | | | Transmembrane domain: P55-C83 | TMAP |
| | | | N-terminus is cytosolic | |
| | | | Potential Phosphorylation Sites: | MOTIFS |
| | | | S141 T90 T132 | |
| 3 | 3559223CD1 | 559 | Signal_cleavage: M1-S19 | SPSCAN |
| | | | Potential Phosphorylation Sites: S17 S31 | MOTIFS |
| | | | S66 S93 S187 S188 S242 S260 S267 S318 | |
| | | | S331 S412 S442 S446 S525 S549 T141 T148 | |
| | | | T160 T200 T245 T426 T467 T480 T488 | |
| | | | Potential Glycosylation Sites: N248 N386 | MOTIFS |
| 4 | 3441255CD1 | 1222 | Signal_cleavage: M1-F32 | SPSCAN |
| | | | Signal Peptide: M11-S30 | HMMER |
| | | | Signal Peptide: M11-E33 | HMMER |
| | | | Signal Peptide: M11-S35 | HMMER |
| | | | Transmembrane domain: P8-E34, P179-N196, | TMAP |
| | | | R773-S793, W801-L821N-Terminus is | |
| | | | non-cytosolic | |
| | | | Potential Phosphorylation Sites: S30 | MOTIFS |
| | | | S282 S444 S447 S465 S657 S701 S748 S806 | |
| | | | S849 S873 S909 S976 S1007 S1033 S1060 | |
| | | | T31 T54 T189 T249 T293 T416 T539 T672 | |
| | | | T842 T844 T903 T1020 T1148 T1210 | |
| | | | Potential Glycosylation Sites:N38 | MOTIFS |
| | | | N676 N698 N719 N884 | |
| 5 | 1958917CD1 | 696 | Signal_cleavage: M1-A27 | SPSCAN |
| | | | Signal Peptide: M3-A27 | HMMER |
| | | | Signal Peptide: M3-A34 | HMMER |
| | | | Transmembrane domain: S8-W36N-terminus | TMAP |
| | | | is non-cytosolic | |
| | | | do MUCIN; MUC5; TRACHEOBRONCHIAL; | BLAST_DOMO |
| | | | DM05454\|S55316\|1-317: Q355-S581 | |
| | | | Potential Phosphorylation Sites: S72 | MOTIFS |
| | | | S99 S122 S184 S383 S473 S475 S499 S506 | |
| | | | S511 S549 S612 S649 S662 T235 T248 | |
| | | | T512 T542 T618 | |
| | | | Potential Glycosylation Sites: N483 | MOTIFS |
| 6 | 6219465CD1 | 436 | Signal_cleavage: M1-A43 | SPSCAN |
| | | | Transmembrane domain: R25-A53, | TMAP |
| | | | V231-I258 N-terminus is non-cytosolic | |
| | | | EF-hand calcium-binding domain | BLIMPS_PRINTS |
| | | | BL00018: D80-F92 | |
| | | | Potential Phosphorylation Sites: | MOTIFS |
| | | | S77 S88 S107 S319 S332 S359 S385 | |
| | | | S404 T81 Y398 | |
| | | | Potential Glycosylation Sites: N156 | MOTIFS |
| 7 | 3576625CD1 | 652 | Signal_cleavage: M1-A27 | SPSCAN |
| | | | Signal Peptide: M1-A27 | HMMER |
| | | | Olfactomedin-like domain: G397-V652 | HMMER_PFAM |
| | | | Transmembrane domain: A4-S21N-termincus | TMAP |
| | | | is non-cytosolic | |
| | | | PROTEIN PRECURSOR SIGNAL MYOCILIN | BLAST_PRODOM |
| | | | TRABECULAR GLUCOCORTICOID | |
| | | | GLYCOPROTEIN OLFACTOMEDIN MESH-WORK | |
| | | | INDUCED RESPONSE PD006897: L440-T645 | |
| | | | Potential Phosphorylation Sites: S21 | MOTIFS |
| | | | S66 S82 S121 S155 S170 S242 S266 S314 | |
| | | | S345 S382 S439 S519 S551 S574 T25 T151 | |
| | | | T225 T275 T398 T565 T606 T624 Y75 Y236 | |
| | | | Potential Glycosylation Sites: N159 N183 | MOTIFS |
| 8 | 4765758CD1 | 91 | Signal_cleavage: M1-A42 | SPSCAN |
| | | | Signal Peptide: M20-A42 | HMMER |
| | | | Signal Peptide: M20-G44 | HMMER |
| | | | Citrate synthase signature: E6-D67 | PROFILESCAN |
| | | | Ribosomal protein S24e signature: K3-A57 | PROFILESCAN |
| | | | Potential Phosphorylation Sites: | MOTIFS |
| | | | S77 S82 T22 T70 T73 | |
| 9 | 7236661CD1 | 155 | Signal_cleavage: M1-G17 | SPSCAN |
| | | | Signal Peptide: M1-G17 | HMMER |
| | | | Signal Peptide: M1-A20 | HMMER |
| | | | Potential Phosphorylation Sites: S38 | MOTIFS |
| | | | S43 S91 S95 S135 T47 T90 T115 | |
| 10 | 7714187CD1 | 765 | Signal_cleavage: M1-A33 | SPSCAN |
| | | | Signal Peptide: M15-A33 | HMMER |
| | | | Signal Peptide: M15-A34 | HMMER |
| | | | Signal Peptide: M15-D37 | HMMER |
| | | | Signal Peptide: M15-Q38 | HMMER |
| | | | Signal Peptide: M15-A40 | HMMER |
| | | | Signal Peptide: M15-A33 | HMMER |
| | | | Transmembrane domain: G8-A33 | TMAP |
| | | | Membrane attack complex | PROFILESCAN |
| | | | components/perforin signature: L103-K155 | |
| | | | IB3089A PD147692: M1-C765 | BLAST_PRODOM |
| | | | Potential Phosphorylation Sites: S42 | MOTIFS |
| | | | S49 S72 S157 S218 S267 S315 S361 | |
| | | | S380 S418 S523 S528 S539 S563 S579 | |
| | | | S592 S735 T119 T191 T309 T442 T504 | |
| | | | T536 T599 T620 T761 | |
| | | | Potential Glycosylation Sites: N168 | MOTIFS |
| | | | N337 N456 N562 N609 N641 | |
| 11 | 5136540CD1 | 150 | Signal_cleavage: M1-G17 | SPSCAN |
| | | | Signal Peptide: M1-G17 | HMMER |
| | | | Signal Peptide: M1-P19 | HMMER |
| | | | Vitamin K-dependent carboxylation/gamma- | HMMER_PFAM |
| | | | carboxyglutamic (GLA) domain: L57-A98 | |
| | | | Vitamin K-dependent carboxylation | PROFILESCAN |
| | | | domain: V35-S110 | |
| | | | Coagulation factor GLA domain signature | BLIMPS_PRINTS |
| | | | PR00001: V84-A98, D56-C69, N70-F83 | |
| | | | GLA DOMAIN DM00454\|P08709\|22-100: K29-W93 | BLAST_DOMO |
| | | | GLA DOMAIN DM00454\|P00742\|2-80: G32-W93 | BLAST_DOMO |
| | | | GLA DOMAIN DM00454\|P25155\|2-80: C21-W93 | BLAST_DOMO |
| | | | GLA DOMAIN DM00454\|S49075\|2-80: G32-W93 | BLAST_DOMO |
| | | | Vitamin K-dependent carboxylation domain: D56-W93 | MOTIFS |
| | | | Potential Phosphorylation Sites: S38 S97 | MOTIFS |
| | | | S110 S120 T37 T102 | |
| 12 | 3277403CD1 | 685 | Signal_cleavage: M1-G33 | SPSCAN |
| | | | Signal Peptide: R16-A39 | HMMER |
| | | | Signal Peptide: L10-A39 | HMMER |
| | | | Trypsin Inhibitor like cysteine rich | HMMER_PFAM |
| | | | domain: C629-C682 | |
| | | | von Willebrand factor type C domain: | HMMER_PFAM |
| | | | C108-C163, C166-C224, C301-C357, | |
| | | | C238-C289, C50-C105 | |
| | | | von Willebrand factor type D domain: | HMMER_PFAM |
| | | | C364-N514 | |
| | | | C-terminal cystine knot BL01185: | BLIMPS_BLOCKS |
| | | | C238-C286, K351-V389 | |
| | | | SIGNAL PRECURSOR GLYCOPROTEIN | BLAST_PRODOM |
| | | | VITELLOGENIN CELL REPEAT WILLEBRAND VON | |
| | | | ALPHA TECTORIN PD001080: C331-C663 | |
| | | | do MUCIN; VON; WILLEBRAND; HEMOCYTIN; | BLAST_DOMO |
| | | | DM01378\|P04275\|512-692: A486-L669 | |
| | | | do MUCIN; VON; WILLEBRAND; HEMOCYTIN; | BLAST_DOMO |
| | | | DM01378\|P04275\|160-333: G495-P666 | |
| | | | do MUCIN; VON; WILLEBRAND; HEMOCYTIN; | BLAST_DOMO |
| | | | DM01378\|P04275\|986-1176: K492-H664 | |
| | | | do MUCIN; VON; WILLEBRAND; HEMOCYTIN; | BLAST_DOMO |
| | | | DM01378\|P98092\|370-549: K490-C663 | |
| | | | Vitamin K-dependent carboxylation | MOTIFS |
| | | | domain: K271-F308 | |
| | | | VWFC domain signature: C67-C105, | MOTIFS |
| | | | C186-C224, C319-C357 | |
| | | | Potential Phosphorylation Sites: | MOTIFS |
| | | | S118 S123 S273 S479 S525 S573 T78 T260 | |
| | | | T358 T437 T466 T549 | |
| | | | Potential Glycosylation Sites: N30 N116 | MOTIFS |
| | | | N247 N255 N318 N441 | |
| 13 | 1517569CD1 | 126 | Signal_cleavage: M1-G33 | SPSCAN |
| | | | Transmembrane Region: L58-S86 | TMAP |
| | | | Potential Phosphorylation Sites: | MOTIFS |
| | | | S56 T13 T27 T52 Y71 | |
| | | | Potential Glycosylation Sites: N11 | MOTIFS |
| 14 | 2415991CD1 | 149 | Signal Peptide: M1-G21 | HMMER |
| | | | Potential Phosphorylation Sites: S122 | MOTIFS |
| | | | Potential Glycosylation Sites: N33 | MOTIFS |
| 15 | 2735742CD1 | 114 | Signal_cleavage: M1-A18 | SPSCAN |
| | | | S63 S72 S105 | MOTIFS |
| 16 | 2768535CD1 | 519 | Signal_cleavage: M1-L60 | SPSCAN |
| | | | Transmembrane Region: L38-G56 | TMAP |
| | | | N-terminus is cytosolic | |
| | | | Potential Phosphorylation Sites: S28 S29 | MOTIFS |
| | | | S80 S113 S199 S217 S239 S296 S327 T2 | |
| | | | T34 T110 T124 | |
| | | | Potential Glycosylation Sites: | MOTIFS |
| | | | N98 N289 N322 | |
| 17 | 6848851CD1 | 1164 | Signal_cleavage: M1-A45 | SPSCAN |
| | | | Transmembrane Region: S901-A923 | TMAP |
| | | | N-terminus is cytosolic | |
| | | | Potential Phosphorylation Sites: | MOTIFS |
| | | | S18 S19 S65 S79 S107 S108 S110 S116 | |
| | | | S128 S132 S216 S240 S241 S358 S379 | |
| | | | S486 S506 S628 S665 S725 S956 S1027 | |
| | | | T71 T78 T98 T112 T114 T162 T165 T209 T211 | |
| | | | T225 T600 T604 T843 T1017 T1062 T1145 Y859 | |
| | | | Potential Glycosylation Sites: | MOTIFS |
| | | | N377 N413 N935 N1009 | |
| 18 | 7040722CD1 | 112 | Signal_cleavage: M1-E19 | SPSCAN |
| | | | Signal Peptide: M2-G23 | HMMER |
| | | | Potential Phosphorylation Sites: | MOTIFS |
| | | | S102 T86 | |
| | | | Potential Glycosylation Sites: N103 | MOTIFS |
| 19 | 6430290CD1 | 170 | Signal_cleavage: M1-G54 | SPSCAN |
| | | | Transmembrane Region: C23-S51 | TMAP |
| | | | N-terminus is non-cytosolic | |
| | | | Potential Phosphorylation Sites: | MOTIFS |
| | | | S9 S51 S143 S144 T66 T121 | |
| 20 | 2640251CD1 | 80 | Signal_cleavage: M1-A42, M18-A42, M14-A42 | SPSCAN |
| | | | Signal Peptide: M18-S44 | HMMER |
| | | | Signal Peptide: M18-A42 | HMMER |
| | | | Potential Phosphorylation Sites: S20 T4 T53 | MOTIFS |
| 21 | 3839350CD1 | 118 | Signal_cleavage: M1-A18 | SPSCAN |
| | | | Signal Peptide: M1-A18 | HMMER |
| | | | Potential Phosphorylation Sites: S20 S50 T6 | MOTIFS |
| 22 | 6393813CD1 | 140 | Signal_cleavage: M1-A42, M16-A42 | SPSCAN |
| | | | Potential Phosphorylation Sites: | MOTIFS |
| | | | S23 S33 S61 S72 S122 S124 | |
| 23 | 5685755CD1 | 478 | Signal_cleavage: M1-R31 | SPSCAN |
| | | | Signal Peptide: M1-A32 | HMMER |
| | | | BTB/POZ domain: Q47-L162 | HMMER_PFAM |
| | | | Transmembrane domain: G10-T26N-terminus | TMAP |
| | | | is non-cytosolic | |
| | | | PROTEIN C ASSOCIATED MAMA CYCLOPHILIN | BLAST_PRODOM |
| | | | PEPTIDYLPROLYL ISOMERASE PANCREAS | |
| | | | CANCER ASSOCIATED MAC2 BINDING | |
| | | | PD014408: L53-A323, S338-P362 | |
| | | | SPERACT RECEPTOR AMINO-TERMINAL | BLAST_DOMO |
| | | | DM05438\|A47161\|126-585: S42-P345 | |
| | | | Potential Phosphorylation Sites: | MOTIFS |
| | | | S346 S361 S368 S400 S434 S452 T179 | |
| | | | T355 T476 | |
| | | | Potential Glycosylation Sites:N44 | MOTIFS |
| | | | N61 N100 N195 N307 | |
| 24 | 71728459CD1 | 80 | Signal_cleavage: M1-A15 | SPSCAN |
| | | | Signal Peptide: M1-A15 | HMMER |
| | | | Signal Peptide: M1-G28 | HMMER |
| | | | Transmembrane domain: T4-W23 | TMAP |
| | | | Potential Phosphorylation Sites: | MOTIFS |
| | | | S26 S35 S54 S68 | |
| 25 | 1904303CD1 | 505 | Signal_cleavage: M1-A20 | SPSCAN |
| | | | Signal Peptide: M1-A20 | HMMER |
| | | | Signal Peptide: M1-G24 | HMMER |
| | | | Transmembrane domain: V312-L340, | TMAP |
| | | | P467-L494N-terminus is non-cytosolic | |
| | | | Potential Phosphorylation Sites: | MOTIFS |
| | | | S21 S174 S237 S364 S368 S413 S426 | |
| | | | T46 T176 T203 T248 T288 T338 T411 | |
| | | | Potential Glycosylation Sites: | MOTIFS |
| | | | N220 N229 N278 N336 | |
| 26 | 2911343CD1 | 321 | Signal_cleavage: M39-A96 | SPSCAN |
| | | | Leucine Rich Repeat: N67-P88, S89-P110, | HMMER_PFAM |
| | | | C111-P131, S133-L162, E45-Q66 | |
| | | | Potential Phosphorylation Sites: S36 S89 | MOTIFS |
| | | | S175 S182 S183 S196 S304 S319 T23 | |
| | | | T28 T42 T48 T58 T124 T145 T232 T260 | |
| | | | T274 T311 T315 | |
| | | | Potential Glycosylation Sites: N26 N138 | MOTIFS |
| 27 | 7500308CD1 | 60 | Signal_cleavage: M1-P21 | SPSCAN |
| | | | Signal Peptide: MI-125, M1-L26 | HMMER |
| | | | Potential Phosphorylation Sites: S42 T33 | MOTIFS |
| 28 | 7501098CD1 | 45 | Signal_cleavage: M1-G17 | SPSCAN |
| | | | Signal Peptide: M1-G17 | HMMER |
| | | | Signal Peptide: M1-G24 | HMMER |
| | | | Signal Peptide: M1-P19 | HMMER |
| | | | Histidine acid phosphatases | PROFILESCAN |
| | | | signatures: M1-A44 | |
| 29 | 7503839CD1 | 43 | Signal_cleavage: M1-A13 | SPSCAN |
| | | | Signal Peptide: M1-S18 | HMMER |
| | | | Potential Phosphorylation Sites: | MOTIFS |
| | | | S18 S19 T40 | |
| 30 | 7503698CD1 | 456 | Signal_cleavage: M1-A20 | SPSCAN |
| | | | Signal Peptide: M1-A19, M1-S21, | HMMER |
| | | | M1-A20, M1-G24 | |
| | | | Potential Phosphorylation Sites: | MOTIFS |
| | | | S21 S174 S315 S319 S364 S377 T46 | |
| | | | T176 T203 T239 T289 T362 | |
| | | | Potential Glycosylation Sites: | MOTIFS |
| | | | N220 N229 N287 | |

TABLE 4

| Polynucleotide | |
|---|---|
| SEQ ID NO:/ | |
| Incyte ID/Sequence | |
| Length | Sequence Fragments |
| 31/1895273CB1/ | 1-211, 1-320, 67-288, 67-335, 67-644, 69-216, 69-230, 69-286, 69-293, 69-331, 69-344, 69-358, 69-363, 69-481, 69- |
| 1053 | 495, 69-591, 69-607, 69-617, 69-636, 112-506, 132-985, 150-394, 186-786, 188-967, 190-641, 190-726, 190-727, |
| | 241-713, 243-711, 258-1000, 264-950, 268-877, 276-991, 286-712, 287-995, 310-953, 326-614, 367-1027, 423-606, |
| | 428-1036, 429-679, 434-724, 455-709, 459-653, 459-676, 459-998, 471-1003, 483-697, 488-1032, 489-971, 503- |
| | 1050, 507-1023, 508-1053, 598-645, 743-1051 |
| 32/70072222CB1/ | 1-550, 1-613, 4-295, 28-284, 360-627, 362-623, 372-623, 377-664, 406-930, 430-830, 451-607, 459-1027, 459- |
| 1579 | 1048, 544-1022, 564-1027, 590-1146, 609-1134, 665-1320, 676-1263, 691-1053, 694-1146, 709-1320, 730-1209, |
| | 740-1263, 746-1061, 751-1015, 751-1223, 772-1205, 784-1270, 792-1332, 805-1234, 805-1246, 815-1322, 816- |
| | 1460, 843-1362, 860-1263, 876-1362, 904-1160, 925-1492, 943-1575, 976-1544, 1024-1307, 1033-1579, 1041- |
| | 1069, 1041-1339, 1041-1341, 1041-1342, 1041-1343, 1041-1346, 1041-1347, 1045-1172, 1046-1069, 1054-1579, |
| | 1057-1347, 1084-1544, 1104-1579, 1120-1579 |
| 33/3559223CB1/ | 1-471, 51-284, 128-825, 129-705, 222-334, 253-785, 282-593, 283-699, 283-786, 357-400, 408-1003, 457-699, 457- |
| 2440 | 826, 467-1050, 469-993, 520-1132, 534-752, 585-866, 585-1061, 635-1291, 703-966, 747-1291, 781-1350, 813- |
| | 1417, 820-1034, 906-1151, 925-1291, 942-1341, 1021-1294, 1064-1326, 1083-1591, 1110-1380, 1110-1673, 1132- |
| | 1482, 1164-1340, 1169-1705, 1237-1490, 1237-1553, 1237-1607, 1246-1793, 1253-1758, 1253-1843, 1270-1876, |
| | 1348-1997, 1418-1729, 1474-2026, 1486-1705, 1495-1683, 1495-1716, 1517-2020, 1551-2206, 1608-1890, 1611- |
| | 1754, 1653-2268, 1665-2002, 1671-1947, 1681-1942, 1691-1801, 1731-2269, 1741-2278, 1752-1821, 1759-2418, |
| | 1780-2270, 1793-2408, 1819-2366, 1827-2321, 1829-2018, 1829-2358, 1839-2404, 1846-2362, 1883-2345, 1889- |
| | 2337, 1901-2118, 1908-2061, 1908-2365, 1913-2171, 1913-2435, 1935-2420, 1936-2174, 1943-2314, 1957-2423, |
| | 1963-2440, 1968-2424, 1969-2423, 1970-2422, 1971-2411, 1975-2423, 1986-2423, 1997-2440, 2000-2266, 2009- |
| | 2420, 2018-2440, 2019-2277, 2020-2414, 2023-2440, 2036-2432, 2038-2440, 2043-2424, 2044-2434, 2053-2424, |
| | 2066-2420, 2070-2420, 2071-2420, 2095-2395, 2141-2440, 2146-2420, 2147-2420, 2157-2416, 2157-2430, 2158- |
| | 2334, 2158-2376, 2158-2390, 2201-2418, 2279-2420, 2311-2420 |
| 34/3441255CB1/ | 1-349, 1-651, 243-430, 489-4133, 655-1312, 665-1263, 925-1207, 925-1545, 932-1543, 945-1617, 1040-1585, 1260- |
| 4133 | 1709, 1293-1597, 1388-1675, 1388-2004, 1681-1927, 1700-1936, 1700-2164, 1725-2284, 1796-2295, 1809-2215, |
| | 2074-2304, 2074-2336, 2074-2465, 2127-2774, 2148-2409, 2205-2442, 2515-2735, 2515-2918, 2515-3045, 2517- |
| | 2784, 2640-2908, 2659-3187, 2725-3340, 2743-2990, 2776-3233, 2777-3023, 2790-3014, 2794-3321, 2832-3337, |
| | 2832-3649, 2841-3160, 2872-3447, 2948-3199, 2988-3368, 3027-3368, 3105-3321, 3237-3507, 3327-3983, 3327- |
| | 4004, 3381-4029, 3406-3904, 3427-4003, 3514-4077, 3594-4128, 3632-4077, 3653-3886, 3709-4018 |
| 35/1958917CB1/ | 1-70, 1-623, 2-516, 185-463, 356-989, 464-660, 464-846, 609-803, 609-896, 609-1018, 609-1133, 609-1137, 617- |
| 4689 | 1058, 617-1201, 660-1445, 676-1349, 677-904, 679-1420, 687-1445, 706-935, 709-1445, 725-1137, 726-1443, 779- |
| | 1137, 782-904, 852-1137, 936-1137, 977-1137, 1000-1137, 1029-1202, 1044-1137; 1089-1137, 1097-1137, 1113- |
| | 1137, 1138-1327, 1203-1327, 1203-1502, 1213-1744, 1328-1502, 1328-1616, 1375-1766, 1394-1654, 1394-1806, |
| | 1394-1827, 1489-2117, 1503-1744, 1617-1744, 1617-2054, 1655-2312, 1745-2054- 1777-2316, 1848-2366, 2001- |
| | 2435, 2047-2477, 2055-2275, 2092-2575, 2132-2409, 2180-2748, 2273-2762, 2283-2724, 2313-2555, 2355-2802, |
| | 2369-2917, 2467-3119, 2530-2979, 2547-3208, 2572-3124, 2576-2805, 2591-2896, 2603-2875, 2670-3140, 2671- |
| | 2971, 2683-3224, 2683-3269, 2695-3261, 2711-3335, 2821-3340, 2821-3354, 3048-3544, 3122-3694, 3160-3603, |
| | 3172-3376, 3217-3410, 3217-3752, 3245-3782, 3332-3875, 3349-3924, 3435-3924, 3503-4186, 3503-4244, 3504- |
| | 3748, 3551-3840, 3551-4036, 3611-4005, 3631-3845, 3714-3745, 3714-3750, 3714-3751, 3714-3753, 3714-3780, |
| | 3714-3781, 3714-3792, 3714-3797, 3714-3798, 3714-3801, 3714-3828, 3714-3829, 3714-3846, 3714-3848, 3714- |
| | 3876, 3714-3896, 3717-3755, 3718-3753, 3718-3800, 3719-4008, 3721-4145, 3735-3893, 3735-3896, 3761-3795, |
| | 3761-3801, 3761-3830, 3761-3887, 3761-3892, 3761-3923, 3761-3944, 3766-3944, 3768-3944, 3777-3850, 3782- |
| | 4032, 3782-4301, 3809-3843, 3809-3848, 3809-3877, 3809-3935, 3809-3940, 3809-3943, 3813-3847, 3813-3895, |
| | 3814-3943, 3816-3943, 3830-3944, 3856-3890, 3856-3896, 3856-3925, 3856-3944, 3856-3945, 3860-3894, 3860- |
| | 4689, 3861-3944, 3863-3944, 3877-3943, 3904-3938, 3904-3943, 3904-3949, 3904-4188, 3908-3942, 3909-3943, |
| | 3911-3943, 3930-4402, 3976-4498, 3979-4543, 3982-4587, 3993-4262, 3993-4480, 4083-4304, 4108-4359, 4136- |
| | 4328, 4136-4416, 4246-4537, 4360-4645, 4491-4547, 4628-4651 |
| 36/6219465CB1/ | 1-1311, 168-355, 168-483, 168-548, 168-573, 168-588, 168-592, 168-601, 168-643, 168-646, 168-679, 168-682, 168- |
| 3294 | 686, 168-689, 168-696, 168-715, 168-745, 168-765, 168-797, 168-819, 168-822, 168-835, 168-843, 169-745, 171- |
| | 551, 172-577, 172-587, 172-608, 172-745, 174-745, 175-771, 178-678, 178-733, 178-745, 187-642, 188-526, 189- |
| | 526, 202-498, 211-798, 216-745, 224-349, 224-532, 224-535, 224-745, 226-721, 229-745, 230-452, 230-525, 237- |
| | 415, 237-596, 238-526, 251-436, 263-772, 278-813, 306-951, 325-967, 326-800, 338-510, 341-510, 341-868, 347- |
| | 745, 382-790, 397-970, 513-745, 514-977, 561-1309, 567-800, 568-804, 570-772, 570-843, 593-1149, 641-1123, |
| | 641-1266, 663-1116, 698-1255, 703-1188, 727-1173, 740-1313, 755-1176, 763-1333, 765-1384, 772-1298, 785- |
| | 1417, 788-1148, 788-1366, 788-1381, 788-1389, 788-1467, 820-1413, 829-1319, 835-1313, 836-1437, 837-1511, |
| | 854-1422, 870-1516, 873-1212, 882-1101, 918-986, 936-986, 940-986, 951-986, 962-986, 1014-1226, 1014-1443, |
| | 1014-1456, 1014-1485, 1014-1544, 1014-1548, 1014-1685, 1015-1476, 1016-1105, 1031-1561, 1031-1588, |
| | 1046-1528, 1049-1458, 1049-1694, 1079-1590, 1086-1604, 1089-1469, 1096-1288, 1108-1730, 1109-1626, 1110- |
| | 1532, 1113-1384, 1113-1564, 1129-1341, 1136-1783, 1142-1640, 1144-1760, 1172-1692, 1177-1767, 1183-1790, |
| | 1186-1773, 1190-1524, 1194-1743, 1194-1790, 1200-1642, 1202-1790, 1207-1790, 1211-1783, 1215-1712, 1215- |
| | 1805, 1217-1747, 1223-1776, 1224-1790, 1236-1790, 1236-1864, 1237-1827, 1244-1682, 1244-1790, 1257-1905, |
| | 1259-1835, 1289-1790, 1292-1873, 1294-1648, 1295-1541, 1302-1790, 1335-1773, 1337-1790, 1344-1978, 1352- |
| | 1790, 1358-1663, 1370-2054, 1378-1888, 1411-1922, 1434-1596, 1434-1927, 1439-2074, 1445-1868, 1461-1868, |
| | 1477-1790, 1505-1958, 1506-1913, 1516-1807, 1529-1790, 1549-1793, 1587-1818, 1587-1889, 1587-1998, 1587- |
| | 2083, 1587-2139, 1587-2224, 1587-2254, 1589-2051, 1595-1840, 1640-2133, 1644-1960, 1646-2251, 1647-2275, |
| | 1648-2280, 1667-1970, 1670-1995, 1671-2250, 1672-2225, 1673-1939, 1677-2245, 1715-2331, 1720-1986, 1730- |
| | 2238, 1736-2233, 1753-1928, 1766-2356, 1770-2293, 1777-2448, 1792-2284, 1792:-2380, 1793-2244, 1797-2050, |
| | 1799-2441, 1804-2342, 1818-2225, 1822-2434, 1829-2135, 1846-2088, 1862-2540, 1885-2470, 1890-2483, 1892- |
| | 2145, 1909-2535, 1912-2526, 1933-2130, 1943-2516, 1954-2582, 1963-2492, 1971-2559, 1971-2614, 1980-2524, |
| | 1983-2503, 1989-2438, 1993-2464, 1999-2547, 1999-2733, 2008-2492, 2009-2420, 2009-2535, 2010-2614, 2017- |
| | 2464, 2051-2332, 2065-2669, 2071-2535, 2104-2689, 2109-2649, 2117-2669, 2118-2379, 2125-2748, 2131-2558, |
| | 2136-2639, 2146-2739, 2146-2802, 2155-2686, 2159-2821, 2161-2685, 2162-2748, 2170-2692, 2189-2702, 2200- |
| | 2740, 2222-2723, 2250-2764, 2264-2780, 2293-2720, 2296-2537, 2299-2748, 2328-2575, 2359-2608, 2359-2929, |
| | 2363-2599, 2381-2679, 2385-2729, 2393-2693, 2476-2690, 2493-3030, 2494-2712, 2494-2721, 2530-2776, 2608- |
| | 2872, 2618-2850, 2667-2925, 2667-2948, 2698-2949, 2700-2947, 2701-2920, 2706-2989, 2763-2964, 2763-2970, |
| | 2776-3020, 2781-2976, 2793-3038, 2859-3020, 2869-3140, 2967-3239, 3040-3186, 3040-3294 |
| 37/3576625CB1/ | 1-90, 1-588, 62-157, 62-354, 62-464, 62-551, 62-564, 62-582, 62-686, 67-282, 67-571, 68-623, 71-657, 75-618, 78- |
| 3635 | 571, 78-582, 80-493, 91-1959, 98-332, 98-354, 98-543, 98-549, 108-677, 125-756, 130-815, 175-814, 227-722, 245- |
| | 892, 258-622, 337-1004, 348-416, 355-433, 396-779, 423-661, 461-613, 677-1098, 792-1075, 798-1386, 810-1458, |
| | 818-1106, 866-1113, 876-1458, 935-1098, 1133-1568, 1141-1713, 1153-1445, 1167-1598, 1169-1383, 1355-1735, |
| | 1355-1739, 1355-1745, 1355-1794, 1355-1812, 1355-1836, 1355-1852, 1355-1856, 1355-1885, 1355-1887, 1355- |
| | 1891, 1355-1918, 1355-1926, 1355-1962, 1355-1976, 1356-1891, 1389-1695, 1389-1900, 1389-1965, 1389-2010, |
| | 1389-2017, 1414-1891, 1556-2122, 1556-2177, 1556-2185, 1556-2202, 1556-2216, 1567-1937, 1575-1988, 1578- |
| | 2212, 1579-2035, 1579-2081, 1579-2118, 1579-2215, 1579-2216, 1580-2190, 1581-2212, 1584-2216, 1588-1891, |
| | 1588-2216, 1641-2212, 1654-2216, 1658-1963, 1660-2216, 1674-2243, 1680-2212, 1714-2230, 1719-2295, 1726- |
| | 2121, 1734-1869, 1748-2305, 1752-2334, 1753-2203, 1760-2419, 1783-2305, 1806-2367, 1817-2393, 1866-2332, |
| | 1868-2527, 1877-2525, 1883-2554, 1884-2071, 1910-2141, 1919-2565, 1930-2345, 1934-2595, 1952-2422, 1996- |
| | 2712, 2040-2525, 2044-2726, 2053-2630, 2058-2754, 2092-2616, 2107-2707, 2114-2736, 2127-2713, 2128-2659, |
| | 2159-2740, 2168-2510, 2231-2749, 2240-2707, 2245-2491, 2246-2885, 2285-2961, 2320-2972, 2330-2895, 2350- |
| | 2806, 2380-2903, 2393-3096, 2495-2705, 2496-3107, 2511-3203, 2547-2927, 2553-3249, 2573-3263, 2602-3263, |
| | 2607-3232, 2623-3100, 2626-2882, 2643-3294, 2688-2943, 2689-3407, 2699-3154, 2699-3249, 2712-3274, 2719- |
| | 3350, 2734-3203, 2752-2993, 2759-3413, 2770-3455, 2773-3326, 2773-3437, 2779-3440, 2783-3146, 2796-2825, |
| | 2856-3402, 2863-3154, 2869-3322, 2887-3435, 2932-3635, 2947-3523, 2951-3473, 3002-3497, 3127-3635, 3129- |
| | 3522, 3131-3352, 3195-3431, 3200-3460, 3216-3483 |
| 38/4765758CB1/ | 1-280, 1-611, 23-635 |
| 635 | |
| 39/7236661CB1/ | 1-56, 1-210, 1-379, 1-414, 1-503, 1-565, 1-793, 43-679, 63-679, 130-565, 202-793 |
| 793 | |
| 40/7714187CB1/ | 1-140, 15-532, 15-874, 158-262, 189-784, 379-662, 485-1033, 680-1171, 986-1068, 1062-1612, 1069-1599, 1292- |
| 2741 | 1588, 1373-1629, 1374-1843, 1404-2741, 1585-1828, 1585-2271, 1587-1822, 1682-2250, 2065-2734 |
| 41/5136540CB1/ | 1-252, 137-209, 137-412, 137-556, 137-575, 137-583, 137-597, 137-707, 137-738, 137-767, 145-191, 169-699,290- |
| 1074 | 675, 298-791, 315-574, 417-901, 426-901, 428-1071, 428-1074, 431-518, 452-1026, 468-1062, 488-1048, 518-771, |
| | 518-795, 518-920, 518-1062, 521-1054, 523-773, 523-1068, 527-974, 536-980, 561-785, 574-1068, 595-1070, 595- |
| | 1071, 626-1071, 637-1071, 647-1058, 811-1071, 984-1068 |
| 42/3277403CB1/ | 1-76, 1-632, 7-76, 19-71, 21-76, 119-426, 189-458, 234-750, 269-526, 381-2519, 472-1015, 667-1197, 994-1487, |
| 2560 | 1224-1446, 1281-1385, 1453-1603, 1516-1790, 1516-1967, 1516-2010, 1620-2113, 1621-2213, 1677-1962, 1677- |
| | 2201, 1711-1976, 1711-1999, 1743-2271, 1858-2560, 1863-2066, 1865-2303, 2112-2358, 2528-2559 |
| 43/1517569CB1/ | 1-541, 1-604, 12-686, 47-797, 124-507, 179-765, 198-661, 234-822, 237-811, 275-947, 377-1021, 414-1020, 430- |
| 5833 | 909, 472-1022, 517-986, 547-1067, 640-1021, 713-1086, 790-1268, 793-1366, 793-1446, 797-1333, 797-1443, 797- |
| | 1453, 799-1225, 799-1333, 850-1493, 949-1544, 1064-1706, 1073-1570, 1122-1724, 1131-1704, 1137-1707, 1218- |
| | 1916, 1279-1535, 1392-1617, 1501-1559, 1505-1755, 1505-1971, 1522-1749, 1609-1871, 1609-1877, 1609-2052, |
| | 1824-2236, 1930-2502, 1963-2559, 1980-2641, 1981-2454, 2019-2590, 2038-2544, 2038-2672, 2038-2706, 2149- |
| | 2767, 2175-2494, 2200-2428, 2200-2692, 2201-2738, 2211-2858, 2220-2931, 2265-2968, 2267-2463, 2268-2480, |
| | 2296-2888, 2333-2588, 2352-2971, 2355-2614, 2478-3083, 2479-3121, 2487-3119, 2493-3096, 2523-3136, 2552- |
| | 3021, 2589-3194, 2591-3249, 2598-3150, 2608-3176, 2619-3303, 2627-3219, 2635;3357, 2638-3040, 2639-3049, |
| | 2639-3211, 2640-3052, 2643-3321, 2648-3147, 2677-3184, 2702-3275, 2712-2972, 2715-2986, 2715-3188, 2720- |
| | 3099, 2733-3428, 2789-3021, 2872-3480, 2882-3443, 2882-3571, 2884-3350, 2888-3388, 2888-3591, 2889-3167, |
| | 2890-3360, 2948-3681, 2954-3443, 2960-3664, 2975-3571, 2976-3689, 2986-3652; 2991-3692, 2997-3685, 3026- |
| | 3579, 3032-3667, 3048-3713, 3064-3530, 3080-3375, 3085-3729, 3109-3362, 3114-3548, 3118-3722, 3118-3726, |
| | 3119-3399, 3119-3722, 3120-3693, 3135-3738, 3136-3736, 3138-3736, 3143-3377, 3148-3739, 3153-3415, 3160- |
| | 3435, 3160-3670, 3160-3734, 3162-3713, 3162-3738, 3164-3422, 3173-3424, 3179-3688, 3183-3517, 3187-3739, |
| | 3188-3738, 3193-3728, 3206-3482, 3207-3741, 3209-3692, 3213-3420, 3231-3525, 3231-3725, 3231-3743, 3232- |
| | 3738, 3234-3738, 3243-3508, 3252-3741, 3292-3858, 3299-3858, 3699-4031, 3699-4088, 3718-4036, 3749-3968, |
| | 3749-4255, 3749-4285, 3814-4000, 3926-4281, 4087-4111, 4087-4115, 4087-4165, 4087-4196, 4087-4200, 4087- |
| | 4250, 4089-4170, 4094-4250, 4126-4250, 4128-4717, 4137-4250, 4141-4165, 4172-4316, 4172-4333, 4172-4335, |
| | 4179-4431, 4211-4385, 4211-4394, 4213-4335, 4222-4491, 4226-4250, 4257-4335, 4264-4335, 4296-4335, 4298- |
| | 4335, 4307-4335, 4311-4536, 4408-4674, 4408-4963, 4487-4797, 4664-4956, 4671-5297, 4871-5193, 4871-5396, |
| | 4957-5255, 5024-5292, 5024-5586, 5033-5297, 5085-5238, 5098-5365, 5158-5369, 5391-5630, 5406-5684, 5406- |
| | 5699, 5406-5700, 5406-5714, 5408-5691, 5442-5718, 5513-5740, 5542-5819, 5549-5698, 5584-5833, 5607-5833, |
| | 5665-5727 |
| 44/2415991CB1/ | 1-707, 230-774, 245-538, 308-596, 351-1022, 419-681, 419-1022, 451-911, 451-959, 487-707, 487-727, 487-989, |
| 2264 | 598-906, 599-933, 611-858, 620-833, 661-1022, 713-1308, 786-1423, 798-1084, 875-1107, 890-1681, 892-1474, |
| | 903-1172, 927-1197, 927-1227, 947-1173, 1009-1271, 1022-1291, 1171-1756, 1174-1435, 1175-1471, 1206-1423, |
| | 1244-1760, 1274-1463, 1298-1559, 1308-1539, 1308-1722, 1308-1884, 1310-1590, 1311-1521, 1321-1591, 1344- |
| | 1623, 1344-1997, 1359-1565, 1395-1654, 1453-1744, 1510-2164, 1528-2178, 1536-1819, 1539-1827, 1570-2221, |
| | 1592-2216, 1644-1875, 1644-2155, 1648-2225, 1698-1959, 1730-1959, 1747-2264, 1748-1914, 1757-2019, 1812- |
| | 2061, 1817-2043, 1893-2108, 1895-2166, 2037-2248, 2054-2264, 2063-2134 |
| 45/2735742CB1/ | 1-625, 326-449, 326-502, 326-532, 326-533, 326-534, 326-544, 326-548, 326-549, 326-552, 326-557, 326-561, 326- |
| 1638 | 568, 326-569, 326-571, 326-572, 326-573, 326-574, 326-575, 326-578, 326-579, 325-584, 326-587, 326-590, 326- |
| | 599, 326-601, 326-610, 326-662, 326-747, 326-771, 326-836, 326-848, 326-894, 345-566, 362-586, 362-595, 372- |
| | 624, 418-618, 460-719, 482-725, 516-675, 582-847, 588-715, 599-885, 601-803, 601-814, 627-986, 633-884, 646- |
| | 900, 653-972, 680-895, 680-904, 684-917, 711-1000, 738-945, 757-993, 763-1019, 764-995, 798-1042, 805-1053, |
| | 830-1101, 878-1102, 889-1286, 914-1526, 931-1157, 937-1152, 942-1201, 942-1519, 947-1156, 947-1321, 958- |
| | 1183, 965-1188, 965-1195, 965-1514, 997-1566, 1019-1292, 1033-1269, 1050-1262, 1085-1319, 1112-1389, 1133- |
| | 1369, 1146-1386, 1146-1475, 1146-1587, 1191-1381, 1191-1549, 1203-1330, 1350-1563, 1352-1612, 1363-1638, |
| | 1395-1582, 1395-1594 |
| 46/2768535CB1/ | 1-520, 46-518, 46-520, 88-707, 129-472, 186-883, 187-765, 206-353, 421-1034, 505-1159, 623-908, 644-1251, 652- |
| 4790 | 1211, 654-1195, 678-1221, 690-1269, 703-1254, 704-1393, 706-989, 762-1001, 770-1352, 781-1389, 797-1335, 813- |
| | 1420, 870-1413, 945-1256, 1050-1703, 1108-1788, 1114-1738, 1132-1698, 1173-1815, 1189-1796, 1195-1801, 1204- |
| | 1798, 1220-1719, 1248-1823, 1262-1921, 1266-1924, 1270-1883, 1272-1807, 1283-1664, 1283-1870, 1336-2021, |
| | 1425-2056, 1438-2076, 1527-2070, 1621-2136, 1676-2224, 1696-2084, 1750-2313, 1863-2252, 1918-2152, 1923- |
| | 2186, 1926-2568, 1964-2230, 1964-2464, 2072-2606, 2275-2500, 2401-3007, 2464-2726, 2470-2676, 2494-2785, |
| | 2533-2819, 2540-2809, 2595-3128, 2621-2839, 2640-2882, 2649-2884, 2654-2925, 2656-2930, 2698-2958, 2727- |
| | 2928, 2727-3213, 2782-2953, 2782-3400, 2853-3148, 2858-3045, 2889-3189, 2891-3338, 2891-3379, 2894-3205, |
| | 2934-3187, 2948-3209, 2972-3515, 2986-3244, 3000-3209, 3003-3246, 3027-3291, 3028-3230, 3028-3300, 3035- |
| | 3298, 3074-3268, 3074-3298, 3074-3666, 3106-3350, 3192-3404, 3192-3456, 3221-3449, 3223-3640, |
| | 3266-3531, 3266-3756, 3289-3860, 3292-3804, 3305-3643, 3308-3608, 3309-3598, 3361-3649, 3364-3659, 3400- |
| | 3643, 3406-3654, 3407-3672, 3411-3811, 3420-3867, 3421-3695, 3424-3703, 3428-3705, 3450-3787, 3454-3696, |
| | 3460-3739, 3473-3868, 3473-3875, 3487-3648, 3498-3745, 3503-3767, 3516-3753, 3533-3728, 3539-3762, 3599- |
| | 3809, 3601-3733, 3620-4123, 3638-3878, 3647-3669, 3647-3884, 3647-3919, 3647-4186, 3716-3988, 3724-3996, |
| | 3737-4008, 3746-3998, 3752-3983, 3755-4415, 3777-4010, 3777-4404, 3809-4098, 3823-4098, 3836-4109, 3857- |
| | 4095, 3858-4065, 3863-4367, 3875-4093, 3882-4128, 3900-4146, 3909-4147, 3984-4413, 3995-4242, 4025-4670, |
| | 4043-4296, 4056-4349, 4086-4123, 4100-4398, 4100-4593, 4100-4688, 4115-4333, 4116-4594, 4143-4696, 4147- |
| | 4677, 4152-4410, 4191-4680, 4202-4429, 4215-4494, 4221-4479, 4222-4509, 4277-4532, 4318-4682, 4322-4659, |
| | 4328-4543, 4380-4630, 4394-4669, 4422-4661, 4425-4667, 4439-4696, 4444-4696, 4450-4665, 4459-4696, 4472- |
| | 4676, 4494-4696, 4494-4790 |
| 47/6848851CB1/ | 1-293, 136-477, 137-447, 170-482, 171-429, 171-477, 172-432, 172-446, 173-303, 173-433, 173-447, 174-416, 175- |
| 3916 | 443, 180-393, 193-365, 193-447, 197-447, 202-447, 204-476, 206-476, 257-599, 366-599, 366-749, 386-429, 502- |
| | 684, 509-743, 600-749, 600-1197, 604-1073, 604-1184, 627-1215, 627-1308, 750-1197, 750-1603, 775-951, 921- |
| | 1562, 1143-1704, 1198-1603, 1198-1737, 1244-1601, 1293-1712, 1365-1613, 1596-1748, 1596-2025, 1604-1737, |
| | 1604-1858, 1605-1997, 1680-2413, 1728-2255, 1732-2340, 1749-1997, 1782-2357, 2040-2689, 2040-2690, 2059- |
| | 2331, 2254-2690, 2421-2777, 2421-2794, 2424-2824, 2435-3135, 2465-2742, 2480-2802, 2551-3204, 2571-2852, |
| | 2609-3171, 2637-2912, 2732-3429, 2946-3149, 2946-3219, 2989-3258, 2994-3238, 3162-3406, 3186-3436, 3187- |
| | 3610, 3214-3590, 3214-3620, 3226-3767, 3232-3512, 3235-3493, 3235-3616, 3254-3916, 3280-3916, 3378-3607 |
| 48/7040722CB1/ | 1-1702, 5-561, 20-561, 107-167, 107-194, 107-240, 264-546, 420-852, 420-878, 420-1108, 420-1120, 424-1094, 947- |
| 1702 | 1702, 952-988, 953-1702, 956-1702, 965-1702 |
| 49/6430290CB1/ | 1-274, 27-274, 28-274, 29-274, 29-583, 40-682, 567-1426, 567-1429, 567-1448, 567-1458, 567-1462, 931-1211 |
| 1462 | |
| 50/2640251CB1/ | 1-282, 19-539, 73-658, 78-392, 83-598, 97-759, 106-624, 236-700, 335-611, 337-611, 375-634, 384-987, 404-759, |
| 3958 | 489-700, 508-825, 523-790, 528-947, 594-899, 600-899, 600-902, 605-899, 668-931, 678-925, 685-899, 703-947, |
| | 746-983, 780-1061, 843-1073, 881-1389, 977-1275, 1043-1287, 1046-1323, 1111-1692, 1231-1506, 1322-1572, |
| | 1379-1630, 1408-1814, 1408-1873, 1433-2210, 1440-1692, 1563-2005, 1614-2034, 1626-2024, 1732-2034, 1750- |
| | 1972, 1820-2378, 1853-2417, 1975-2249, 2009-2210, 2043-2287, 2084-2626, 2091-2619, 2454-3008, 2468-3078, |
| | 2480-3019, 2584-3143, 2678-3169, 2687-3270, 2754-3231, 2901-3169, 2902-3169, 2936-3597, 3261-3958, 3382- |
| | 3686, 3382-3779 |
| 51/3839350CB1/ | 1-296, 1-432, 1-450, 1-549, 1-563, 1-796, 3-360, 3-826, 102-826, 161-430, 190-798, 199-401, 221-736, 246-564, |
| 826 | 254-773, 272-489, 299-436 |
| 52/6393813CB1/ | 1-501, 18-719, 32-407, 32-493, 42-536, 51-302, 57-630, 58-305, 68-351, 68-608, 124-411, 173-710, 193-644, 208- |
| 729 | 232, 216-516, 230-463, 230-716, 243-706, 266-706, 278-536, 283-729, 321-729, 334-717, 538-729, 594-655 |
| 53/5685755CB1/ | 1-201, 76-697, 79-316, 197-478, 197-1448, 349-828, 351-803, 351-845, 353-846, 354-843, 358-845, 362-820, 363- |
| 1610 | 828, 365-846, 366-846, 434-846, 459-846, 476-787, 479-1448, 480-1553, 530-816, 546-846, 606-846, 609-828, 839- |
| | 1279, 839-1333, 840-1331, 843-1210, 865-1323, 874-1051, 882-1321, 1252-1610 |
| 54/71728459CB1/ | 1-337, 1-576, 1-617, 1-869, 120-487, 283-749, 283-750, 285-696, 286-739, 326-829, 327-828, 383-774, 420-819, |
| 869 | 456-869 |
| 55/1904303CB1/ | 1-515, 290-1414, 404-692, 416-828, 422-698, 429-676, 429-980, 530-747, 530-765, 530-1056, 586-858, 624-835, |
| 2209 | 744-1052, 761-1021, 780-1070, 793-1288, 877-1193, 976-1439, 978-1330, 1018-1382, 1057-1255, 1057-1397, 1096- |
| | 1378, 1097-1412, 1097-1414, 1173-1451, 1203-1325, 1247-1687, 1247-1690, 1248-2190, 1254-1539, 1258-1414, |
| | 1298-1773, 1339-1918, 1345-1614, 1345-1880, 1345-1918, 1399-1618, 1434-1836, 1459-2128, 1500-2115, 1517- |
| | 1747, 1523-1786, 1582-2168, 1587-2209, 1590-2179, 1693-2200, 1719-2165, 1722-2208, 1725-2190, 1761-2184, |
| | 1763-2188, 1772-2189, 1774-2182, 1779-2187, 1788-2156, 1796-2187, 1812-2208, 1818-2182, 1820-2208, 1822- |
| | 2182, 1823-2192, 1825-2187, 1827-2182, 1833-2182, 1834-2182, 1846-2190, 1849-2192, 1858-2190, 1861-2184, |
| | 1863-2192, 1870-2182, 1871-2182, 1878-2180, 1879-2160, 1879-2180, 1897-2182, 1907-2187, 1928-2208, 2004- |
| | 2167, 2095-2182 |
| 56/2911343CB1/ | 1-582, 1-1513, 43-138, 43-315, 43-573, 86-573, 101-347, 202-456, 465-569, 466-569, 523-996, 525-772, 528-1013, |
| 1520 | 571-831, 571-838, 573-1124, 578-1373, 620-849, 658-806, 666-931, 666-1181, 732-1273, 732-1368, 735-1345, 774- |
| | 1377, 800-944, 829-1410, 830-1400, 834-1104, 885-1146, 933-1210, 939-1520, 942-1460, 970-1191, 999-1274, |
| | 1000-1411, 1051-1506, 1053-1514, 1053-1520, 1066-1520, 1067-1520, 1068-1514, 1075-1470, 1083-1448, 1098- |
| | 1520, 1108-1423, 1111-1493, 1138-1514, 1152-1437, 1163-1520, 1180-1520, 1187-1514, 1260-1520, 1274-1514 |
| 57/7500308CB1/ | 1-1282, 4-295, 363-612, 363-888, 402-684, 402-707, 402-1050, 406-1001, 407-1018, 439-1006, 452-764, 454-859, |
| 1282 | 463-718, 474-1001, 495-1035, 516-976, 580-1045, 646-1278, 748-1046, 748-1050, 835-1116, 887-1282, 1011-1243, |
| | 1060-1282 |
| 58/7501098CB1/ | 1-252, 1-394, 1-397, 1-399, 1-1014, 87-193, 87-197, 87-198, 359-843, 363-843, 370-1010, 370-1016, 394-968, 410- |
| 1228 | 1004, 430-990, 460-713, 460-862, 460-1024, 463-996, 465-715, 465-1228, 469-916, 537-1016, 568-1019, 580-1018 |
| 59/7503839CB1/ | 1-128, 10-3582, 215-936, 270-739, 270-987, 293-883, 293-933, 293-1125, 441-617, 587-1256, 602-1405, 803-1370, |
| 3582 | 909-1423, 959-1382, 1014-1803, 1031-1279, 1081-1663, 1262-1414, 1262-1691, 1279-2186, 1346-1720, 1347- |
| | 1654, 1347-1700, 1347-1705, 1347-1720, 1347-1868, 1347-2098, 1394-1922, 1394-2009, 1415-1663, 1446-2023, |
| | 1706-2355, 1706-2356, 1710-2355, 1717-2355, 1725-1999, 1779-2355, 1888-2355, 1909-2355, 1920-2355, 1923- |
| | 2355, 1976-2284, 2002-2355, 2034-2947, 2087-2443, 2087-2460, 2090-2490, 2101-2801, 2137-2265, 2137-2408, |
| | 2147-2468, 2217-2870, 2237-2518, 2275-2837, 2303-2578, 2334-2745, 2340-3263, 2341-2615, 2341-2879, 2341- |
| | 2920, 2341-2927, 2341-3008, 2341-3037, 2341-3051, 2341-3082, 2341-3088, 2341-3134, 2341-3233, 2346-3199, |
| | 2352-3252, 2384-2822, 2398-3095, 2423-2696, 2488-2721, 2489-3363, 2562-3289, 2612-2815, 2612-2885, 2614- |
| | 3559, 2632-3296, 2660-2904, 2661-2924, 2674-3520, 2710-3484, 2828-3072, 2853-3102, 2853-3275, 2874-3555, |
| | 2880-3256, 2898-3178, 2901-3159, 2901-3282, 2920-3582, 2946-3582, 3049-3273, 3142-3546, 3258-3449, |
| | 3258-3561, 3373-3501 |
| 60/7503698CB1/ | 1-2043, 404-692, 416-828, 422-698, 429-676, 530-747, 530-765, 530-1056, 586-858, 624-835, 744-1052, 761-1021, |
| 2063 | 780-1070, 897-1056, 963-1267, 1100-1540, 1100-1543, 1107-1392, 1111-1267, 1147-1267, 1151-1626, 1192-1771, |
| | 1198-1352, 1198-1467, 1198-1733, 1198-1800, 1236-1984, 1252-1471, 1312-1606, 1312-1981, 1370-1600, 1384- |
| | 1609, 1435-2021, 1440-2043, 1443-2032, 1517-1809, 1518-1743, 1546-2043, 1572-2018, 1575-2043, 1578-2063, |
| | 1580-2037, 1584-2039, 1614-2037, 1616-2041, 1617-1993, 1625-2042, 1627-2035, 1632-2040, 1649-2040, 1665- |
| | 2043, 1671-2035, 1673-2043, 1675-2035, 1676-2035, 1678-2040, 1680-2035, 1681-2040, 1686-2035, 1687-2035, |
| | 1699-2046, 1711-2047, 1714-2037, 1716-2034, 1720-2031, 1720-2033, 1720-2039, 1720-2063, 1723-2035, 1724- |
| | 2035, 1724-2039, 1729-2039, 1731-2013, 1731-2033, 1731-2039, 1732-2013, 1732-2031, 1732-2033, 1732-2039, |
| | 1750-2035, 1760-2040, 1781-2043, 1857-2020, 1871-2063, 1948-2035, 1980-2043 |

TABLE 5

| Polynucleotide SEQ | | |
|---|---|---|
| ID NO: | Incyte Project ED: | Representative Library |
| 31 | 1895273CB1 | THP1NOT03 |
| 32 | 70072222CB1 | BRAIFEN05 |
| 33 | 3559223CB1 | BRAHTDK01 |
| 34 | 3441255CB1 | PLACNOB01 |
| 35 | 1958917CB1 | BRSTNOT23 |
| 36 | 6219465CB1 | BRAUNOR01 |
| 37 | 3576625CB1 | NERDTDN03 |
| 38 | 4765758CB1 | PLACNOT05 |
| 39 | 7236661CB1 | BRAUNOR01 |
| 40 | 7714187CB1 | BRAIHCT01 |
| 41 | 5136540CB1 | UTREDIT07 |
| 42 | 3277403CB1 | FIBRUNT02 |
| 43 | 1517569CB1 | LUNGNOT18 |
| 44 | 2415991CB1 | BRAINOT11 |
| 45 | 2735742CB1 | LUNGFET03 |
| 46 | 2768535CB1 | ADRENOT07 |
| 47 | 6848851CB1 | BRAIFER05 |
| 48 | 7040722CB1 | UTRSTMR02 |
| 49 | 6430290CB1 | LUNGNON07 |
| 50 | 2640251CB1 | BRAITUT03 |
| 51 | 3839350CB1 | DENDTNT01 |
| 52 | 6393813CB1 | KIDCTMT01 |
| 53 | 5685755CB1 | BRAIUNT01 |
| 54 | 71728459CB1 | BRAHNOT01 |
| 55 | 1904303CB1 | OVARTUT10 |
| 56 | 2911343CB1 | BSCNNOT03 |
| 57 | 7500308CB1 | BRAYDIN03 |
| 58 | 7501098CB1 | UTREDIT07 |
| 59 | 7503839CB1 | BRAIFER05 |
| 60 | 7503698CB1 | LUNGNOT02 |

TABLE 6

| Library | Vector | Library Description |
|---|---|---|
| ADRENOT07 | pINCY | Library was constructed using RNA isolated from adrenal |
| | | tissue removed from a 61-year-old female during a bilateral |
| | | adrenalectomy. Patient history included an unspecified |
| | | disorder of the adrenal glands. |
| BRAHNOT01 | pINCY | Library was constructed using RNA isolated from posterior |
| | | hippocampus tissue removed from a 35-year-old Caucasian |
| | | male who died from cardiac failure. Pathology indicated |
| | | moderate leptomeningeal fibrosis and multiple micro- |
| | | infarctions of the cerebral neocortex. Microscopically, |
| | | the cerebral hemisphere revealed moderate fibrosis of |
| | | the leptomeninges with focal calcifications. There was |
| | | evidence of shrunken and slightly eosinophilic pyramidal |
| | | neurons throughout the cerebral hemispheres. In addition, |
| | | scattered throughout the cerebral cortex, there were |
| | | multiple small microscopic areas of cavitation with |
| | | surrounding gliosis. Patient history included dilated |
| | | cardiomyopathy, congestive heart failure, cardiomegaly |
| | | and an enlarged spleen and liver. |
| BRAHTDK01 | PSPORT1 | This amplified and normalized library was constructed |
| | | using pooled RNA isolated from archaecortex, anterior and |
| | | posterior hippocampus tissue removed from a 55-year-old |
| | | Caucasian female who died from cholangiocarcinoma. Pathology |
| | | indicated mild meningeal fibrosis predominately over the |
| | | convexities, scattered axonal spheroids in the white |
| | | matter of the cingulate cortex and the thalamus, and a |
| | | few scattered neurofibrillary tangles in the entorhinal |
| | | cortex and the periaqueductal gray region. Pathology |
| | | for the associated tumor tissue indicated well- |
| | | differentiated cholangiocarcinoma of the liver with |
| | | residual or relapsed tumor. Patient history included |
| | | cholangiocarcinoma, post-operative Budd-Chiari |
| | | syndrome, biliary ascites, hydrothorax, dehydration, |
| | | malnutrition, oliguria and acute renal failure. Previous |
| | | surgeries included cholecystectomy and resection of 85% |
| | | of the liver. 7.6 × 10e5 independent clones from |
| | | this amplified library were normalized in 1 round |
| | | using conditions adapted Soares et al., PNAS (1994) |
| | | 91:9228-9232 and Bonaldo et al., Genome |
| | | Research (1996) 6:791, except that a significantly |
| | | longer (48 hours/round) reannealing hybridization |
| | | was used. |
| BRAIFEN05 | pINCY | This normalized fetal brain tissue library was |
| | | constructed from 3.26 million independent clones from |
| | | a fetal brain library. Starting RNA was made from brain |
| | | tissue removed from a Caucasian male fetus, who was |
| | | stillborn with a hypoplastic left heart at 23 weeks' |
| | | gestation. The library was normalized in 2 rounds |
| | | using conditions adapted from Soares et al., PNAS |
| | | (1994) 91:9228 and Bonaldo et al., Genome Research |
| | | (1996) 6:791, except that a significantly longer |
| | | (48 hours/round) reannealing hybridization was used. |
| BRAIFER05 | pINCY | Library was constructed using RNA isolated from |
| | | brain tissue removed from a Caucasian male fetus |
| | | who was stillborn with a hypoplastic left heart |
| | | at 23 weeks' gestation. |
| BRAIHCT01 | pINCY | Library was constructed using RNA isolated from |
| | | diseased occipital lobe tissue removed from the |
| | | brain of a 57-year-old Caucasian male, who died |
| | | from a cerebrovascular accident. Patient history |
| | | included Huntington's disease and emphysema. |
| BRAINOT11 | pINCY | Library was constructed using RNA isolated from |
| | | brain tissue removed from the right temporal lobe |
| | | of a 5-year-old Caucasian male during a |
| | | hemispherectomy. Pathology indicated extensive |
| | | polymicrogyria and mild to moderate gliosis |
| | | (predominantly subpial and subcortical), consistent |
| | | with chronic seizure disorder. Family history |
| | | included a cervical neoplasm. |
| BRAITUT03 | PSPORT1 | Library was constructed using RNA isolated from |
| | | brain tumor tissue removed from the left frontal |
| | | lobe of a 17-year-old Caucasian female during |
| | | excision of a cerebral meningeal lesion. Pathology |
| | | indicated a grade 4 fibrillary giant and small- |
| | | cell astrocytoma. Family history included benign |
| | | hypertension and cerebrovascular disease. |
| BRAIUNT01 | pINCY | Library was constructed using RNA isolated from |
| | | SK-N-MC, a neuroepithelioma cell line (ATCC HTB-10) |
| | | derived from a 14-year-old Caucasian female with |
| | | neuroepithelioma, with metastasis to the |
| | | supra-orbital area. |
| BRAUNOR01 | pINCY | This random primed library was constructed using RNA |
| | | isolated from striatum, globus pallidus and posterior |
| | | putamen tissue removed from an 81-year-old Caucasian |
| | | female who died from a hemorrhage and ruptured thoracic |
| | | aorta due to atherosclerosis. Pathology indicated |
| | | moderate atherosclerosis involving the internal |
| | | carotids, bilaterally; microscopic infarcts of the |
| | | frontal cortex and hippocampus; and scattered diffuse |
| | | amyloid plaques and neurofibrillary tangles, consistent |
| | | with age. Grossly, the leptomeninges showed only |
| | | mild thickening and hyalinization along the superior |
| | | sagittal sinus. The remainder of the leptomeninges |
| | | was thin and contained some congested blood vessels. |
| | | Mild atrophy was found mostly in the frontal poles |
| | | and lobes, and temporal lobes, bilaterally. |
| | | Microscopically, there were pairs of Alzheimer type II |
| | | astrocytes within the deep layers of the neocortex. |
| | | There was increased satellitosis around neurons in |
| | | the deep gray matter in the middle frontal cortex. |
| | | The amygdala contained rare diffuse plaques and |
| | | neurofibrillary tangles. The posterior hippocampus |
| | | contained a microscopic area of cystic cavitation |
| | | with hemosiderin-laden macrophages surrounded by |
| | | reactive gliosis. Patient history included sepsis, |
| | | cholangitis, post-operative atelectasis, pneumonia |
| | | CAD, cardiomegaly due to left ventricular hypertrophy, |
| | | splenomegaly, arteriolonephrosclerosis, nodular |
| | | colloidal goiter, emphysema, CHF, hypothyroidism, |
| | | and peripheral vascular disease. |
| BRAYDIN03 | pINCY | This normalized library was constructed from 6.7 |
| | | million independent clones from a brain tissue library. |
| | | Starting RNA was |
| | | made from RNA isolated from diseased hypothalamus |
| | | tissue removed from a 57-year-old Caucasian male who |
| | | died from a cerebrovascular accident. Patient history |
| | | included Huntington's disease and emphysema. The |
| | | library was normalized in 2 rounds using conditions |
| | | adapted from Soares et al., PNAS (1994) 91:9228 and |
| | | Bonaldo et al., Genome Research (1996) 6:791, except |
| | | that a significantly longer (48 -hours/round) |
| | | reannealing hybridization was used. The library |
| | | was linearized and recircularized to select for |
| | | insert containing clones. |
| BRSTNOT23 | pINCY | Library was constructed using RNA isolated from |
| | | diseased breast tissue removed from a 35-year-old |
| | | Caucasian female during a bilateral reduction |
| | | mammoplasty. Pathology indicated nonproliferative |
| | | fibrocystic disease. Family history included type |
| | | II diabetes, atherosclerotic coronary artery |
| | | disease, acute myocardial infarction, |
| | | hyperlipidemia, and coronary artery bypass. |
| BSCNNOT03 | pINCY | Library was constructed using RNA isolated from |
| | | caudate nucleus tissue removed from the brain of |
| | | a 92-year-old male. Pathology indicated several |
| | | small cerebral infarcts but no senile plaques or |
| | | neurofibrillary degeneration. Patient history |
| | | included throat cancer which was treated with |
| | | radiation. |
| DENDTNT01 | pINCY | Library was constructed using RNA isolated from |
| | | treated dendritic cells from peripheral blood. |
| FIBRUNT02 | pINCY | Library was constructed using RNA isolated from |
| | | an untreated MG-63 cell line derived from an |
| | | osteosarcoma removed from a 14-year-old |
| | | Caucasian male. |
| KIDCTMT01 | pINCY | Library was constructed using RNA isolated from |
| | | kidney cortex tissue removed from a 65-year-old |
| | | male during nephroureterectomy. Pathology for |
| | | the associated tumor tissue indicated grade 3 |
| | | renal cell carcinoma within the mid-portion of |
| | | the kidney and the renal capsule. |
| LUNGFET03 | pINCY | Library was constructed using RNA isolated from |
| | | lung tissue removed from a Caucasian female fetus, |
| | | who died at 20 weeks' gestation. |
| LUNGNON07 | pINCY | This normalized lung tissue library was constructed |
| | | from 5.1 million independent clones from a lung |
| | | tissue library. Starting RNA was made from RNA |
| | | isolated from lung tissue. The library was |
| | | normalized in two rounds using conditions |
| | | adapted from Soares et al., PNAS (1994) |
| | | 91:9228-9232 and Bonaldo et al., Genome |
| | | Research (1996) 6:791, except that a |
| | | significantly longer (48 hours/round) reannealing |
| | | hybridization was used. |
| LUNGNOT02 | PBLUESCRIPT | Library was constructed using RNA isolated from |
| | | the lung tissue of a 47-year-old Caucasian male, |
| | | who died of a subarachnoid hemorrhage. |
| LUNGNOT18 | pINCY | Library was constructed using RNA isolated from |
| | | left upper lobe lung tissue removed from a |
| | | 66-year-old Caucasian female. Pathology for the |
| | | associated tumor tissue indicated a grade 2 |
| | | adenocarcinoma. Patient history included |
| | | cerebrovascular disease, atherosclerotic coronary |
| | | artery disease, and pulmonary insufficiency. |
| | | Family history included a myocardial infarction |
| | | and atherosclerotic coronary artery disease. |
| NERDTDN03 | pINCY | This normalized dorsal root ganglion tissue |
| | | library was constructed from 1.05 million |
| | | independent clones from a dorsal root ganglion |
| | | tissue library. Starting RNA was made from |
| | | dorsal root ganglion tissue removed from the |
| | | cervical spine of a 32-year-old Caucasian male |
| | | who died from acute pulmonary edema, acute |
| | | bronchopneumonia, bilateral pleural effusions, |
| | | pericardial effusion, and malignant lymphoma |
| | | (natural killer cell type). The patient presented |
| | | with pyrexia of unknown origin, malaise, fatigue, |
| | | and gastrointestinal bleeding. Patient history |
| | | included probable cytomegalovirus infection, liver |
| | | congestion, and steatosis, splenomegaly, |
| | | hemorrhagic cystitis, thyroid hemorrhage, |
| | | respiratory failure, pneumonia of the |
| | | left lung, natural killer cell lymphoma of the |
| | | pharynx, Bell's palsy, and tobacco and alcohol |
| | | abuse. Previous surgeries included colonoscopy, |
| | | closed colon biopsy, adenotonsillectomy, and |
| | | nasopharyngeal endoscopy and biopsy. Patient |
| | | medications included Diflucan (fluconazole), |
| | | Deltasone (prednisone), hydrocodone, Lortab, |
| | | Alprazolam, Reazodone, ProMace-Cytabom, Etoposide, |
| | | Cisplatin, Cytarabine, and dexamethasone. The |
| | | patient received radiation therapy and multiple |
| | | blood transfusions. The library was normalized in |
| | | 2 rounds using conditions adapted from Soares et |
| | | al., PNAS (1994) 91:9228-9232 and Bonaldo |
| | | et al., Genome Research 6 (1996):791, except |
| | | that a significantly longer (48 hours/round) |
| | | reannealing hybridization was used. |
| OVARTUT10 | pINCY | Library was constructed using RNA isolated from |
| | | ovarian tumor tissue removed from the left ovary |
| | | of a 58-year-old Caucasian female during a total |
| | | abdominal hysterectomy, removal of a solitary ovary, |
| | | and repair of inguinal hernia. Pathology indicated |
| | | a metastatic grade 3 adenocarcinoma of colonic |
| | | origin, forming a partially cystic and necrotic |
| | | tumor mass in the left ovary, and an adenocarcinoma |
| | | of colonic origin, forming a nodule in the left |
| | | mesovarium. A single intramural leiomyoma was |
| | | identified in the myometrium. The cervix showed |
| | | mild chronic cystic cervicitis. Patient history |
| | | included benign hypertension, follicular cyst of |
| | | the ovary, colon cancer, benign colon neoplasm, |
| | | and osteoarthritis. Family history included |
| | | emphysema, myocardial infarction, atherosclerotic |
| | | coronary artery disease, benign hypertension, and |
| | | hyperlipidemia. |
| PLACNOB01 | PBLUESCRIPT | Library was constructed using RNA isolated from |
| | | placenta. |
| PLACNOT05 | pINCY | Library was constructed using RNA isolated from |
| | | placental tissue removed from a Caucasian male |
| | | fetus, who died after 18 weeks' gestation from |
| | | fetal demise. |
| THP1NOT03 | pINCY | Library was constructed using RNA isolated from |
| | | untreated THP-1 cells. THP-1 is a human promonocyte |
| | | line derived from the peripheral blood of a |
| | | 1-year-old Caucasian male with acute monocytic |
| | | leukemia (ref: Int. J. Cancer (1980) 26:171). |
| UTREDIT07 | pINCY | Library was constructed using RNA isolated from |
| | | diseased endometrial tissue removed from a |
| | | female during endometrial biopsy. Pathology |
| | | indicated in phase endometrium with missing |
| | | beta 3, Type II defects. |
| UTRSTMR02 | PCDNA2.1 | This random primed library was constructed |
| | | using pooled cDNA from two different donors. |
| | | cDNA was generated using mRNA isolated from |
| | | endometrial tissue removed from a 32-year-old |
| | | female (donor A) and using mRNA isolated from |
| | | myometrium removed from a 45-year-old female |
| | | (donor B) during vaginal hysterectomy and |
| | | bilateral salpingo- oophorectomy. In donor A, |
| | | pathology indicated the endometrium was secretory |
| | | phase. The cervix showed severe dysplasia |
| | | (CIN III) focally involving the squamocolumnar |
| | | junction at the 1, 6 and 7 o'clock positions. |
| | | Mild koilocytotic dysplasia was also identified |
| | | within the cervix. In donor B, pathology for the |
| | | matched tumor tissue indicated multiple (23) |
| | | subserosal, intramural, and submucosal leiomyomata. |
| | | Patient history included stress incontinence, |
| | | extrinsic asthma without status asthmaticus |
| | | and normal delivery in donor B. Family history |
| | | included cerebrovascular disease, depression, and |
| | | atherosclerotic coronary artery disease in donor B. |

TABLE 7

| Program | Description | Reference | Parameter Threshold |
|---|---|---|---|
| ABI | A program that removes | Applied Biosystems, | |
| FACTURA | vector sequences and | Foster City, CA. | |
| | masks ambiguous bases | | |
| | in nucleic acid sequences. | | |
| ABI/PARACEL FDF | A Fast Data Finder useful | Applied Biosystems, | Mismatch <50% |
| | in comparing and annotating | Foster City, CA; | |
| | amino acid or nucleic | Paracel Inc., Pasadena, CA. | |
| | acid sequences. | | |
| ABI | A program that assembles | Applied Biosystems, | |
| AutoAssembler | nucleic acid sequences. | Foster City, CA. | |
| BLAST | A Basic Local Alignment | Altschul, S. F. et al. | ESTs: Probability value = |
| | Search Tool useful in | (1990) J. Mol. Biol. | 1.0E−8 or less; Full |
| | sequence similarity search | 215:403-410; | Length sequences: |
| | for amino acid and nucleic | Altschul, S. F. et al. (1997) | Probability value = |
| | acid sequences. BLAST | Nucleic Acids Res. | 1.0E−10 or less |
| | includes five functions: | 25:3389-3402. | |
| | blastp, blastn, blastx, | | |
| | tblastn, and tblastx. | | |
| FASTA | A Pearson and Lipman | Pearson, W. R. and D. J. | ESTs: fasta E value = |
| | algorithm that searches for | Lipman (1988) Proc. | 1.06E−6; Assembled ESTs: |
| | similarity between a query | Natl. Acad Sci. USA | fasta Identity = |
| | sequence and a group of | 85:2444-2448; Pearson, W. R. | 95% or greater and Match |
| | sequences of the same type. | (1990) Methods Enzymol. | length = 200 bases or |
| | FASTA comprises as | 183:63-98; and Smith, | greater; fastx E value = |
| | least five functions: fasta, | T. F. and M. S. Waterman | 1.0E−8 or less; |
| | tfasta, fastx, tfastx, and | (1981) Adv. Appl. Math. | Full Length sequences: fastx |
| | ssearch. | 2:482-489. | score = 100 or greater |
| BLIMPS | A BLocks IMProved Searcher | Henikoff, S. and J. G. | Probability value = |
| | that matches a sequence | Henikoff (1991) Nucleic | 1.0E−3 or less |
| | against those in BLOCKS, | Acids Res. 19:6565-6572; | |
| | PRINTS, DOMO, PRODOM, | Henikoff, J. G. and S. | |
| | and PFAM databases to | Henikoff (1996) Methods | |
| | search for gene families, | Enzymol. 266:88-105; | |
| | sequence homology, and | and Attwood, T. K. et | |
| | structural fingerprint regions. | al. (1997) J. Chem. Inf. | |
| | | Comput. Sci. 37:417-424. | |
| HMMER | An algorithm for searching | Krogh, A. et al. (1994) J. | PFAM, SMART or TIGRFAM |
| | a query sequence against | Mol. Biol. 235:1501-1531; | hits: Probability |
| | hidden Markov model | Sonnhammer, E. L. L. et al. | value = 1.0E−3 |
| | (HMM)-based databases of | (1988) Nucleic Acids Res. | or less; Signal peptide hits: |
| | protein family consensus | 26:320-322; Durbin, R. et | Score = 0 or greater |
| | sequences, such as PFAM, | al. (1998) Our World View, | |
| | SMART and TIGRFAM. | in a Nutshell, Cambridge | |
| | | Univ. Press, pp. 1-350. | |
| ProfileScan | An algorithm that searches | Gribskov, M. et al. (1988) | Normalized quality |
| | for structural and | CABIOS 4:61-66; | score ≧ GCG specified |
| | sequence motifs in protein | Gribskov, M. et al. (1989) | “HIGH” |
| | sequences that match | Methods Enzymol. 183:146-159; | value for that particular |
| | sequence patterns | Bairoch, A. et al. (1997) | Prosite motif. Generally, |
| | defined in Prosite. | Nucleic Acids Res. 25:217-221. | score = 1.4-2.1. |
| Phred | A base-calling algorithm | Ewing, B. et al. (1998) | |
| | that examines automated | Genome Res. 8:175- | |
| | sequencer traces with high | 185; Ewing, B. and P. | |
| | sensitivity and probability. | Green (1998) Genome | |
| | | Res. 8:186-194. | |
| Phrap | A Phils Revised Assembly | Smith, T. F. and M. S. | Score = 120 |
| | Program including SWAT and | Waterman (1981) Adv. | or greater; Match |
| | CrossMatch, programs | Appl. Math. 2:482-489; | length = 56 |
| | based on efficient | Smith, T. F. and M. S. | or greater |
| | implementation of the | Waterman (1981) J. Mol. | |
| | Smith-Waterman algorithm, | Biol. 147:195- 197; and | |
| | useful in searching | Green, P., University of | |
| | sequence homology and | Washington, Seattle, WA. | |
| | assembling DNA sequences. | | |
| Consed | A graphical tool for | Gordon, D. et al. (1998) | |
| | viewing and editing Phrap | Genome Res. 8:195- | |
| | assemblies. | 202. | |
| SPScan | A weight matrix analysis | Nielson, H. et al. (1997) | Score = 3.5 |
| | program that scans protein | Protein Engineering | or greater |
| | sequences for the presence | 10:1-6; Claverie, | |
| | of secretory signal | J. M. and S. Audic (1997) | |
| | peptides. | CABIOS 12:431-439. | |
| TMAP | A program that uses weight | Persson, B. and P. Argos | |
| | matrices to delineate | (1994) J. Mol. Biol. | |
| | transmembrane segments | 237:182-192; Persson, | |
| | on protein sequences and | B. and P. Argos (1996) | |
| | determine orientation. | Protein Sci. 5:363-371. | |
| TMHMMER | A program that uses a | Sonnhammer, E. L. et al. | |
| | hidden Markov model (HMM) | (1998) Proc. Sixth Intl. | |
| | to delineate transmembrane | Conf. On Intelligent Systems | |
| | segments on protein | for Mol. Biol., Glasgow et | |
| | sequences and determine | al., eds., The Am. Assoc. | |
| | orientation. | for Artificial Intelligence | |
| | | (AAAI) Press, Menlo Park, CA, | |
| | | and MIT Press, Cambridge, | |
| | | MA, pp. 175-182. | |
| Motifs | A program that searches | Bairoch, A. et al. (1997) | |
| | amino acid sequences for | Nucleic Acids Res. 25:217-221; | |
| | patterns that matched | Wisconsin Package Program | |
| | those defined in Prosite. | Manual, version 9, page | |
| | | M51-59, Genetics | |
| | | Computer Group, Madison, WI. | |

TABLE 8

| | | | | | | | | | | Caucasian | African | | |
|---|---|---|---|---|---|---|---|---|---|---|---|---|---|
| SEQ | | | | | | | | | | Allele 1 | Allele 1 | Asian | Hispanic |
| ID | | | | EST | CB1 | EST | Allele | Allele | Amino | fre- | fre- | Allele 1 | Allele 1 |
| NO: | PID | EST ID | SNP ID | SNP | SNP | Allele | 1 | 2 | Acid | quency | quency | frequency | frequency |
| 57 | 7500308 | 6824818H1 | SNP00058395 | 72 | 952 | A | A | G | noncoding | n/d | n/a | n/a | n/a |
| 57 | 7500308 | 6824818H1 | SNP00142506 | 233 | 791 | A | A | G | noncoding | n/a | n/a | n/a | n/a |
| 57 | 7500308 | 6824818J1 | SNP00058395 | 580 | 952 | A | A | G | noncoding | n/d | n/a | n/a | n/a |
| 57 | 7500308 | 6824818J1 | SNP00142506 | 419 | 791 | A | A | G | noncoding | n/a | n/a | n/a | n/a |
| 59 | 7503839 | 1233581H1 | SNP00105660 | 163 | 3212 | A | A | G | noncoding | n/a | n/a | n/a | n/a |
| 59 | 7503839 | 1960883H1 | SNP00051538 | 159 | 3011 | C | C | T | noncoding | n/a | n/a | n/a | n/a |
| 59 | 7503839 | 2396122H2 | SNP00051538 | 111 | 3011 | C | C | T | noncoding | n/a | n/a | n/a | n/a |
| 59 | 7503839 | 2439231H1 | SNP00051538 | 184 | 3011 | C | C | T | noncoding | n/a | n/a | n/a | n/a |
| 59 | 7503839 | 3525859H1 | SNP00051538 | 114 | 3011 | C | C | T | noncoding | n/a | n/a | n/a | n/a |
| 59 | 7503839 | 5676172H1 | SNP00021725 | 41 | 1071 | C | C | A | noncoding | n/d | n/d | n/d | n/d |
| 59 | 7503839 | 6367830H1 | SNP00003533 | 249 | 3361 | C | C | T | noncoding | n/a | n/a | n/a | n/a |
| 59 | 7503839 | 7579662H1 | SNP00021725 | 113 | 1071 | C | C | A | noncoding | n/d | n/d | n/d | n/d |
| 59 | 7503839 | 7653343H1 | SNP00021725 | 485 | 1071 | C | C | A | noncoding | n/d | n/d | n/d | n/d |
| 59 | 7503839 | 7763335J1 | SNP00051538 | 85 | 3011 | C | C | T | noncoding | n/a | n/a | n/a | n/a |
| 59 | 7503839 | 8450847J1 | SNP00003533 | 257 | 3361 | C | C | T | noncoding | n/a | n/a | n/a | n/a |
| 59 | 7503839 | 8454173J1 | SNP00003533 | 257 | 3361 | C | C | T | noncoding | n/a | n/a | n/a | n/a |
| 59 | 7503839 | 8454612J1 | SNP00003533 | 255 | 3361 | C | C | T | noncoding | n/a | n/a | n/a | n/a |
| 60 | 7503698 | 1336111H1 | SNP00151238 | 61 | 1312 | A | A | G | E297 | n/a | n/a | n/a | n/a |
| 60 | 7503698 | 1904303H1 | SNP00151238 | 115 | 1312 | A | A | G | E297 | n/a | n/a | n/a | n/a |
| 60 | 7503698 | 3417040H1 | SNP00151239 | 194 | 1563 | C | C | T | P381 | n/a | n/a | n/a | n/a |
| 60 | 7503698 | 5943105H1 | SNP00151239 | 45 | 1561 | C | C | T | S380 | n/a | n/a | n/a | n/a |

1. An isolated polypeptide selected from the group consisting of:
a) a polypeptide comprising an amino acid sequence selected from the group consisting of SEQ ID NO:1-30,
b) a polypeptide comprising a naturally occurring amino acid sequence at least 90% identical to an amino acid sequence selected from the group consisting of SEQ ID NO:2, SEQ ID NO:4-5, SEQ ID NO:7-12, SEQ ID NO:14-22, and SEQ ID NO:24-30,
c) a polypeptide comprising a naturally occurring amino acid sequence at least 94% identical to an amino acid sequence selected from the group consisting of SEQ ID NO:1 and SEQ ID NO:3,
d) a polypeptide comprising a naturally occurring amino acid sequence at least 98% identical to an amino acid sequence of SEQ ID NO:6,
e) a polypeptide comprising a naturally occurring amino acid sequence at least 92% identical to an amino acid sequence of SEQ ID NO:13,
g) a polypeptide comprising a naturally occurring amino acid sequence at least 93% identical to an amino acid sequence of SEQ ID NO:23,
h) a biologically active fragment of a polypeptide having an amino acid sequence selected from the group consisting of SEQ ID NO:1-30, and
i) an immunogenic fragment of a polypeptide having an amino acid sequence selected from the group consisting of SEQ ID NO:1-30.

2. An isolated polypeptide of claim 1 comprising an amino acid sequence selected from the group consisting of SEQ ID NO:1-30.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:31-60.

6. A recombinant polynucleotide comprising a promoter sequence operably linked to a polynucleotide of claim 3.

7. A cell transformed with a recombinant polynucleotide of claim 6.

8. A transgenic organism comprising a recombinant polynucleotide of claim 6.

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. (canceled)

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:31-60,
b) a polynucleotide comprising a naturally occurring polynucleotide sequence at least 90% identical to a polynucleotide sequence selected from the group consisting of SEQ ID NO:32, SEQ ID NO:34-35, and SEQ ID NO:37-60,
c) a polynucleotide comprising naturally occurring a polynucleotide sequence at least 94% identical to the polynucleotide sequence selected from the group consisting of SEQ ID NO:31 and SEQ ID NO:33,
d) a polynucleotide comprising a naturally occurring polynucleotide sequence at least 98% identical to the polynucleotide sequence of SEQ ID NO:36,
e) a polynucleotide complementary to a polynucleotide of a),
f) a polynucleotide complementary to a polynucleotide of b),
g) a polynucleotide complementary to a polynucleotide of c),
h) a polynucleotide complementary to a polynucleotide of d), and
i) an RNA equivalent of a)-h).

13. (canceled)

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (canceled)

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO:1-30.

19. (canceled)

20. A method of screening a compound for effectiveness as an agonist of a polypeptide of claim 1, the method comprising:
a) exposing a sample comprising a polypeptide of claim 1 to a compound, and
b) detecting agonist activity in the sample.

21-22. (canceled)

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24-25. (canceled)

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. A method of screening for a compound that modulates the activity of the polypeptide of claim 1, the method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

28. (canceled)

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30-115. (canceled)


SEQUENCE LISTING (60 sequences)

SEQ ID NO: 1 - protein, 289 aa, Homo sapiens
  misc_feature: Incyte ID No 1895273CD1
MWFLTTLLLWVPVDGQVGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWNLEISLDSGHEKKVISSLQEDRHLEEELKCQEQKEEQLQEGVHRKEPQGAT

SEQ ID NO: 2 - protein, 159 aa, Homo sapiens
  misc_feature: Incyte ID No 70072222CD1
MSQAWVPGLAPTLLFSLLAGPQKIAAKCGLILACPKGFKCCGDSCCQENELFPGPVRIFVIIFLVILSVFCICGLAKCFCRNCREPEPDTPVDCRGPLELPSIIPPERVRVSLSAPPPPYSEVILKPSLGPTPTEPPPPYSFRPEEYTGDQRGIDNPAF

SEQ ID NO: 3 - protein, 559 aa, Homo sapiens
  misc_feature: Incyte ID No 3559223CD1
MAASEDGSGCLVSRGRSQSDPSVLTDSSATSSADAGENPDEMDQTPPARPEYLVSGIRTPPVRRNSKLATLGRIFKPWKWRKKKNEKLKQTTSALEKKMAGRQGREELIKKGLLEMMEQDAESKTCNPDGGPRSVQSEPPTPKSETLTSEDAQPGSPLATGTDQVSLDKPLSSAAHLDDAAKMPSASSGEEADAGSLLPTTNELSQALAGADSLDSPPRPLERSVGQLPSPPLLPTPPPKASSKTTKNVTGQATLFQASSMKSADPSLRGQLSTPTGSPHLTTVHRPLPPSRVIEELHRALATKHRQDSFQGRESKGSPKKRLDVRLSRTSSVERGKEREEAWSFDGALENKRTAAKESEENKENLIINSELKDDLLLYQDEEALNDSIISGTLPRKCKKELLAVKLRNRPSKQELEDRNIFPRRTDEERQEIRQQIEMKLSKRLSQRPAVEELERRNILKQRNDQTEQEERREIKQRLTRKLNQRPTVDELRDRKILIRFSDYVEVAKAQDYDRRADKPWTRLSAADKAAIRKELNEYKSNEMEVHASSKHLTRFHRP

SEQ ID NO: 4 - protein, 1222 aa, Homo sapiens
  misc_feature: Incyte ID No 3441255CD1
MPKGGAPPWIMALMFTGHLLFLALLMFAFSTFEESVSNYSEWAVFTDDIDQFKTQKVQDFRPNQKLKKSMLHPSLYFDAGEIQAMRQKSRASHLHLFRAIRSAVTVMLSNPTYYLPPPKHADFAAKWNEIYGNNLPPLALYCLLCPEDKVAFEFVLEYMDRMVGYKDWLVENAPGDEVPIVHSLTGFATAFDFLYNLLDNHRRQKYLEKIWVITEEMYEYSKVRSWGKQLLHNHQATNMIALLTGALVTGVDKGSKANIWKQAVVDVMEKTMFLLNHIVDGSLYEGVAYGSYTAKSVTQYVFLAQRHFNINNLDNNWLKMHFWFYYATLLPGFQRTVGIADSNYNWFYGPESQLVFLDKFILKNGAGNWLAQQIRKHRPKDGPMVPSTAQRWSTLHTEYIWYDPQLTPQPPADYGTAKIHTFPNWGVVTYGAGLPNTQTNTFVSFKSGKLGGRAVYDIVHFQPYSWIDGWRSFNPGHEHPDQNSFTFAPNGQVFVSEALYGPKLSHLNNVLVFAPSPSSQCNKPWEGQLGECAQWLKWTGEEVGDAAGEIITASQHGEMVFVSGEAVSAYSSAMRLKSVYRALLLLNSQTLLVVDHIERQEDSPINSVSAFFHNLDIDFKYIPYKFMNRYNGAMMDVWDAHYKMFWFDHHGNSPMASIQEAEQAAEFKKRWTQFVNVTFQMEPTITRIAYVFYGPYINVSSCRFIDSSNPGLQISLNVNNTEHVVSIVTDYHNLKTRFNYLGFGGFASVADQGQITRFGLGTQAIVKPVRHDRIIFPFGFKFNIAVGLILCISLVILTFQWRFYLSFRKLMRWILILVIALWFIELLDVWSTCSQPICAKWTRTEAEGSKKSLSSEGHHMDLPDVVITSLPGSGAEILKQLFFNSSDFLYIRVPTAYIDIPETELEIDSFVDACEWKVSDIRSGHFRLLRGWLQSLVQDTKLHLQNIHLHEPNRGKLAQYFAMNKDKKRKFKRRESLPEQRSQMKGAFDRDAEYIRALRRHLVYYPSARPVLSLSSGSWTLKLHFFQEVLGASMRALYIVRDPRAWIYSMLYNSKPSLYSLKNVPEHLAKLFKIEGGKGKCNLNSGYAFEYEPLRKELSKSKSNAVSLLSHLWLANTAAALRINTDLLPTSYQLVKFEDIVHFPQKTTERIFAFLGIPLSPASLNQILFATSTNLFYLPYEGEISPTNTNVWKQNLPRDEIKLIENICWTLMDRLGYPKFMD

SEQ ID NO: 5 - protein, 696 aa, Homo sapiens
  misc_feature: Incyte ID No 1958917CD1
MAMARLGSWLGEAQWLALVSLFVAALATVGLYLAQWALARARPQPQRRAVEPGEGPRPGSDALLSWILTLGSWRSQWQAAWVTALNEEAERKGGPPFLSFEEGPRQQALELVVQEVSSVLRSAEEKVVVCHVVGQAIQFLVSETPALGAGCRLYDMRLSPFHLQLEFHMKEKREDLQISWSFISVPEMAVNIQPKALGEDQVAETSAMSDVLKDILKHLAGSASPSVVLITKPTTVKEAQNLQCAASTAQESCPPKPPRAHELKLLVRNIHVLLLSEPGASGHINAVCVVQLNDPVQRFSSTLTKNTPDLMWEEEFTFELNAKSKELHLQISEAGRSSEGLLATATVPLDLFKKQPSGPQSFTLTSGSACGSSVLGSVTAEFSYMEPGELKSWPIPPPVPAAKIEKDRTVMPCGTVVTTVTAVKTKPRVDVGRASPLSSDSPVKTPIKVKVIEKDISVQAIACRSAPVSKTLSSSDTELLVLNGSDPVAEVAIRQLSESSKLKLKSPRKKSTIIISGISKTSLSQDHDAALMQGYTASVDSTHQEDAPSHPERAAASAPPEEAESAQASLAPKPQEDELDSWDLEKEPQAAAWSSQVLLDPDGDELSESSMSVLEPGTAKKHKGGILRKGAKLFFRRRHQQKDPGMSQSHNDLVFLEQPEGSRRKGITLTRILNKKLLSRHRNKNTMNGAPVEPCT

SEQ ID NO: 6 - protein, 436 aa, Homo sapiens
  misc_feature: Incyte ID No 6219465CD1
MGRARPGQRGPPSPGPAAQPPAPPRRRARSLALLGALLAAAAAAAVRVCARHAEAQAAARQELALKTLGTDGLFLFSSLDTDGDMYISPEEFKPIAEKLTGSTPAASYEEEELPPDPSEETLTIEARFQPLLPETMTKSKDGFLGVSRLALSGLRNWTAAASPSAVFATRHFQPFLPPPGQELGEPWWIIPSELSMFTGYLSNNRFYPPPPKGKEVIIHRLLSMFHPRPFVKTRFAPQGAVACLTAISDFYYTVMFRIHAEFQLSEPPDFPFWFSPAQFTGHIILSKDATHVRDFRLFVPNHRSLNVDMEWLYGASESSNMEVDIGYIPQVSAQEAPIQMELEATGPSVPSVILDEDGSMIDSHLPSGEPLQFVFEEIKWQQELSWEEAARRLEVAMYPFKKVSYLPFTEAFDRAKAENKLVHSILLWGALDDQSC

SEQ ID NO: 7 - protein, 652 aa, Homo sapiens [sequence omitted]

SEQ ID NO: 8 - protein, 91 aa, Homo sapiens [sequence omitted]

SEQ ID NO: 9 - protein, 155 aa, Homo sapiens [sequence omitted]

SEQ ID NO: 10 - protein, 765 aa, Homo sapiens
  misc_feature: Incyte ID No 7714187CD1
MIWRSRAGAELFSLMALWEWIALSLHCWVLAVAAVSDQHATSPFDWLLSDKGPFHRSQEYTDFVDRSRQGFSTRYKIYREFGRWKVNNLAVERRNFLGSPLPLAPEFFRNIRLLGRRPTLQQITENLIKKYGTHFLLSATLGGEESLTIFVDKRKLSKRAEGSDSTTNSSSVTLETLHQLAASYFIDRDSTLRRLHHIQIASTAIKVTETRTGPLGCSNYDNLDSVSSVLVQSPENKIQLQGLQVLLPDYLQERFVQAALSYIACNSEGEFICKENDCWCHCGPKFPECNCPSMDIQAMEENLLRITETWKAYNSDFEESDEFKLFMKRLPMNYFLNTSTIMHLWTMDSNFQRRYEQLENSMKQLFLKAQKIVHKLFSLSKRCHKQPLISLPRQRTSTYWLTRIQSFLYCNENGLLGSFSEETHSCTCPNDQVVCTAFLPCTVGDASACLTCAPDNRTRCGTCNTGYMLSQGLCKPEVAESTDHYIGFETDLQDLEMKYLLQKTDRRIEVHAIFISNDMRLNSWFDPSWRKRMLLTLKSNKYKSSLVHMILGLSLQICLTKNSTLEPVLAVYVNPFGGSHSESWFMPVNENSFPDWERTKLDLPLQCYNWTLTLGNKWKTFFETVHIYLRSRIKSNGPNGNESIYYEPLEFIDPSRNLGYMKINNIQVFGYSMHFDPEAIRDLILQLDYPYTQGSQDSALLQLLEIRDRVNKLSPPGQRRLDLFSCLLRHRLKLSTSEVVRIQSALQAFNAKLPNTMDYDTTKLC

SEQ ID NO: 11 - protein, 150 aa, Homo sapiens
  misc_feature: Incyte ID No 5136540CD1
MFTLLVLLSQLPTVTLGFPHCARGPKASKHAGEEVFTSKEEANFFIHRRLLYNRFDLELFTPGNLERECNEELCNYEEAREIFVDEDKTIAFWQEYSAKGPTTKSALQPSMKGGGTLPPSFSEDLRRLPCLHCRLLWRMQDYLLMNRQWR

SEQ ID NO: 12 - protein, 685 aa, Homo sapiens
  misc_feature: Incyte ID No 3277403CD1
MLWFSGVGALAERYCRRSPGITCCVLLLLNCSGVPMSLASSFLTGSVAKCENEGEVLQIPFITDNPCIMCVCLNKEVTCKREKCPVLSRDCALAIKQRGACCEQCKGCTYEGNTYNSSFKWQSPAEPCVLRQCQEGVVTESGVRCVVHCKNPLEHLGMCCPTCPGCVFEGVQYQEGEEFQPEGSKCTKCSCTGGRTQCVREVCPILSCPQHLSHIPPGQCCPKCLGQRKVFDLPFGSCLFRSDVYDNGSSFLYDNCTACTCRDSTVVCKRKCSHPGGCDQGQEGCCEECLLRVPPEDIKVCKFGNKIFQDGEMWSSINCTICACVKGRTECRNKQCIPISSCPQGKILNRKGCCPICTEKPGVCTVFGDPHYNTFDGRTFNFQGTCQYVLTKDCSSPASPFQVLVKNDARRTRSFSWTKSVELVLGESRVSLQQHLTVRWNGSRIALPCRAPHFHIDLDGYLLKVTTKAGLEISWDGDSFVEVMAAPHLKGKLCGLCGNYNGHKRDDLIGGDGNFKFDVDDFAESWRVESNEFCNRPQRKPVPELCQGTVKVKLRAHRECQKLKSWEFQTCHSTVDYATFYRSCVTDMCECPVHKNCYCESFLAYTRACQREGIKVHWEPQQNCAATQCKHGAVYDTCGPGCIKTCDNWNEIGPCNKPCVAGCHCPANLVLHKGRCIKPVLCPQR

SEQ ID NO: 13 - protein, 126 aa, Homo sapiens
  misc_feature: Incyte ID No 1517569CD1
MLLSSCLPPANVTTKAATPPPLVLSLTTADPAGKPAPCRVTLTLLRASIPATKRASFLSSFIKMFFEELEYILGFLSLLKFHVHVSVYSAICHFQKEGTGNSRSFTCTPELFPRLQTHLRAEGGAQ

SEQ ID NO: 14 - protein, 149 aa, Homo sapiens
  misc_feature: Incyte ID No 2415991CD1
MNYLFFFLTTSGLYCLSGSHGSNVKYIVLTYFNCSWSLTSPGFRDVLKGSQLWQVTDSWEMERTKEYSSCLTFLPTADIVQARVMEELNLLASQAAPIPTSQCTAPPHLFSPLSLTSPFIMSHKSGTVGSHYNLLCHRDSIFLISNHVS

SEQ ID NO: 15 - protein, 114 aa, Homo sapiens
  misc_feature: Incyte ID No 2735742CD1
MLSGARCRLASALRGTRAPPSAVARRCLHASGSRPLADRGKKTEEPPRDFDPALLEFLVCPLSKKPLRYEASTNELINEELGIAYPIIDGIPNMIPQAARMTRQSKKQEEVEQR

SEQ ID NO: 16 - protein, 519 aa, Homo sapiens
  misc_feature: Incyte ID No 2768535CD1
MTCPDKPGQLINWFICSLCVPRVRKLWSSRRPRTRRNLLLGTACAIYLGFLVSQVGRASLQHGQAAEKGPHRSRDTAEPSFPEIPLDGTLAPPESQGNGSTLQPNVVYITLRSKRSKPANIRGTVKPKRRKKHAVASAAPGQEALVGPSLQPQEAAREADAVAPGYAQGANLVKIGERPWRLVRGPGVRAGGPDFLQPSSRESNIRIYSESAPSWLSKDDIRRMRLLADSAVAGLRPVSSRSGARLLVLEGGAPGAVLRCGPSPCGLLKQPLDMSEVFAFHLDRILGLNRTLPSVSRKAEFIQDGRPCPIILWDASLSSASNDTHSSVKLTWGTYQQLLKQKCWQNGRVPKPESGCTEIHHHEWSKMALFDFLLQIYNRLDTNCCGFRPRKEDACVQNGLRPKCDDQGSAALAHIIQRKHDPRHLVFIDNKGFFDRSEDNLNFKLLEGIKEFPASAVSVLKSQHLRQKLLQSLFLDKVYWESQGGRQGIEKLIDVIEHRAKILITYINAHGVKVLPMNE

SEQ ID NO: 17 - protein, 1164 aa, Homo sapiens [sequence omitted]

SEQ ID NO: 18 - protein, 112 aa, Homo sapiens [sequence omitted]

SEQ ID NO: 19 - protein, 170 aa, Homo sapiens [sequence omitted]

SEQ ID NO: 20 - protein, 80 aa, Homo sapiens
  misc_feature: Incyte ID No 2640251CD1
MWCTCRCRTQQPGMPALMPSPRNAFSAEWLLALSSTCQHSSAASAPCQGVTRTRRIRKHGGVRKHWQSQHICEHVTHGLA

SEQ ID NO: 21 - protein, 118 aa, Homo sapiens [sequence omitted]

SEQ ID NO: 22 - protein, 140 aa, Homo sapiens
  misc_feature: Incyte ID No 6393813CD1
MAVVLPAVVEELLSEMAAAVQESARIPDEYLLSLKFLFGSSATQALDLVDRQSITLISSPSGRRVYQVLGSSSKTYTCLASCHYCSCPAFAFSVLRKSDSILCKHLLAVYLSQVMRTCQQLSVSDKQLTDILLMEKKQEA

SEQ ID NO: 23 - protein, 478 aa, Homo sapiens
  misc_feature: Incyte ID No 5685755CD1
MPRRGYSKPGSWGSFWAMLTLVGLVTHAAQRADVGGEAAGTSINHSQAVLQRLQELLRQGNASDVVLRVQAAGTDEVRVFHAHRLLLGLHSELFLELLSNQSEAVLQEPQDCAAVFDKFIRYLYCGELTVLLTQAIPLHRLATKYGVSSLQRGVADYMRAHLAGGAGPAVGWYHYAVGTGDEALRESCLQFLAWNLSAVAASTEWGAVSPELLWQLLQRSDLVLQDELELFHALEAWLGRARPPPAVAERALRAIRYPMIPPAQLFQLQARSAALARHGPAVADLLLQAYQFHAASPLHYAKFFDVNGSAFLPRNYLAPAWGAPWVINNPARDDRSTSFQTQLGPSGHDAGRRVTWNVLFSPRWLPVSLRPVYADAAGTALPAARPEDGRPRLVVTPASSGGDAAGVSFQKTVLVGARQQGRLLVRHAYSFHQSSEEAGDFLAHADLQRRNSEYLVENALHLHLIVKPVYHTLIRTPK

SEQ ID NO: 24 - protein, 80 aa, Homo sapiens [sequence omitted]

SEQ ID NO: 25 - protein, 505 aa, Homo sapiens
  misc_feature: Incyte ID No 1904303CD1
MSPWSWFLLQTLCLLPTGAASRRGAPGTANCELKPQQSELNSFLWTIKRDPPSYFFGTIHVPYTRVWDFIPDNSKEAFLQSSIVYFELDLTDPYTISALTSCQMLPQGENLQDVLPRDIYCRLKRHLEYVKLMMPLWMTPDQRGKGLYADYLFNAIAGNWERKRPVWVMLMVNSLTEVDIKSRGVPVLDLFLAQEAERLRKQTGAVEKVEEQCHPLNGLNFSQVIFALNQTLLQQESLRAGSLQIPYTTEDLIKHYNCGDLSSVILSHDSSQVPNFINATLPPQERITAQEIDSYLRRELIYKRNERIGKRVKALLEEFPDKGFFFAFGAGHFMGNNTVLDVLRREGYEVEHAPAGRPIHKGKSKKTSTRPTLSTIFAPKVPTLEVPAPEAVSSGHSTLPPLVSRPGSADTPSEAEQRFRKKRRRSQRRPRLRQFSDLWVRLEESDIVPQLQVPVLDRHISTELRLPRRGHSHHSQMVASSACLSLWTPVFWVLVLAFQTETPLL

SEQ ID NO: 26 - protein, 321 aa, Homo sapiens
  misc_feature: Incyte ID No 2911343CD1
MSGGKSAQGPEEGGVCITEALITKRNLTFPEDGELSEKMFHTLDELQTVRLDREGITTIRNLEGLQNLHSLYLQGNKIQQIENLACIPSLRFLSLAGNQIRQVENLLDLPCLQFLDLSENLIETLKLDEFPQSLLILNLSGNSCTNQDGYRELVTEALPLLLDLDGQPVVERWISDEEDEASSDEEFPELSGPFCSERGFLKELEQELSRHREHRQQTALTEHLLRMEMQPTLTDLPLLPGVPMAGDSSPSATPAQGEETVPEAVSSPQASSPTKKPCSLIPRGHQSSFWGRKGARAATAPKASVAEAPSTTKTTAKRSKK

SEQ ID NO: 27 - protein, 60 aa, Homo sapiens
  misc_feature: Incyte ID No 7500308CD1
MSQAWVPGLAPTLLFSLLAGPQKVILKPSLGPTPTEPPPPYSFRPEEYTGDQRGIDNPAF

SEQ ID NO: 28 - protein, 45 aa, Homo sapiens
  misc_feature: Incyte ID No 7501098CD1
MFTLLVLLSQLPTVTLGFPHCARGPKASKHAGEEDLIWSSSLPAT

SEQ ID NO: 29 - protein, 43 aa, Homo sapiens
  misc_feature: Incyte ID No 7503839CD1
MALFPAFAGLSEAPDGGSSRKVKEIMLQLILDIALFGLRTFRL

SEQ ID NO: 30 - protein, 456 aa, Homo sapiens
  misc_feature: Incyte ID No 7503698CD1
MSPWSWFLLQTLCLLPTGAASRRGAPGTANCELKPQQSELNSFLWTIKRDPPSYFFGTIHVPYTRVWDFIPDNSKEAFLQSSIVYFELDLTDPYTISALTSCQMLPQGENLQDVLPRDIYCRLKRHLEYVKLMMPLWMTPDQRGKGLYADYLFNAIAGNWERKRPVWVMLMVNSLTEVDIKSRGVPVLDLFLAQEAERLRKQTGAVEKVEEQCHPLNGLNFSQVPNFINATLPPQERITAQEIDSYLRRELIYKRNERIGKRVKALLEEFPDKGFFFAFGAGHFMGNNTVLDVLRREGYEVEHAPAGRPIHKGKSKKTSTRPTLSTIFAPKVPTLEVPAPEAVSSGHSTLPPLVSRPGSADTPSEAEQRFRKKRRRSQRRPRLRQFSDLWVRLEESDIVPQLQVPVLDRHISTELRLPRRGHSHHSQMVASSACLSLWTPVFWVLVLAFQTETPLL

SEQ ID NO: 31 - DNA, 1053 bp, Homo sapiens
  misc_feature: Incyte ID No 1895273CB1
ccagcttggagacaacatgtggttcttgacaactctgctcctttgggttccagttgatgggcaagtgggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgtaaagaactgaaaagaaagaaaaagtggaatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtagcagcggctcagtgggtggccatcgatctggaccgtcccctgcccacttgctccccgtgagcactgcgtacaaacatccaaaagttcaacaacaccagaactgtgtgtctcatggtatataactcttaaagcaaataaatgaactgacttcaaaaaaaaaaaaaaagg

SEQ ID NO: 32 - DNA, 1579 bp, Homo sapiens
  misc_feature: Incyte ID No 70072222CB1
agcccctgcagcagggtgggagcattaggacatgtccacacgtcttcaagggaaaatggctaagggctgtcagagaggcccagggaagaggcctgggtaggggcgagcagggagcagacaatcacttgttgaaggaagatccaagtccaggaaggaacgtagggcagtttggtgtcatggaaggaactgggggcccagccgaggggctcaagagcccttcagactctgcctgagcgagggtggtgctctgtcacccaggctggagtgtagtggtgtgatctcagctcactgcagccttgaactcctaggctcaagtgatcctcctacctcaacctccagagtaactgggactacaggaaagctcagtggcccccaagccaggatgtcccaagcttgggtccccggcctcgcgcccaccttgctgttcagcctgctggctggcccccaaaagattgcagccaaatgtggtctcatccttgcctgccccaaaggattcaaatgctgtggtgacagctgctgccaggagaacgagctcttccctggccccgtgaggatcttcgtcatcatcttcctggtcatcctgtccgtcttttgcatctgtggcctggctaagtgcttctgtcgcaactgcagagagccggagccagacaccccagtggattgccgggggcccctggaactgccctccatcatccccccagagagggtcagagtatccctttctgcgcccccacccccctacagtgaggtgattctgaagcccagcctgggcccaactcccacagagccaccccctccctacagcttcaggcctgaagaatataccggggatcagaggggcattgacaacccggccttctgagtcacctcctgcctggaatcttgccatcagcaacctcctccccagtgcctcctggatcaagctagagactgctggcaccccaggaatgtccctgcccatcctgccgtgtctctgttcattcttggatttaacttattactttttctgcttctgtttccaccccagctgcctctcttgtcctgagggttaggctggagtgacagtttccgcccaccccccagcccaagaaagaggctgccggaaagaaaatgctgaccattggaggtgcccaacagtagaatgggctactgtgaggggtagtaagagccccatttctggaggtatgcgaatcttgactggacagccagctctgagattttatcagggcacttctatacctgtgggacattggactggatgagccctgagccagcttccactcctacctgaatagagaactcactgcacccacccacaacacatgataaacacatgtcctcactgaatgttactgattgcggctgagggcctgcctctggctgtgtggggaggtgggtggagaggtgagcccaggcactgctgaggggtgcggtgatggggtcgctgcgccgcaatcccaccactgatgagccacctgggaggtctgggaggccagtccatccatgggccgccctcggagagaggcttgttctagatgtattggctgtctgttttttgatgtctctgtgtgccaaacag

SEQ ID NO: 33 - DNA, 2440 bp, Homo sapiens
  misc_feature: Incyte ID No 3559223CB1
agcagtccttgacgacgaggaatttttttttttttttttttttttttttttaattttcttttttaaaaagacgccccctccagccccctcgccggtgaccttggccgcctcggatgctctgattccacgcggctcgctctaacttgcccccgcgccggccgggcccatggccgcgtcggaggacgggagcggctgcctcgtgtcgcggggccgctcgcagagtgaccccagcgtcctcaccgactcctcggccacctcctccgcggacgccggggagaacccagatgagatggaccaaacgcccccggcgcgtcctgaatatctggtctcagggattcgaactccccctgtgaggaggaacagcaaactggccaccctgggcaggatcttcaaaccctggaaatggaggaaaaagaaaaacgaaaaactgaagcagacaacgtcagcgctggagaagaagatggccggcaggcaaggccgagaggagctcatcaagaaggggctgctggagatgatggagcaggatgctgaaagcaaaacttgcaaccccgatggaggaccccgatctgtacagagtgaaccacccactcccaagtcggagacgctgacttcagaagatgcccagcccggaagccccttggccactgggacggaccaggtctccctggacaagccactgtcctcagctgcccacttggacgatgcagccaagatgccttctgcatccagtggtgaagaagcagacgctggcagcctcctgcccaccaccaatgagctctcccaagccttagctggggctgactccctggacagtcctcccagacctctggagagatccgtgggccagctccccagccccccactgctgcccactccgccacccaaggcaagctccaaaaccacaaaaaatgtcacaggccaagccacactcttccaagcctccagcatgaagagtgccgacccttccctccggggccagctctccacacccacggggtctccgcatctcaccacggtccaccggcctcttcccccaagccgcgtcattgaggagctgcacagggcgctggccacgaagcaccgccaggacagttttcaaggaagagaaagtaaagggtctccaaagaagcggctggatgtccgtctgtcgagaacgtccagcgtggagcggggcaaggagagggaggaggcttggagctttgacggggcattggagaacaagcgaactgccgctaaggaatctgaggagaacaaggagaacctgatcataaattctgaactcaaagacgacttgcttttgtatcaggacgaggaggcgctgaacgactccattatttctggaacactgccacggaaatgcaagaaggagctcctggccgtgaagctaaggaaccggccaagcaaacaggaactagaagaccggaacattttccccagaaggactgatgaagaaagacaggagatccggcagcagatcgagatgaagctttccaaacggctgagccaaagacctgccgtggaagagctggagagaagaaatatcttgaaacaaaggaatgatcagacagagcaggaagaaagaagagaaatcaagcaaagattgacaagaaagcttaatcagagacccactgttgatgaattaagagacagaaaaattctgatacgattcagtgattacgtggaagtagcaaaagcgcaggactatgacaggagggcagacaaaccctggacgagactgtcagcagcagataaggcagcaattcgtaaagaattaaatgagtacaaaagtaatgaaatggaggtacatgcatcaagcaagcacttgacaagattccacaggccatagagattttcttctgagaagaatttgtgtttaattttttgataccaacactgaacattcatcagggaactttcctgaagttcagctcaagactaccctacctgctgtgtttgtgagaagagtaggatcacacacacaggtgcaatcttgaccacacttacctgcaagaggagtaaccagaggacacacttccttccttctttggtgtctgaggagtgtgaactgttggggtcagttaagacccaacataactctatcagaagaaaactgttgtttgcctttcaaccttgttttacagttctgcagtgtaatggaggacgggcaacgtgcatgtgcaggctcaccactcccaggcctctgacatgagggacatgtgacagtgtcattcagtattatgttcaaaagacatttttatcctgatcataattaatttgaaaactctttaagttcatgttatacaagatgatttactgtattatacttttccttttttatataatgtctaacaaaaaatacagctgcaacattttgattcctgttaattttgttctttaattaaatgactacttattgcaggaaaaaaaaaaaaaaaaaaaa

SEQ ID NO: 34 - DNA, 4133 bp, Homo sapiens
  misc_feature: Incyte ID No 3441255CB1
cttctttgggcaaacatacacaaatgcatcatacatgtgtggtgagcttatcaccagtgatggttttctgtgctagaaatgactcttaatttgaattttggagtgctttttctctttttttacaatgtgtgttccaactctttgtgttaaatagatttaagtaaaggaggtaaatgctaaattcatagtgttttttacctgtatcacttccctgtgtattatggaaaaattagagattttaacgttattcaaagttttactggaagcaaaactgtgccagggacagagatatacaatttaagttttctctttttggcaactgcacttgcttaaaatgtactgaatgtcagctggatttcacagcatatcagatttacagtctttgtcttatcaaggcctttactgtatgttttatactaaccagatgggaaacacattgagcatcatatctgacatgtatgcctaagggaggagctcccccatggatcatggcgttaatgtttacaggacatttactattcttagcattattgatgtttgctttctctacttttgaggaatctgtgagcaattattccgaatgggcagttttcacagatgatatagatcagtttaaaacacagaaagtgcaagatttcagacccaaccaaaagctgaagaaaagtatgcttcatccaagtttatattttgatgctggagaaatccaagcaatgagacaaaagtctcgtgcaagccatttgcatctttttagagctatcagaagtgcagtgacagttatgctgtccaacccaacatactacctacctccaccaaagcatgctgattttgctgccaagtggaatgaaatttatggtaacaatctgcctcctttagcattgtactgtttgttatgcccagaagacaaagttgcctttgaatttgtcttggaatatatggacaggatggttggctacaaagactggctagtagagaatgcaccaggagatgaggttccaattgtccattccttaacaggttttgccactgcctttgactttttatataacttattagataatcatcgaagacaaaaatacctggaaaaaatatgggttattactgaggaaatgtacgagtattccaaggtccgctcatggggcaaacagcttctccataaccaccaagccactaatatgatagcattactcacaggggccttggtgactggagtagataaaggatctaaagcaaatatatggaaacaggctgtagtggatgtcatggaaaagacaatgtttctattgaatcatattgttgatggttctttggatgaaggtgtggcctatggaagctacacagctaaatccgtcacacagtatgtttttctggcccagcgccattttaatatcaacaacttggataataactggttaaagatgcacttttggttctattatgccacccttttacctggcttccaaagaactgtgggtatagcagattccaattataattggttttatggtccagaaagccagctagttttcttggataagttcatcttaaagaatggagctggaaattggttagctcagcaaattagaaagcaccgacctaaagatggaccgatggttccttcaactgcccaaaggtggagtactcttcacactgaatacatctggtatgatccccagctcacaccacagccacctgctgattatggtactgcaaaaatacacacattccctaactggggtgtggttacttatggggctgggttgccaaacacacagaccaacacctttgtgtcttttaaatctgggaagctggggggacgagctgtgtatgacatagttcattttcagccatattcctggattgatgggtggagaagttttaacccaggacatgagcatccagatcagaactcatttacttttgcccccaatggacaagtatttgtttctgaagctctctatggacccaagttgagccaccttaacaatgtattggtgtttgctccatcaccctcaagccagtgtaataagccctgggaaggtcaactgggagaatgtgcgcagtggcttaagtggactggcgaggaggttggtgatgcagctggggaaataatcactgcctctcaacatggggaaatggtatttgtgagtggggaagccgtgtctgcttattcttcagcaatgagactgaaaagtgtatatcgtgctttgcttctcttaaattcccaaactctgctagttgttgatcatattgagaggcaagaagattccccaataaattctgtcagtgccttctttcataatttggatattgattttaaatatatcccatataagtttatgaataggtataatggtgccatgatggatgtgtgggatgcacattacaaaatgttttggtttgatcatcatggcaatagtcccatggccagtatacaggaagcagagcaagctgctgaatttaaaaaacgatggactcaatttgttaatgttacttttcagatggaacccacaatcacaagaattgcatatgtcttttatgggccatatatcaatgtctccagctgcagatttattgatagttccaatcctggacttcagatttctctcaatgtcaataatactgaacatgttgtttctattgtaactgattaccataacctgaagacaagattcaattatctgggattcggtggctttgccagtgtggctgatcaaggccaaataacccgatttggtttgggcactcaagcaatagtaaagcctgtaagacatgataggattattttcccctttggatttaaatttaatatagcagttggattaattttgtgcattagcttggtgattttaactttccaatggcgtttttacctttcttttagaaaactaatgcgatggatattaatacttgttattgccttgtggtttattgagcttttggatgtgtggagcacttgtagtcagcccatttgtgcaaaatggacaaggacagaggctgagggaagcaagaagtctttgtcttctgaagggcaccacatggatcttcctgatgttgtcattacctcacttcctggttcaggagctgaaattctcaaacaactttttttcaacagtagtgattttctctacatcagggttcctacagcctacattgatattcctgaaactgagttggaaatcgactcatttgtagatgcttgtgaatggaaggtgtcagatatccgcagtgggcattttcgtttactccgaggctggttgcagtctttagtccaggacacaaaattacatttgcaaaacatccatctgcatgaacccaataggggtaaactggcccaatattttgcaatgaataaggacaaaaaaagaaaatttaaaaggagagagtctttgccagaacaaagaagtcaaatgaaaggcgcctttgatagagatgctgaatatattagggctttgaggagacacctggtttactatccaagtgcacgtcctgtgctcagtttaagcagtggaagctggacgttaaagcttcatttttttcaggaagttttaggagcttcgatgagggcattgtacatagtaagagaccctcgggcatggatttattcaatgttgtacaatagtaaaccaagtctttattctttgaagaatgtaccagagcatttagcaaaattgtttaaaatagagggaggtaaaggcaaatgtaacttaaattcgggttatgctttcgagtatgaaccattgaggaaagaattatcaaaatccaaatcaaatgcagtgtccctcttgtctcacttgtggctagcaaatacagcagcagccttgagaataaatacagatttgctgcctactagctaccagctggtcaagtttgaagatattgtgcattttcctcagaaaactactgaaaggatttttgcctttcttggaattcctttgtctcctgctagtttaaaccaaatattgtttgccacctctacaaaccttttttaccttccctatgaaggggaaatatcaccaactaatactaatgtttggaaacagaacttgcctagagatgaaattaaactaattgaaaacatctgctggactctgatggatcgcctaggatatccaaagtttatggactaaaataaa

SEQ ID NO: 35 - DNA, 4689 bp, Homo sapiens
  misc_feature: Incyte ID No 1958917CB1
cgcgcgtttcctccccgcttgcccgcccagaggcccccggccacgctcctcgccgggctccgaccccgccgcgccccctccacgcccccagcgcgctgtggccgcagccagcgcgtgtcctgagtccgccgttccggcccggggctgccggggaagttggggcgaggcgggccggggatgcgccatggccatggcccggctgggctcgtggctcggggaggcgcagtggctcgcgctggtgtcgctcttcgtcgcggccctggccacggtaggcctgtacctggcgcagtgggcgctggccagggcgcgaccccagccccagcggcgggcggtggagcctggagaggggccgcgcccggggtccgacgcgctgctctcctggatcctgacgctgggcagctggaggagccagtggcaggcggcctgggtgaccgccctgaacgaggaggccgagaggaaagggggcccacctttcctgtcctttgaggagggcccgcggcagcaggcactggagctggtggtgcaggaggtctccagcgtgctcaggtcggcggaggagaaggtggtggtctgtcacgtggtgggccaggctattcagttcttggtcagcgagacgcctgccttgggtgctggatgccggctgtacgacatgcggctctcccctttccatttacagctggagttccacatgaaggagaagagagaggacctccagattagctggtctttcatcagtgtgccggaaatggccgttaatatccagcccaaagcactgggggaggaccaggtggctgagacaagtgcgatgtctgacgttctcaaggacatcttgaagcatttggctggttctgcctctccatcagtggttctgattaccaagcccacgactgtaaaggaagctcagaacttacagtgtgctgcatctactgctcaggaatcctgtcctcctaaacctccaagggctcacgagctgaagctactggtgaggaacatccacgtcttgctgctcagcgagcccggtgcctcaggccacattaatgcagtgtgcgtcgtgcagctgaacgatcctgttcagaggttctccagcaccctgacgaaaaacactccggacctcatgtgggaagaggaattcaccttcgagttgaatgccaagtcgaaggagttacacctgcagatttcagaggctgggcgatcctcagaaggtctgctggcgacggcgacagttcctctggacttatttaagaagcagccttctgggccacagagcttcacgctgaccagcgggtctgcctgcggcagctcggtgctgggctcggtcacggcagagttctcttacatggaacctggtgaattgaaatcctggcccatccctccccctgttcctgctgcaaaaatagaaaaggaccgcacggtgatgccctgtgggactgtggtcactactgtcactgctgtgaagaccaagcctcgcgtcgacgtggggagggcgtccccgctgagctctgattctccggtgaagactcccatcaaggtgaaggtgatcgagaaggacatctctgtccaggccatcgcctgccgcagcgcccccgtcagcaaaacactctcttcttcagacacagaattgttggtgttgaatggttcggatccagtggctgaagtggccattcgacagctcagtgaatcttcaaagctgaaactcaagtcgccacggaagaaaagcactattatcatatcagggatctccaagacctcactatctcaggaccacgacgctgccctgatgcagggctacacggcctctgtggacagcacccaccaggaggacgccccatcccatccggagagggcggcagcctctgccccgccagaggaagccgagtcagcccaggcatcccttgcccccaagccccaggaggacgagctagactcctgggacttggagaaggagccacaggccgcggcatggagcagccaggtcctgctggaccccgacggtgatgagctgtcagagagctccatgagtgtcttggagccgggcactgccaaaaagcataaaggaggaattctaaggaaaggtgcaaagctgttcttccgccggcggcatcaacagaaagacccaggcatgagtcagtcacacaatgaccttgtgttcctggagcagccagagggttcccggaggaaaggcatcaccctcaccaggatcctgaacaagaagctgctctccaggcacagaaacaagaacaccatgaacggtgcccccgtggagccctgcacgtagggcctgaggtcatcacctccaagccagaagacgtgcacccatgttaactaccctcaccaggacgcagccagtgtgtccgccggatgtccagatgccccgcttgtcttgctgggtttcttccaaccatctcgtcatttaaagggaaaacaaaatctgagtctccagccaggaggcttctcccagagaggacaaaaaagcccaacttgccaccagatgctaaatgagacttgacagctgcagagcttgggctgtgctcatagctaagggttagggttcaatattagaaggagattaacattataagtgaaataatatgctctaatagattgtggagggcaggtttgagggacttagtttacctattctacactaacaagtgttgttttgggtccatgcctggaccatgtcacaaaaaggaggtgcccccctgtgctgtcactgtgaatggaaaggatgggtcacctctcttcatctgctgcttggaataaaaaatgcagctggccctgagtacagggaagtggaacataggcaggattttggattaatagagaaattttgataagaatggagacgctacgacagatgtaggaagtcatctacctttgatattagccatagaacttgaacactaactatatcctatgcatagtatgcagaacacttttctaagtttactttgagcctacttgcaagtggaagatatatatattctcacatggtttttacatttttctctatcgtgttagaagctctaataatgctagtggagcagttgacatccagggttttttttcctgcctgtcatacttgctaaacaagagcacagcgggcctgtcagatgaagtcaggagccatacgtgaccgctcgtagaacacagtaaccaaacacatacatggattttgccaagtgctgccagtagccaaaacaaagtctttttagggcaatagaggaaattattttgtgtctcaggtgtcagtcttaggaatggaagtttaataacaaatgggccaaactcgcaggacattccttctatgagcgcttcagaattttgctgtgaacagtcctcttggacacaggttggggtgcccttgtttgggtttgttttggtggaaaacatcacaaacctggcacaccatttgaatatccctaatatcattccagtcgctttcctcatcagttgcctttctatttcagttcattcacagatctcacttctgaatgtgccacttccagtagacatgctggtcaaagagcagtcatcattggggtgaagtgttcttgacagtttaatatgattcacttttctccaaagacatgtaaaaggctgttacgaaagcttggcttctgtcatggagacggaaatgggcaagcttccttccgtagcctcttgttaatccttaaacattaaatatttcgggggtaatagagccactggtgagtaaaaacctatataaaaaccaaaattataggattttttcttttttagtaaaaacctgtatcaaaaccaaaattataggatttttttcttttttagtaaaaacctatataaaaaccaaaattataggattttttcttttttagtaaaaacctatatgaaaaccaaaattataggatttttttcttttttagtaaaaacctgtataaaaaccaaaattataggatttttttttcttcttttttagagagagagattagaaaacgacattaggaatttcactttaaaatgcgcattacaaacttcttaggtgttccaggaattatcaagtgactttaaaatgacttttccaacctgctttgtttttaaaaattatattccagttttaatcattgtaaaaaaagcacctggagtttcaaaacatgtgaatactaccaagtttctgtccccaaagtcaggcatcactgctagtcttttgggacagatgggacagatgttcactttaatgttttacttgaagttttactgctctttgccatgtggtaaaaagaggctgagacatatttaagaattccaagaggatattatgtgtcagaatttcagacactgatgagaagtttttaattgttcttttttatttgattttggaattcaggtgcactctattcaagtgcaaggatatcagaagtttttttttatttaaaaaatttttttttcgagatggagtttcactctgttgcccaggctggagtgcaatggcagcttactgcaacctccacctcctggttcaagcgattctcctgcctcagcctcccaagtagctgggattacaggcacgcgccaccacacctggctaattctatttagtagaaatggagtctcaccatgttggtcaggctggtctcgaactcctgacctcaggtgatccacccaccttggcacttacataaaccgcggtcgnccttgcggatttattat

SEQ ID NO: 36 - DNA, 3294 bp, Homo sapiens
  misc_feature: Incyte ID No 6219465CB1
atgggccgggcccggccgggccaacgcgggccgcccagccccggccccgccgcgcagcctcccgcgccaccgcgccgccgcgcccgttccctggcgctgctcggagccctgctggccgccgccgctgccgccgccgtccgggtctgcgcccgccacgccgaggcccaggcggccgcgcggcaggaactggcgctgaagaccctggggacagatggcctttttctcttttcctccttggacactgacggggatatgtacatcagccctgaggagttcaaacccattgctgagaagctaacagggtcaactcccgcggccagctacgaggaggaggagttgccccctgaccctagcgaggagacgctcaccatagaagcccgattccagcctctgctcccggagaccatgaccaagagcaaagatggcttcctaggggtctcccgcctcgccctgtccggcctccgaaactggacagccgccgcctcaccaagtgcagtgtttgccacccgccacttccagcccttccttcccccgccaggccaggagctgggtgagccctggtggatcatccccagtgagctgagcatgttcactggctacctgtccaacaaccgcttctatccaccgccgcccaagggcaaggaggtcatcatccaccggctcctgagcatgttccaccctcggccctttgtgaagacccgctttgcccctcagggagctgtggcctgcctgactgccatcagcgacttctactacactgtgatgttccggatccatgccgagttccagctcagtgagccgcccgacttccccttttggttctcccctgctcagttcaccggccacatcatcctctccaaagacgccacccacgtccgcgacttccggctcttcgtgcccaaccacaggtctctgaatgtggacatggagtggctttacggggccagtgaaagcagcaacatggaggtggacatcggctacataccccaggtgagcgcacaggaggctcccatccagatggagctggaggccacgggcccctctgtgccctccgtgatcctggatgaggatggcagcatgatcgacagccacctgccctcaggggagcccctgcagtttgtgtttgaggagatcaagtggcagcaggagctgagctgggaggaggctgcccggcgcctggaggtggccatgtaccccttcaagaaggtctcctacttgccgttcactgaggccttcgaccgagccaaggctgagaacaagctggtgcactcaatcctgctgtggggggccctggatgaccagtcctgctgaggttcagggcggactctccgggagactgtcctggaaagttcgcccatcctcaccctgctcaacgagagcttcatcagcacctggtccctggtgaaggagctggaggaactgcagaacaaacaggagaactcgtcccaccagaagctggctggcctgcacctggagaagtacagcttccccgtggagatgatgatctgcctgcccaatggcaccgtggtccatcacatcaatgccaactacttcttggacatcacctccgtgaagcccgaggaaatcgagagcaatctcttcagcttctcatccacctttgaagacccgtccacggccacctacatgcagttcctgaaggagggactccggcgtggcctgcccctcctccagccctagagtgcctggacgggatctgatgcacaggcccccacgcctcagagccagagtggtcctcagcccatttcagactgcagatgccgcccactcccaccccactcctaggctgccttggagggtacaagatccactgagggtggccaccacagccttggctccatggtggcgggtagacaagggatgcctgggctgactgggcagaggaacctctagctctgactgtcactcggctctccctacccatttggctctggaagctgcttggcccccccagatcagggcctgggtgaactccctggacctttcctagccagccgcacagtctaggcccttgtggggtgaagaatggagggaggagcaggctaggaagacggggccaccaccctctccttgctttcagcccttcccacaggaaacatcaagaagccccagccaggaggggccaggctgccaaggcggctcccctgtttatctagagccttcgttcctggccataccccggactgccctcctgtgcctgatgtccccagctggggtcagtctcaacaggagccagtcttctggagcctctgggcagaaccctccatcagagtggaaatcagacgggaccccctgcagcttccctgaccacgccactgaccagctatctggggaagtttactgtgaaggggtttctgcctttagcaatggggttcactaagggggttcccgaggcccagggccaaggcactcccaccgcctaccttagcacagggtctctgcaggactgcgggagccagcgctcctgccgcccctcttgcccctcagaccttgcatccacagaagcacaacccagccaaacaccacagccttctccagagccggcactgtcccggcaaccaggggtgccccaggctagctcttctacctctggggcaccacggactccccttggccactcttgggactttggtccacgtcctgagccactgaccacggccagtctctctttttatatgtgcagaaaagtgtttttacacaaactttctcatggtttgtaggtatttttttataaccccagtgctgaggagaaaggaggggcagtggcttccccggcagcagccccatgatggctgaatccgaaatcctcgatgggtccagcttgatgtctttgcagctgcacctatgggaagaagtagtcctctcttccttctcctcttcagctttttaaaaacagtcctcagaggatccatgatccccagcactgtcccatcctccacaaaggcccacaggcatgcctgtactctctttcattaaggtcttgaagtcaggctgccccctccccagcccccagttctctccccaccccctcaccccacccggggctcactcagcctggcagaggaagaaggaaggcagacatctccgcagccactcctgggccttttatgtgccgagttaccccacttgccttgggcgtgtccactgagccttccccagccagtcttgttctcaattttgttttgttttgttttgttttgagacggagtcttgctctgtcacccaggctggagtgctatggctcgatcttggctcactgcaacctccacctcccag

SEQ ID NO: 37 - DNA, 3635 bp, Homo sapiens
  misc_feature: Incyte ID No 3576625CB1
atggccgctgccgccctcccgccccggccgctgctccttctgccgctagtgctgctgctgagcggccgccccacgcgcgccgacagtaaggtgtttggggacctggaccaggtgaggatgacctcggagggctccgactgccgttgcaagtgcatcatgcggcccctgagcaaggacgcgtgtagccgagtgcgcagtgggcgggcacgcgtggaggacttctacacggtggagactgtgagctcgggcactgactgccgctgctcctgtaccgcacctccctcctctctcaacccctgtgagaacgagtggaagatggagaaactcaaaaagcaggcgcccgagctcctcaagctgcagtccatggtggatctcctggagggcaccctgtacagcatggacttgatgaaggtgcacgcctacgtccacaaggtggcctcccagatgaacacactggaagagagcatcaaggccaacctgagccgggagaatgaggtggtgaaggacagcgtgcgccacctcagtgagcagttgaggcactatgagaatcactctgccatcatgctgggcatcaagaaggagctgtcccgcctgggcctccagctgctgcagaaggatgccgccgccgcccctgccacccctgccacgggcactggtagcaaggcccaggacacagctagaggaaaaggcaaggacatcagcaagtatggcagtgtgcagaaaagctttgcagacagaggcctcccaaaacctcccaaggagaagctgcttcaggtggagaagctgagaaaggagagcggcaagggcagtttcctccagcccacagccaagccccgcgccctggcccagcagcaggctgtgatccggggcttcacctactacaaggcaggcaagcaggaggtgaccgaggcggtggcagacaacgccctccagggcacttcctggctggagcaactgccgcccaaggtggagggcaggtccaactccgcagagcccaactccgcagagcaggatgaggctgagcccaggtcctccgagcgagtggacctggcttctggcacccccacttcaatccctgccaccaccaccaccgccaccaccaccccaacccccaccaccagtctcctgcccaccgagccaccttcaggtccagaagtctccagccaaggcagagaggcgagctgtgagggcaccctccgggctgtggacccccctgtgaggcaccacagctatgggcgccacgagggagcctggatgaaggaccctgcagctcgagacgacaggatctatgtcaccaactactactatggaaacagcctggtggagttccgcaacctggaaaacttcaagcaaggccgctggagtaacatgtacaagctaccctacaactggatcggcacaggccacgtggtgtaccagggcgccttctactacaaccgcgccttcaccaagaacatcatcaagtacgacctacggcagcgcttcgtggcctcctgggcgctgctgcccgacgtggtatatgaggacaccacaccttggaagtggcgcggacactcggacattgactttgccgtggatgagagcggcctgtgggtcatctaccccgccgtggacgaccgcgatgaggcccagcccgaggtgatcgtcctgagtcgcttggaccccggcgatctctccgtgcaccgggagaccacgtggaagacacggctgcggcggaactcctacgggaactgcttcctggtgtgcggcatcctgtatgccgtggacacgtacaaccagcaggaaggccaggtcgcctacgctttcgacacgcacacgggcaccgacgcacgcccccagctgccgttcctcaacgagcacgcctacaccacccagatcgactacaaccccaaggagcgggtgctgtacgcctgggacaatggccaccagctcacctacaccctccacttcgtggtctgagtggagacctgtgctcccgggagaggggcagcagtgcgggaggggctttgcacagcagctcctgcaactgacccagtccgcaaatatttattgggggccagcccagggctgggactgggcatgaggtggtcaccaggattgagcttcctcagcacccagtgggtaatacttgcttccacttgcagagcaccgtgccaagcacttcccacacacttacccgtttgattctcctagcacctcccttggaggtagagatcatgaacccatttaacagacgaggagacaggctcagagaggcaccgtcccttgcctaacacctcagttgtgatcaggcaggctgtgctctcaggacagccccattttagggatgaggagacttcaccacgccctcccctccctgccctcccccatctcccctggtctcccttgttctctcaacccagtctcccttcccagggccactcagaaccagaggtctttagggccagtgtactggtgtggggtggaggccctggctctgcctgccatcctagggccctgttctggctgagctgttggtggccctgggctttgggccccttagccaatgtccttgtctcttgtctttggccagccccctcagcccagcccaccccacccgctgtccggccacattccaaacctctaccgtcacctagctgctgagcagaaaccgcaccccgagagaaaatcccatcctctgttccaaggcccctgtctgctctatgctcatttttattttctcttattcttcatcagtgccgtcatttgtttctgcagcagcagctgagaaggcagccggcagctctgccagggtggggagctgagctgaggctccctctccacccagaagcactggcgttgttcacatagtcaggccttgggtcccctccctggttcatcccagagcctttgggcctggagtccgccttgtcctttttctctgggctttcaaacccacaacctttacacactcagggatacctccgggtctgccatgaataagaccctaggcccaagtctggtgtggtgccaggatggcacagtttccctcttccttgccagccctgacctggtcactgggcaggctggcccagcagcctgggggctgcagaagacatggtgtgagtagttgggtccaggggaggcatcaggccttcttctggttgcaggagagaaccagagggtgggaacggggagggaagaactgagggtctgcagactggacttttcctggctcgacccaggacttgggttgaggatgcacgggggccaccttgcccggggccactggtggctccccagagcctcagacccacacagcccagaggacgaggccttcaagcctgcccctcttctgcttttttagacagtatttttagagctggaaagaaattttctagcccaactccctgtttcatgaaagagaaaagaggctcagaaaagtttagagagcagctcagtgtcacactgggagctgggcacagccgatctccttccagaagggttctgtctgtatcctttattctccgcaccattcccatcacccgtgaagagaagcctgctgtcaccgcagcccccaagaaatagatgccctttcttgaattgcattttttaaaacaagaaagtttccccaccagtgaatgaaagtcttgtga

SEQ ID NO: 38 - DNA, 635 bp, Homo sapiens
  misc_feature: Incyte ID No 4765758CB1
caacaatgtaggttttaattttcacattttatagatgtggaaattgagagagaggttagctgacttacccaaagtcacagtaatatatcaaatgcagacaggatttgaacccaggactctagttctgttgtccgtggtttatgctcatccttcctgggctagaggagaccacagggcctccgttcacaggcataagacaagagcccagtttccagatctggggtacaaggatgatgcatacactgggaaaacttttaagatttctttcaagaacaagagtcctagagattcggcctggctgtgtctgtaagtaagaatgtaatggctctagagtgggaaattaaagttgctaagcaggtccttatccacatttaatttcacccttcctcacaatgacagcttttttctcacttgctagtgattaagtgcatggacttgaagtcatatgaacttggttggaaatgctgtttcatacccttactagctgcgtggtcataggtaagttacttaacctctctgagccctagctttctcatctgcagggctggtatctcacaggattgtgaagagtcagtgaggactgtgttgacatgtttatcctaggcacaggttgtcaggtcaacttagttgttcat

SEQ ID NO: 39 - DNA, 793 bp, Homo sapiens
  misc_feature: Incyte ID No 7236661CB1
ggcgcccgtggcttgtgtgtgtggtttcccttctggtgccctactcgggaggttgttgctttgccccatgacaggaaggatcagcgtttctcctcttagcaaactgtgaggctggatgcagagattcagatactgcttttgtagatgtggtcggggcatatctggttgtgttttctctcccatgtttcatggggaagtgaagctcccgtggcctcctttgggggcctttgcctctgtccgctggtgatacaggggtcggtatcagagacctcttggcacgaaaccttgtgggacccacattggggctctggctctgggcacagaaggatacaacaaatcaaagttcggcagcctgtgcttctgccattaagtaaaccctttcagtggctgcatttttctgatctgggtttaacctcaaagaggaagagctttgacgccgggttccagcttcctcatcctggtgagtgtgccgaagagggagaagcaacggaggtggacaaacggcacctgtcagaggcgaaagctgctgtcagattggcgaggaagtccttaaaatggttgtgtgctttccgccgggtggctgtgccatggcctctctggttggcaccttgatggcagcccgcccccaggtaccatgttgccatggcgtctccatgcaggagcacacccttcatgaccacagctccgggaagcagggtctctgttccttgtcccgagagacccacggaaaaaattcctcttcctcatggcaagagggactcctctcccacgggggaggctcacagacgtctgt

SEQ ID NO: 40 - DNA, 2741 bp, Homo sapiens
  misc_feature: Incyte ID No 7714187CB1
ctctgatggctgttctcctagtccccaagagttgaaccggagaggcttttactttgcaccaggtcgagaacgtgatcagccctttagagaaggaacctccttgtaacaggaattctgctgggaaacgccgtgtaggcactacctccgaagataagatgcatgtttggagctgtgtaaaatgcccactggctgtttgaaagaaggaaaaggtgactagggttgcaaattaaccttagtatcacttaaaaatattctatctagtcaaatgtacgtaagcaaagaagagagcaccaggatataaaactgccacagcagcttggaagacagatgattcagattttggggactttcttttgcgtgttacatagatttgtttgtcatcatgcagttaagcaggtgttgagggaaagctgagagaatgaaggctctaaatccccagtggaagcatgatatggcgaagcagagctggtgctgaattgttctctctgatggctctatgggagtggatagcactgagtcttcattgctgggttttagcggttgctgctgtttcggatcagcatgccacaagccccttcgactggctcctctctgataagggacccttccatcgctcacaggaatacacagattttgtggacagaagccggcagggatttagcacaagatacaagatatacagggagtttggccgctggaaagtaaataaccttgcagttgagagaagaaatttccttggctctcctctgcctcttgcccctgaattcttccgcaacataagacttttgggacgtcgacctacccttcagcaaatcacagaaaaccttatcaagaaatatgggacacatttcttgctatctgctactctgggaggagaggagtcactcacaatttttgtggacaagcggaagttgagcaaacgagctgaaggaagtgattccaccaccaatagctcttcggtcactctggagacgctacatcagctagccgcttcttatttcattgacagggacagcacccttcggagacttcaccacattcaaattgcatccactgccataaaggtaacagaaacacggactggtcctcttggctgcagtaactatgacaacctagattctgtcagttctgttctggttcagagtcctgagaataagattcagttgcaagggcttcaagtacttctcccagactatcttcaggaacgttttgtacaagcagctttgagctacattgcttgcaattcagagggagagtttatctgcaaggaaaatgactgctggtgtcactgtggtcccaaatttccagaatgcaactgcccctccatggacattcaagccatggaagagaatcttcttcgaataactgaaacctggaaagcttacaacagtgactttgaggaatcagatgaattcaagttatttatgaaaaggctacctatgaattatttcctcaacacatctactataatgcatttgtggacaatggattctaattttcagcgccgttatgaacaactggagaacagcatgaaacaacttttcctaaaggcgcagaaaattgtacacaagctttttagccttagcaagaggtgtcataaacaacccctcatcagcctgccaagacaaagaacctcaacctactggcttactcgcatccagtcttttctctactgcaatgagaacggcctcctaggcagcttttcagaagagacgcactcgtgcacgtgtccgaatgaccaggtggtctgcaccgcgttcctgccctgcacagtgggagacgcctctgcctgcctgacatgcgcaccagacaaccgcacccgctgcggcacctgcaacaccggctacatgctcagccaggggctctgcaagcctgaagtcgccgagtccaccgatcactatattggctttgaaactgacctgcaagatctcgagatgaaatatctgctgcagaaaacggacagacgaatagaagtccatgccatttttatcagcaatgacatgcgcctcaatagctggtttgatccctcctggcgtaagcggatgctcctcaccttgaagagcaataagtacaagtcaagtctggtccatatgattttgggtctctctttacagatttgcttaactaaaaacagcaccttggagccagtgttggctgtttatgtcaatcccttcggaggcagccactctgagagctggtttatgcctgtgaatgaaaacagctttccagactgggagcggactaagttggacctacccctgcagtgttataactggacattaactctggggaacaaatggaagacattttttgagacagtacacatctacctgagaagtcgcatcaagtccaatggtcccaatggtaatgagagcatttactatgaacctctggagtttattgacccttcccggaacctgggctatatgaaaatcaataacattcaagtgtttggctacagcatgcactttgaccctgaagcaattcgggacctgattttgcagctggactacccctatactcagggatcccaggattcagcacttttgcaactactagagatcagagaccgtgtaaataaactctccccacctggtcagcgtcgtctagatcttttctcttgcttgcttcgtcatagactcaagctgtctactagtgaggtggtgaggatccaatctgctctgcaggcgtttaatgccaaattgccaaacacaatggattatgacacgaccaaattatgt

SEQ ID NO: 41 - DNA, 1074 bp, Homo sapiens
  misc_feature: Incyte ID No 5136540CB1
gcgaaccccaggcccttcccaggtttgcgcgcggtggccatccagaccctgcggagagcgaggcccggagcgtcgccgaggtttgagggcgccggagaccgagggcctggcggccgaaggaaccgccccaagaagagcctctggcccgggggctgctggaacatgtgcggggggacacagtttgtttgacagttgccagactatgtttacgcttctggttctactcagccaactgcccacagttaccctggggtttcctcattgcgcaagaggtccaaaggcttctaagcatgcgggagaagaagtgtttacatcaaaagaagaagcaaactttttcatacatagacgccttctgtataatagatttgatctggagctcttcactcccggcaacctagaaagagagtgcaatgaagaactttgcaattatgaggaagccagagagatttttgtggatgaagataaaacgattgcattttggcaggaatattcagctaaaggaccaaccacaaaatcagctcttcagccgtctatgaaagggggaggcacactccctccatcattttcagaagacctgaggaggctgccttgtctccattgccgccttctgtggaggatgcaggattaccttcttatgaacaggcagtggcgctgaccagaaaacacagtgtttcaccaccaccaccatatcctgggcacacaaaaggatttagggtatttaaaaaatctatgtctctcccatctcactgactaccttgtcattttggtataagaaatttgtgttatttgataggccgggcatggtggctcatgcctgtaatcccagcactttgggaggccaggagttcgagaccagcctggccaacatggtgaaacccggtctctactaaaaattcaaaaattacctaggcgtcatggggcatgcctgtagtcccacctacttgggaggctgaagcaggagaattgctcgaacctgggaggcagaggttgcagtaagctgagatcacgccactgcattccagcctgggcgacagagcaagactccatctcaaaaataaaataaaaaaaaaaggg

SEQ ID NO: 42 - DNA, 2560 bp, Homo sapiens
  misc_feature: Incyte ID No 3277403CB1
gcggcagctgagcagaggcggcggcgcgggacctgcagtcgccaggattccctccaggtgacgatgctctggttctccggcgtcggggctctggctgagcgttactgccgccgctcgcctgggattacgtgctgcgtcttgctgctactcaattgctcgggggtccccatgtctctggcttcctccttcttgacaggttctgttgcaaaatgtgaaaatgaaggtgaagtcctccagattccatttatcacagacaacccttgcataatgtgtgtctgcttgaacaaggaagtgacatgtaagagagagaagtgccccgtgctgtcccgagactgtgccctggccatcaagcagaggggagcctgttgtgaacagtgcaaaggttgcacctatgaaggaaatacctataacagctccttcaaatggcagagcccggctgagccttgtgttctacgccagtgccaggagggcgttgtcacagagtctggggtgcgctgtgttgttcattgtaaaaaccctttggagcatctgggaatgtgctgccccacatgtccaggctgtgtgtttgagggtgtgcagtatcaagaaggggaggaatttcagccagaaggaagcaaatgtaccaagtgttcctgcactggaggcaggacacaatgtgtgagagaagtctgtcccattctctcctgtccccagcaccttagtcacatacccccaggacagtgctgccccaaatgtttgggtcagaggaaagtgtttgacctcccttttgggagctgcctctttcgaagtgatgtttatgacaatggatcctcatttctgtacgataactgcacagcttgtacctgcagggactctactgtggtttgcaagaggaagtgctcccaccctggtggctgtgaccaaggccaggagggctgttgtgaagagtgcctcctacgagtgcccccagaagacatcaaagtatgcaaatttggcaacaagattttccaggatggagagatgtggtcctctatcaattgtaccatctgtgcttgtgtgaaaggcaggacggagtgtcgcaataagcagtgcattcccatcagtagctgcccacagggcaaaattctcaacagaaaaggatgctgtcctatttgcactgaaaagcccggcgtttgcacggtgtttggagatccccactacaacacttttgacggtcggacatttaactttcaggggacgtgtcagtacgttttgacaaaagactgctcctcccctgcctcgcccttccaggtgctggtgaagaacgacgcccgccggacacgctccttctcgtggaccaagtcggtggagctggtgctgggcgagagcagggtcagcctgcagcagcacctcaccgtgcgctggaacggctcgcgcatcgcgctcccctgccgcgcgccacacttccacatcgacctggatggctacctcttgaaagtgaccaccaaagcaggtttggaaatatcttgggatggagacagttttgtagaagtcatggctgcgccgcatctcaagggcaagctctgtggtctttgtggcaactacaatggacataaacgtgatgacttaattggtggagatggaaacttcaagtttgatgtggatgactttgctgaatcttggagggtggagtccaatgagttctgcaacagacctcagagaaagccagtgcctgaactgtgtcaagggacagtcaaggtaaagctccgggcccatcgagaatgccaaaagctcaaatcctgggagtttcagacctgccactcgactgtggactacgccactttctaccggtcctgtgtgacagacatgtgtgaatgtccagtccataaaaactgttattgcgagtcatttttggcatatacccgggcctgccagagagagggcatcaaagtccactgggagcctcagcagaattgtgcagccacccagtgtaagcatggtgctgtgtacgatacctgtggtccgggatgtatcaagacctgtgacaactggaatgaaattggtccatgcaacaagccgtgcgttgctgggtgccactgtccagcaaacttggtccttcacaagggaaggtgcatcaagccagtcctttgtccccagcggtgacctttgtttcgatccttaagactctgaaatctggtgactttgacactgaagcggaagagccaatgaaggactgcagtatttgtgtgcccgattctgtaaacacacacacacagagtatatatgtgtatatatatatagatatattcaaaaacattgcatcatttatatgaactatagggggattattatatgtatattttttgctataagacatgtattgtttctaggatcctaacctgtaagccattgaacatgttgtataaatacaccaggtgtttttaatttaataaggtggcatgcagatacattggatagtgttaacatcacatacatttgtcatttttaaggaagttttctaagagccctcaattgcctgcctgtattaattttagttttgatcaggagttgggggatccactatttaacgccgcacccgtgt

SEQ ID NO: 43 - DNA, 5833 bp, Homo sapiens
  misc_feature: Incyte ID No 1517569CB1
ctcctctcagaaatcgatgagtgccggtctcagccgtgcctgcatgggggctcttgtcaggaccgcgttgctgggtacctgtgcctctgcagcacaggctatgagggcgcccactgtgagctggagagggatgagtgccgagctcacccgtgcagaaatggagggtcctgcaggaacctcccaggggcctatgtctgccggtgccctgcaggcttcgttggagtccactgtgagacaggtaggggctccctccagtgggccccacatgcagagcctgggcctctggaagatcagagaggaggtggggcatccccacattccctgctgggcaggccacctggggagagggacccccagggctgcggccaccttgggagggaggggtggaggtgagggtgctggggagggctgaggggcgggacctgcccactgcccctctctcctggcctccgcccctagactcctcctttcccttcttcccgctgtcctctcctccctcccccagactccccccttgcagcttgggcccactctctgggtgttctccagaggtggacgcctgcgactccagcccctgccagcatggaggccggtgtgagagcggcggcggggcctacctgtgcgtctgcccagagagcttcttcggctaccactgcgagacagtgagtgacccctgcttctccagcccctgtgggggccgtggctattgcctggccagcaacggctcccacagctgcacctgcaaagtgggctacacgggcgaggactgcgccaaagagctcttcccaccgacggccctcaagatggagagagtggaggagagtggggtctctatctcctggaacccgcccaatggtccagccgccaggcagatgcttgatggctacgcggtcacctacgtctcctccgacggctcctaccgccgcacggactttgtggacaggacccgctcctcgcaccagctccaggccctggcggccggcagggcctacaacatctccgtcttctcagtgaagcgaaacagtaacaacaagaatgacatcagcaggcctgccgtgctgctggcccgcacgcgaccccgccctgtggaaggcttcgaggtcaccaatgtgacggctagcaccatctcagtgcagtgggccctgcacaggatccgccatgccaccgtcagtggggtccgtgtgtccatccgccaccctgaggccctcagggaccaggccaccgatgtggacaggagtgtggacaggttcacctttagggccctgctgcctgggaagaggtacaccatccagctgaccaccctcagtgggctcaggggagaggagcaccccacagagagcctggccaccgcgccgacgcacgtgtggacccggcccctgcctccagcaaacctgaccgccgcccgagtcactgccacctctgcccacgtggtctgggatgccccgactccaggcagcttgctggaggcttatgtcatcaatgtgaccaccagccagagcaccaagagccgctatgtccccaacgggaagctggcgtcctacacggtgcgcgacctgctgccgggacggcggtaccagctctctgtgatagcagtgcagagcacggagctcgggccgcagcacagcgagcccgcccacctctacatcatcacctcccccagggatggcgctgacagacgctggcaccagggaggacaccaccctcgggtgctcaagaacagaccgcccccggcgcgcctgccggagctgcgcctgctcaatgaccacagcgcccccgagacccccacccagccccccaggttctcggagcttgtggacggcagaggaagagtgagcgccaggttcggtggctcacccagcaaagcagccaccgtgagatcacaacccacagcctcggcgcagctcgagaacatggaggaagcccccaagcgggtcagcctggccctccagctccctgaacacggcagcaaggacatcggaaacgtccctggcaactgttcagaaaacccctgtcagaacggaggcacttgtgtgccgggcgcagacgcccacagctgtgactgcgggccagggttcaaaggcagacgctgcgagctcgcctgtataaaggtgtcccgcccctgcacaaggctgttctccgagacaaaggcctttccagtctgggagggaggcgtctgtcaccacgtgtataaaagagtctaccgagttcaccaagacatctgcttcaaagagagctgtgaaagcacaagcctcaagaagaccccaaacaggaaacaaagtaagagtcagacactggagaaatcttaaggatttaagacgttcttgttacactccaccaacctcacgagtttctaacacccaggaagatgaggtctaaaaactggatgaaaaaggacaccctgagaaaaggtcctagctggagtcagtcccctctgtgacctctctcctcaggcctctagaggacagatggccaggcctgtgcacacaccagcccaccctgagagacccctctgggaccaaccacctgtgagtcctgcgatgcgtttaagcagcctgtgccctcacccaagctgcagttcctgaaggtgtagtctgtgtctctgcggatgagatgacagctcgccattccccggaatcagtgaggctgtcagtcagccacgcttctgcagtatgcagaaacctgttcttagactccaaagccagagaaagaattctcccttcgaggcccaacaaattgagaaggaactgtgatggaccacttccaaaacagagacgggggcaggggctgaagggcagagaccaggtgatgtcagaaggaaagccgggttgcagacacagccgcccctgctctggtcctccagcgtgtttatgacgctcgtgcaggtcgacgagccatcctatggactagttaacactaaggtggagttcagacttttttagacaacggcgcgactggcagcctttctctatcaagggtcagacggtaaacgttttcagctttgcagaccagaggtccctgtggctacagtagcgcagacacagccacaggcatgtcattgaatggctgcggctatgttccaataaaaacttatttacaataacaggtggtggccaaattggcccatgggccttatttggtgaaccctgttctatgagatcacctaggcttcagccttaaacagtggaagccatcccctgaatgacaagtcacaagggtatcaaagaaagacccctgaattttcatggaaaaagctattcagacccctgcttggaaagctaaggcacactgccacgaagcagcaaggacgccttacaagtctcagtgcaacagagatggacacctgggctgggctggacaatgtttaaggttccttttagtccatgactcaagtgatactgttttaggctatcaggtagtaaacacgatcttagacatccccatctttgtaagcagaacagtacggcacttcaccacatctgcttcccaccatgcttctaagcagctgtcttccccctgctaatgttacaaccaaagcagccaccccacctcctctcgtgttgagcctcacgaccgctgacccagctggaaagccagcgccctgccgcgtcaccctgactctgctcagagccagcattccagccacaaagagggcctccttcctttcctctttcataaaaatgttttttgaagagttagagtatattttaggctttttatctttattaaaatttcatgtgcatgtgtctgtgtattctgcaatttgtcattttcagaaagaaggaacaggcaattcgcgaagtttcacctgtactcccgagctgttccccaggctccagacccacttgagagcagaaggtggagctcaatgaagggtctcgagctcagcgaagggtcactgggtgaactgagagaaaccacgttcacaaacgcgtactgcggacttcctgccgccctgggacctgtcactgtttgtccatgtaagctacagcattactagcagatgctaagatcgagtgatatcactggaaaagtaggtgaatcctactaggaaactttctactccctactaggacctcaagcccctcagccacacagcaaatgctaatatgctccagtgttagcttagaagccttgtgtcaacaagaactggctcctgagtcccaagcttggtgccacacagcaaatgctaatatgctccagtgttagcttagaagccttgtgtcaacaagaactggctcctgagtcccaagcttggtgccacacagcaaatgctaatatgctccagtgttagcttagaagccttgtgtcaacaagaactggctcctgagtcccaagtgctgtcacaggacttgcccattgggatgttttccacattaaatatcaagtaaaaagacttcctggtgctcaggaattacagttcgttcttgaaacattccaaagaggccaccacagcttttcccatgtggcttcttttaaaaactcaaatggcttccttgaaaatactcaaagtccacccaaggaaattagtaataatagaatcagaaaactgtcaggagcataaagatttctgtatcaaaatgaaagaagcaatcctgagttgctgaattacccatctgctaatgaaaccgggatggactgatcatctaaccaagtgcagactgaggattctacttagtcctccgactgggtacaacaacagcctaggttctagggagggtggcagtgaccgggatgccacaatggaagagaaaatgaaaacactggcacagtgaaatgtctcatttccaaacagttttgcctatggccaagcaaggcaataaagacaaactctcccttttccccattgcgtgtgggctgccaggtacaagtaagggaatctttgctgtgcccactgtcctccagtagagaacccaacaggcaagggccccactcaggtatcagctcacctcctgcacctccccttagcaggaactccttccactggcaaaggactgccactgccatctgactcaactgtggcgatgtggacggagtcaccgagctgcttttcttttgcaaaacaaaagtctttttctttgcagtcacgctgtaagacgaggctgctggagaaaacaaaagcacctagatttcagtgctgaatccccacaattgcatgcagctcacacctaccaggggtattccagtgcataggggaaaggaacccggctgaaaaaccagctccttatttttcttttaaataaaataatacgatcctaagtccatttaccatctgaagttgtcacgagtgaacagtcacattactgttgtggaccaggccttagatgagtttctcaggctcagcactgacattctgggccggatcatcctctcttgtgggaccatcctgtgcactgcaggatgtttcacagcacccctggcctctacccactagaatcctacaatctaccagatcctaggatctagttgatcctagaatgctaccaaggagacttgaattttggtcccatcatcaaaaatgccttctgcatcaatcctatgccagtttcccctaaaagagggctaactggaatgttctaggatgtaccaatcctccaggaccctcttagagctcatgccatcagagacaggcctctatctcagggatcaccccggctgacatcaaattcctcttctcttttcccaacatttcaaattgttcttcggactcattgagttccctaaggtgacaccccccccccccccccacacccaccttggggggtcacggattgctccctgtggccctggcttcagcccacctgctccatgaccccatgctcttctcctctggttc

SEQ ID NO: 44 - DNA, 2264 bp, Homo sapiens
  misc_feature: Incyte ID No 2415991CB1
gtaagccctaatatacactacataaaaacgttagggctgcctgtaatcccagcactttgggaagctgaggtgggtagatcacttgaggtcaggagttcgagaccagcctggccaacatggcgaaaccctgtctctactaaaaatacaaaattttagctgggtgttatggtgggcacctgtaatcccagctactttgaagatgaggtagcagactcacttgaaccagggaggcggaggttgcagtgagccaagattttgccactgcactccagcctgggtgacagagcaagactctgtctcacaaaaaaaaaaaaaaaagggggctgtacataggcagcaaactaagctgcagtgatgttgcctatatttaaattttctcaaatggccaagctctgatggtctactttatttgagcaatagttgagacttataattgcctataaataaacaaacaaatgaactatttgtttttttttctcacaacatctggcctatattgtctgtcaggaagccatggctccaatgtaaagtacatagttcttacatacttcaactgcagctggtccctgacctcaccaggtttcagagatgttcttaaaggaagccagctgtggcaggtcacagattcatgggaaatggaaagaaccaaggaatatagctcttgcctcacctttctacccactgcagatatagttcaagccagagtaatggaagaacttaacttactagcctctcaggctgctcctatccctacctcccagtgtacagcccctccccatctctttagtcccctttccctcacttccccttttataatgtcacacaaatcagggacagtaggatcacattataacctactttgtcatagggattcgatttttcttatatcaaatcatgtttcctgaaacccagctggggcatatgcactcaatgtctaatacatacttattaatgtaccggatattggccttgcccctggatatcagcaatatattataaaaggttccagtagatgagacgattgagtctgaatacaattgcagtaaattgtgccaataaagatattgtactgttacggtcttagagttaaagccgcttgaatgcagcatgcacattcatgtaaacagacaatcagggtaggcctagaataaccacaaaaattctattggccttactgcagccacctatatgtagaacaatggaggagatagtttgtggtccattattgtaccctgtttcatccattagcatcagaatctctctttcaggtcatttattaaatatgattgaaatgtttaaaagttcctgaacatgattcatgatgattaaaatatcatacaactgataaaagactttaagaactttatatatttcctgttgcctcaaaatgtaacagaaattattcttagagctttgattttagctatcctaattactgcaaataaatatttgttcttatagttttaaatcaaaaagaaaagtcttgttataaaaccttaagcttgaaatcatattaataaaatgtattgtacatagtggaaaattttcagtagctaatttaaaatttcagaaaatgctattaaagaattttgattcaagtatttaaactgtttagttatgcatgcttcttattaaccgaaaatgataataccatttagtttagtgatcagtatgagaagcaatacctaatcctatgttgctattgtattttttcctagttggtgtgcctgctcagaaaaacatatactgtatgtgtatacatacctgtgtatatataaaaggtcaatttatatatttttctataggaaaatggagtaacaagttccctatctcccatatttatttgtccatagtaaaatggccacattgatgataatttctagaactagtttctgagattgtcagccctttgtctaaaataatggcagtattaatgattgacttctgtcactgccatagttacctggattgtcagccttggtagcctttgtctaaagtcctaaagagttccaaaaaaaatgtgttgaaatttaattgctaaatagtggttggtgattctttacagtaggaattgtaataattttcttgcaaataagttatttactgctattgatattgaataatttgtcttttattcagatatatttcaaaaagcatgaatatatgattattcataaattgtatactttaccagtaagttttcagaggaaataaagacttttaaatccttaaaaaaaaaaaaaaaaaaaaaaaaaaaaaaaaaa

SEQ ID NO: 45 - DNA, 1638 bp, Homo sapiens
  misc_feature: Incyte ID No 2735742CB1
tcgagctggccactattacggcgcagtgtgctggaaagggtctgaatctaaagcagaggcagcataactatcttcacgactcctgtttgttgttgttgctttaaatctagaataccctgtcatgatggaaattgactataatacaactgtaattttaaatagcacattaaattattatgtacctatcacttttgtgcgtgctagtgtatcaaaaaggtattgttatacatatactaaatatatgacatttgagaaacactgttggtattttacttctgtagaataattcagatgagactaaaggccttataagtctacttattttgctgcggtgaggcctggtctccggctgccagaccatgctgagtggagcacgctgcaggctcgcctcagcgctgcggggaacgcgcgcgccgccgtccgcggtcgcccgtaggtgcctgcacgcgtcggggtcgcggcctttggccgaccggggcaagaagactgaggagccgccccgcgacttcgatccggcgctgctggagttcctggtgtgcccgctctccaagaagccgctcagatatgaagcatcaacaaatgaattgattaatgaagagttgggaatagcttatccaatcattgatgggatccctaatatgataccacaggcagctaggatgacacgtcaaagtaagaagcaagaagaagtggagcagcgctagttcataatttaaaaaaattaaaaaaacgcaacagccaacttttcttaataccatataccttttaaaacacagtggcaggtaataagtggaagagaagaatgtttctgtctcttcctacgttgactgttcttattccactggtttctttagcaggactgttctactcagcctctgtggaagaaaacttcccacagggctgcactagcacagccagcctttgcttttacagcctgctcttgcctattaccataccagtgtatgtattcttccacctttggacttggatgggtattaaactcttcaggcataattgatgcaactagagtcaatatgctgtatatattaatgatagctcttgggcatcgatctctgaaagctcaaatggatggaatttagtttgcgggaaagaggctttgctttgcgcatatcaggcttaggactgtgggaggcttaagttgcagatgcttcttttattgtactcttgttctgcccttgttttttgaaggctctgacttataactgctgtatcagaagaaacattttgacagtgtcttggttggagatgaacatccctaattgacatgtgatgactatttcttattccattcatctaagagtcattgaaattttgttttgcttgtttgtttagcttcaaggtctttggtaaagtcacatgttaaggatgactgaaataattccaaaggagtgatgttggaatagtccctctaagggagagaaatgcatttgaacgaatgtgatataaaaccacataatcaaatagaaacttcatgtacttacaaaaactgagtttgtaaaattaccttcatttctttgacattaaatgcttatattagcaataaacatgttgacactttcctataaaaaataaaccagtttgcagtaaaaaaaaaaaaaaagggcggccgccgactag

SEQ ID NO: 46 - DNA, 4790 bp, Homo sapiens
  misc_feature: Incyte ID No 2768535CB1
gacgtctttcttacctttttttctgaacttctaggccttctctttccagaactggtggaagacaaatgaaacggccaagatggtaagaaacaagccgcatttctccttggggagactgataatttaaaaggtttgttgtgtcagaaacattcccagcttcatcaccaaccctttccttccacctctgcccactggagaccacttacatcccgaagcggacgcggcagctgaagtcaggaaaccatgcatcacattagcaggagccaactgcagactttaaactccgttcaacatgtggatgcggcagagaaatgacctgtccagacaagccggggcagctcataaactggttcatctgctccctgtgcgtcccgcgggtgcgtaagctctggagcagccggcgtccaaggacccggagaaaccttctgctgggcactgcgtgtgccatctacttgggcttcctggtgagccaggtggggagggcctctctccagcatggacaggcggctgagaaggggccacatcgcagccgcgacaccgccgagccatccttccctgagatacccctggatggtaccctggcccctccagagtcccagggcaatgggtccactctgcagcccaatgtggtgtacattaccctacgctccaagcgcagcaagccggccaatatccgtggcaccgtgaagcccaagcgcaggaaaaagcatgcagtggcatcggctgccccagggcaggaggctttggtcggaccatcccttcagccgcaggaagcggcaagggaagctgatgctgtagcacctgggtacgctcagggagcaaacctggttaagattggagagcgaccctggaggttggtgcggggtccgggagtgcgagccgggggcccagacttcctgcagcccagctccagggagagcaacattaggatctacagcgagagcgccccctcctggctgagcaaagatgacatccgaagaatgcgactcttggcggacagcgcagtggcagggctccggcctgtgtcctctaggagcggagcccgtttgctggtgctggaggggggcgcacctggcgctgtgctccgctgtggccctagcccctgtgggcttctcaagcagcccttggacatgagtgaggtgtttgccttccacctagacaggatcctggggctcaacaggaccctgccgtctgtgagcaggaaagcagagttcatccaagatggccgcccatgccccatcattctttgggatgcatctttatcttcagcaagtaatgacacccattcttctgttaagctcacctggggaacttatcagcagttgctgaaacagaaatgctggcagaatggccgagtacccaagcctgaatcaggttgtactgaaatacatcatcatgagtggtccaagatggcactctttgattttttgttacagatttataatcgcttagatacaaattgctgtggattcagacctcgcaaggaagatgcctgtgtacagaatggattgaggccaaaatgtgatgaccaaggttctgcggctctagcacacattatccagcgaaagcatgacccaaggcatttggtttttatagacaacaagggtttctttgacaggagtgaagataacttaaacttcaaattgttagaaggcatcaaagagtttccagcttctgcagtttctgttttgaagagccagcacttacggcagaaacttcttcagtctctgtttcttgataaagtgtattgggaaagtcaaggaggtagacaaggaattgaaaagcttatcgatgtaatagaacacagagccaaaattcttatcacctatatcaatgcacacggggtcaaagtattacctatgaatgaatgacaaaagaatcttctggctagggtgttagatatatttatgcatttttggttttgtttttaaatcaagcacatcaacctcaagcccgtttagcaatgaggcagtgtagatgaatacgtaaaataaatgactttaaccaagtagctataatgggacttagcactgtatgcatacttaaaaaggttttgaaaaacaaactacttgagaaatatttgtttatatttttctctaacatcatgctatgtgtcagtctgaacatctgacaacagaaatttcagttattattctagctaagttttgaaaacatttgtcatgctgtttaatagaaaactgcaaaccagagatactgactccattaataaaccatattttgtgccgttttgactgttctgaccaaatactaatgggaacaattcttgacgtttttctgttgctgattgttaacatagagcagtctctacactaccctgaggcaactctacattggaacactgaggcttacagcctgcaagagcatcagagctgaccatacatttaaacagaaatgctggtttatttgcaaaatcaccagtatattttctattgtgtctataaaaaatcagtcatttaagtacaagaatcatattttccattcctttttagaaatttattttgttgtccctatggaaatcattcacatctgacaatttatatgttaaagagttttactctctctattttggtccaatttgtatctagtggctgagaaattaaataattctaaagtatgaagttacctatctgaaaatgtacttacagagtatcattttaaaatggatgtctctttaaaaattttgttacttttaccaacaatgtaatataatttatgtatattttattaataatagtgaattccttaaaatttgttctatgtacttatatttaatttgatttaatggttactgcccagatattgagaaatggttcaaatattgagtgtgtttcaatatattatctggcttatttcaacatgagtaatatgagcaaaataagttaaaacctgcgtctgatcaattttcctcatgactagaactaaaacagtaaatttggacaatattaagcctcaaataatcatctccaaactccttctaacactttttaaatcagattggaagacatggacaaatcaggttcatgtgttgcatctttatgtcctttgccaatatccaagatcatcacatatggtagatattcacatggagtttcaaattcagaatagattaccattaccttcctgcccttacacatcctactccttatttaaaagttctatttgtgacttttcatttcctgaaagtttaaaaatacaatttgagaatgtttataatacattctctcctgtcttttcacggttacgtctgttattgctgaaatacaccacattttctttgttctggtcaaggttaactcaatatctgtgtgaaagagaactactaacaacgttacaatagaggctagatttgaaaaaaaaaatctatagatctaattgatacaattgtagaacaaaatgtcaaaataatgttttaagtataagagaagatggaccaaggagagagagatcatttgaaaatctaattgtagcttttctaggctcacattcatgtactacttttagcacccttatgggctgtgctcgccccctggacagttgagctttggattatcttcctcttcaattttccctctattgacccgagtgtctccctctgcttctacagatttatagtactccttggctcttttgagtctccacttttactcactgtctctgggatttttaagatccttttcttctcttataaatcatcctcttaatgaaaattagcctaacaaaagtttggagactggaatcctactttgagccactgacttgaaataactcttttggcaagttgcctgacatcctgtcttaccaaggtggcatatttgcatttttactgcttaaaacatttttttttttttaccatctttatccaaatttatcatattgatggtaggactaacaggctttttagaagctggctttaactttgagtctcaagctacaatgctgttgggcagcctggtcttcccacgtgagggtttaactttgtttatttgcctccagttattccaaaatgcttattaaatgaaaggcccaggaacatgtttattttagtcacctttgctttttaacaattttgttttgtaatcaatgagtaattcatgatgaattatttttgactaatggatagccgaaggccaagcttttaattctaataggtaatgttcttcttttgtcttattgaaacaatgagaatactctgtgcatttcaaatgcactccgattatgctgtggttttattcacataagcacaatatgtgttttatttataacttcataacaaacttataatataataatttaccttagcagacatgcaaaagcttattcttgtgtgacttactttctttaagctaataatataaaaataaatatgtatcttaaaaatctataataaaacattagaaattaaagatatgtgctttttattttgcagatgagttcatttgcttctgtagatgtgttttcagagctaggtacagaggaatgtttgctacctttagcggtgaaaaaagaaagagagtcaagaattttgttggattgtgtttgtgtgtgcatatatttgatatcatcattatatttgtaatctttggacttgtaatcatagcctgtttattctactgtgccattaaatatactttaccttatacataacgaataaaatacctagatgtagatttatttacacactgtcttatgaaattcttatgtttatgtcagcacagaacaagcacacatttagagcattcatctgaanattatatggaaattgaccatgg

SEQ ID NO: 47 - DNA, 3916 bp, Homo sapiens
  misc_feature: Incyte ID No 6848851CB1
tgccccgccgcgccccagtcccttgacgaccctcctctctgggccccgcccctcccgcttcggggtcaagccccagagagcgccgcgaaaaccacatttcccagagtgcaccgcgacggcaggggtcctcagaccggcgctcgctcgccggcgccatccctatagagaagaacggaggtacggcctgtggtcatggcgctgttcccagcctttgcggggcttagtgaggctcccgatggcgggagctccaggaaagagttagactggctgagcaacccaagcttttgtgttggatccataacgtccctgagccaacaaactgaagcagctccagcccatgtttctgaagggttaccgctgacaaggagtcatctgaaatcagagtcttcagatgaaagtgacactaacaaaaagctcaaacaaacaagtagaaaaaagaagaaagagaaaaagaaaaaaaggaagcatcagcatcataagaaaacaaagaggaagcatgggccgtcgagtagcagcaggtctgagacagacaccgattctgaaaaggacaaaccttccagaggcgttggaggcagtaaaaaggaatctgaggaaccgaatcaaggaaataatgctgcagctgatactggacatcgctttgtttggcttgaggacattcaggctgtgacgggagaaaccttcagaacagataagaaaccagatcctgcgaactgggagtacaagtctctctaccgaggggatatagcaagatacaagaggaaaggagactcctgccttggcattaaccctaagaagcagtgcatatcttgggaagggacttccacagagaagaagcattcacgcaagcaggttgaacgctattttactaagaagagtgtgggattaatgaacatcgatggagttgccattagcagtaaaactgaacctccctcatctgagcccatctcctttataccagtgaaggacttggaagatgcggctcctgttacaacctggttgaatcctctggggatttatgatcagtcaaccacacattggctacaaggacagggtcctccagagcaggaatcaaagcagccagacgcacagccagacagcgagagtgcggctctcaaggccaaggtggaggagtttaacaggagggtgcgggagaatcctcgggatacgcagctgtggatggcatttgttgcttttcaggacgaggtcatgaaaagtcctggcctgtatgccatcgaggaaggagagcaggaaaagcgaaagaggtccctgaagctcattctggagaagaagctggccattctggagcgggccattgagagcaaccagagcagtgtggatctgaaactggccaagctgaagctctgcacagagttctgggagccctccactctggtcaaagagtggcagaaactgatatttttgcatcccaataatacagccctttggcagaaataccttttattttgccagagccagtttagtaccttttcgatatcaaaaattcacagtctttatggaaaatgcttgagcactttgtctgctgttaaggacggcagcatcttatctcaccctgcgttgcctggcacggaagaggccatgtttgcactctttcttcagcagtgccactttctgcggcaggctggccactctgagaaggccatctcattgttccaggccatggtggacttcaccttcttcaaacccgacagcgtgaaagatctgcctaccaaaggacaggtggaattctttgaacccttttgggacagtggagagccccgggctggggagaagggagcccgaggctggaaggcgtggatgcaccagcaggaacgaggtggctgggtggtcatcaacccagatgaggatgacgatgaaccagaagaggatgaccaggaaataaaagataagactctgcccaggtggcagatctggcttgctgctgagcgttcccgtgaccagaggcactggcggccctggcgccctgataagaccaagaagcaaaccgaggaagactgtgaggatcccgagagacaggtgttgtttgatgatattgggcaatctttgatcagactttccagccatgatcttcagttccagctggtggaggccttcctgcagttcttgggtgtgccttctggctttactcctccagcctcctgtctttatctggccatggatgagaacagcatctttgataatggactttatgatgaaaagcccttgacttttttcaaccctttgttttctggggctagctgtgttggccgcatggataggttgggctatcctcgctggaccaggggtcagaaccgagagggcgaggagttcatccgcaatgtcttccaccttgtcatgcctttattttcaggcaaagagaagtcccagctctgcttctcctggttacagtatgagattgcaaaggtcatttggtgcctgcacactaaaaacaagaagagattaaagtctcaagggaagaactgcaaaaaactagccaagaatctccttaaggagccagaaaactgcaacaacttttgcctgtggaagcagtatgcacatctggagtggttgcttggcaacacggaggatgccagaaaagtttttgacacagcacttggcatggcaggaagcagagaactgaaagactctgacctctgtgagctcagtctgctctatgctgagctggaggtggagctgtcgccagaagtgagaagggctgccacagctcgagctgttcacatattaaccaagctgactgagagcagcccctatgggccctacactggacaggtgttggctgttcacattttgaaagcgcgaaaggcttatgagcacgcactgcaggactgtttgggtgacagctgtgtctccaatccagctcccaccgattcctgtagccgcctaattagcctggctaaatgcttcatgctcttccagtatttgaccatagggattgatgctgctgtgcagatatacgaacaggtgtttgcaaaactgaacagttctgttttcccagaaggctctggcgagggggacagtgccagctcccagagttggaccagtgttctcgaagccatcacactgatgcacacgagcctgctgagattccacatgaaagtgagtgtttacccgctggcccctctgcgagaggcactctcacaggctttaaagttgtatccaggcaaccaggttctttggaggtcctatgtacagattcagaataagtcccacagtgccagcaaaaccaggagattttttgacacaatcaccaggtctgccaaacccttggagccttggttgtttgcaattgaagctgagaaactgaggaagagactggtggaaactgtccagaggttagacggtagagagatccacgccacaattcctgagaccggcttaatgcatcggatccaagccctgtttgaaaatgccatgcgcagcgacagtggcagccagtgccccttgctgtggaggatgtatttgaactttctggtttccttaggaaataaagaaagaagcaaaggtgtattctacaaagcacttcagagttgcccttgggcaaaggtgttgtacctggacgccgtggagtatttccccgatgagatgcaggagatcctggacctgatgactgagaaggagctccgggtgcgcctgccgctggaggagctggagctgctgctggaggattagagagcagtgggaaaacgggctgtgcctgcgaggccaagttgcccaccctgcggagctaggaggcgcgagcagagaacgtgtgtgttaggagaactcggcttttgaaatgttctttctcgatagtaataatgtgggctgccagcctctcacatcttgcacactttttgggtgtgtaaatgacacaaaagttatttacatattatatatgataatacgcgtgtatatgaca

SEQ ID NO: 48 - DNA, 1702 bp, Homo sapiens
  misc_feature: Incyte ID No 7040722CB1
ctccctgagcccacgggacgctgggaaccagccagccatggtgggaggggtcccacctgcgttggggtaggaggaggacacagtaccagttcccttcaaacactcaccggtgctggggttgtaaacatccccgtcgttcaccagcactttgttaaagaggacaacgcccccatcactggggaaaggcttctgggtgagccccgcagaaaaagacaccagagaaggcaccggagctcctggagcagagaaaggatggaatggattgacccgggtgcttctgtcctccagggggagggcctggaaaactgaggcctacgtgccaggcccaggtgccaggcccattttatttcacccaaagtggtgaagcaaactcctcatgcccaacagccagagggttccctgtctgagggatcagcaggcaggacagctgaggaatctgagaaaccggacagactcttgttaggagggtgtgtgtgaggctgggcatgtaagcatatgagatgagcatgtgtatgtgcgtgtgagtgtgtgcacacacttgtgagcacacatattaggaggccaggccaggcagtgggtgcatcatcatctagggagctgcttccttactaataaatcagtaaaacctggagccccaggaagctcatcaggaccacacaagggcagaggcccacccagtgatccccttgggaaactcaatgagctgggatgatggaggagggaagattccttgtcttgggcctgactctgaccctctctggggagcaaaccttggggaatttggtccactatagtcagcagagggctgtcccagtgctggtgggtggggaagggctgggcccaggcttgatttgtgctaatagcctggagggaggaggcaggagatgcagaggaggggcagaggccaccaaggagaccgctcctcctgctcctggtctctcagcagacatacctggggaagctacaggaggtgacttcgggtatcctgaaaaacagaaaggaaatggcatgtcaaacaagaccctaagcaccagctccttgttctgagccccctttttcctctgctcctcacaagttcaaatcgagtcttcactactatagaaacagacatagaacaagggcacccactgtcactgccctccttcaagcagaggcacatttgtaccagcccgtccctgcttcctcactagcccatttatcctggcggggaagagggcactgagggcaagctggctgcaggaaagaaagagatgggcactggagccttggagacttctctgtgactgagagagtcacacaagccaactaacatttgccctctagcagtgttatctgttcaagagctctaccttcaactagactgaatgcagtacttattaaatggaggcatggatgggtgggtgcagaagtggagggacaaatagatggatgagtggatgcacgcagggaagagtgcacggcttgatgtgtggaaggatgtgcacatgcataggtgggtggacagatatgtgcatggatacatgggtgggtgggtccctggggtgccccagcctctgcatatgaggatgcatatgtgggtggatggatgtatcgatgggtggatacacggatacatgtgtgggtgggtggctggatggaaggatggatggaagggatacacggatagatgcatgggtgagtgctgcatgcacggaagggtgcg

SEQ ID NO: 49 - DNA, 1462 bp, Homo sapiens
  misc_feature: Incyte ID No 6430290CB1
aagaccctgtctcaaaaaaaagaaaaaaatcagttgcagatgtccataccttatgatgattagccaaggactggtgacacacctcatggcagattgcacgtcagggtcgagagccagtggctgaggacagctctatgctcaggcttgtgtttccccagcgtgttgtgccctctgcagccctcttgtgcccagacacagagtagttaaaaacagcagtacagagggcaagacatggaactgcctacttttttgctcagtgggaaaagtgcttgggtgagacatgtggctgtccattgaagggaacagctatgctgactgtgggttggaaaatctaggctggctgtcttgctggctgtcccgtgttggctggtttggtgcctacagtggagtcccttgtctgcctgtgtgtctcttaatgattctgatgtcatctctgcgaggtcagaggttggttcccggggcaggcccatcttgcaccctgaggttcagatgggaccacaagcagcgtagcacagggggaaaacacttgtgcgcgtcgaccttgcacatcaacaagttagaccagaacatggagtgcaccagccttctcagaattcttgagctctctgtaccaccactaggggctcctctgtggctcaagacccctcctgaggcagagggtgaggaggaggcagaaagaaaagagaacaggagacttggagtgggcagcagtagagcggaacccagcaggcccgcctttttggattcctctgggggctctggggatgtgtgttgtgggagtggagagtggtgacgagcagaggacggacagctgctgaaagcgatcattcctctccttgtggggcatcctgggaagaacatgggtgatctctgcagtcaggcagcgcagtcctcgtggggccttatggggcggctctgggtgtctgcattctaatccctggcaacaaatggggctgcaaggctgggctcctggtagatgctcaggaagggttgttccactcctcaccccctcctgagctgctttctagcctcgcagccccgtctttggatccctttttggcactctgctgggctcagagcttccgtggcaggagcagaggctgctctctcctgggttcctctgggagctaccatggagcttgggcagttccttctattgttgcgattctgggttcaaagccacaggcctctcagacagctgtgacttcccggcaggcagtagggatggggtgggggtggaggcgttggggccctgcagcttccctcgcttgcctctgtccttgctaggcagtgagttggaggcgcctgggctcagctgatgttctccaaggaggacttggaatgaacccgagacacaaagggtgaggtcagtggtttgctcagcaagaaggctgagttagtgacaacctgtcacagctcccaggatccctggctgcagtccctggctccgaatcctccccct

SEQ ID NO: 50 - DNA, 3958 bp, Homo sapiens
  misc_feature: Incyte ID No 2640251CB1
tttttttttttttttttctaaagtttcagtcagttttattacatccagtccagtaagctgtgctgtctagtggtatccacacatactttattagttttatctttgttcagcccaccaacaaaaatttggaaagattgctacacatgacagagcgacagtaccctagggtgacatgacagcccatatatcagttctctgtttcacattaaaaagttaccattcattcggtccaaacatccacccaatccacacgttctctcaaagactagcatggttctgtccaataccttgctctaattccaattcattcgttctcataacttatttgaaaaggcacatttggtcagtctgatgtcattcttagggcatctttgctctctcctggccattactgcttgcttttatgtactgaaaagtcatccattgaatggtctgttcctgaatgtcgccacaaagcagtctgtaaaacccactcactttccctccttaaattacctccctccaggtctgtttcctcaaagatcgtcccttagtgggtcacagtaatcatgaaccctgggtacaattgatacaggctgagaagtaagaattcactgttcacttagagcaactcattccctgacctgaaccgggtgtgttccacccttgcccatggttagatctctttccttgatagaacaagcaaaagaggggctgatccatctcccgtgaacatctgttgactgtgggcggcctgggccagccttgctgtgtttgcctcagatgcgagtaaggaagctccagttgtagccttggcacagctgaggccatgccattgctacccggaagctggctctgcagcgttgtgacttagacactttacaagtctgagccatcccgtactagttcatcaactttccttcctcatgatctgcaaagatacagagagaatggcattccaaaattccatggatgacttttctgaatgtcctagaatgtttggcaaagatctggctaaaactagtactatggaatagaaaaggaaaagtgacatttgtgtaatttattttggaaatgcaaaggaattattatgcagcaatcttaactttgtgtacataccactcacctcccttaaaattcagcagaaccagaggtgccagaaatgtcaaaaaatatctagtcattccaatatgtaaaacaagactgccttgttttaaaaaaacaaattaaatgtaaaaattgtatgactcttacccagtgaaaagctttactaacacagccctgggtgctatttccagatactgaaaagatctaagtaggtgcttaagaactgctttttaacccattcacaaccttcatattaaactaggatgaggctgcaggagctgaggaaggaaggcctgggtgggaacctggatgtcctttcctggagaacagctttgctatttcactggtaactccgaaacttcaaaatattgactgtacaaattactcaagtttattttggcctaattattcttaagaaaggtggttcttttttagaaaagggaagcagaagctaattgaaattccattactcaggcctctagtaagtgcccccttcatgtagtcactattcactgagggccatgagggtgaggccctgctgggggggcagggcagggctgtgagctcaggagcccatccctgtgctcagagagatgggactccaatgaggagacacactatgtaggtgcaatttctgatttcttagtttttgaccaaaatagcagtaatttcaaacagttgaatgtcgtgatcgatactatgtcagttggtgatgaatgctgtgaatggtattgaatgtcctggaccaagaggaacagcaacatgtccttagtcctcagcctaagaagtccatcgagtccttctctcaaattgtacgtctgttgttttcaaaagccattgtgacaggtcaaaaccctggagtatttggtttgtgtttaaatgtacttgacactctggaaacactcaacctaagccctggatgcaggaggcagggatgctgaggatgtgaattagagtgctgggatgtgctaatgcctagggaggcatcaggcactgtgctggcttgggggatcctgtgatgactggaacctgggccttcattacgggctccggttttggagtgcagagggattggcatttgtaaagactgcttgcttaccctttgtctggcatgcacattcaggatgtgacttcagcttcattcatcctggccaaacaaaaaaagttcaaaaaattgttttcaattaaatttctgaagatctttgtagggaagcacccttctgagatgaaaccaatttcttgtccacacagccaccatatccttatatagatggggaaaaatccagaaaagggggattttcagcagctggcttgaagcccctgaatggtcccttggcagaggcaaagaatgagcccagagcctgagattcaagatgctggtctcttctgaacacaccgtggagcaatgtggtgcacctgcagatgccgcactcagcagcctggaatgccagctctcatgccctcacctagaaacgccttctcggctgaatggctgttggctctcagcagcacctgtcagcatagctcggccgcttctgccccgtgccaaggcgtgactaggaccagaagaatcagaaagcatggtggagtccgaaagcattggcaaagccagcacatctgtgagcatgttacacatgggcttgcctaagtatttccttctctgtgaacatcacttaggagctacgtcagttaaggcatcgtgagacacaggtgtggtggaggcattatgaggggactactccacaggaccttggagagttgcattttggggggctctcctagaagctctgggggaaggaagcagtggcctcctggtcagctacagacacaggcgggttagggcccgtcttgctttcatgttcttttaaacacaaatggcagctcacaaatattcctctaaatattcctccatacagcctagggcccctcaagagacgtgcaaaccaaagtatttgtttttctcactttatctttaacctagtatgtggcaagtctggaccactgctttagagaaagctggattaaatctcttgagcagaagtagagaccctagagaatcttcactggataccttgcttcacactttggtcaatatttatgaataagttctaaagcctatgcgaagcccattctgttttgcttggggaccagccagaccactcctccactccagtggggccttttttgtgtctttcacatcactgttacaagttgtggctggacaattaatgacatttaaatcactgtcccacatatggatatattagaaaaaccctaatgttttaaaatttttttaatcatcagctgcttttacaaagctttgcatgcatttgtttagaggatattgcagtcgagatattccagtactactgtgtgatcatatgaaaaaagttggttttctctctgtcaaagtcaatagggtggtagactaactcacagagtgccttacttcttcctcccgattaatcgtgttcactatatggtggcagaatcagtccagtcccccagacctcaagctgcatcttccaaatggttggtgtctttttaagagtccccgtgggctggctttcctggaagaagtccattaaattccctgcttctataagctgcaggagtcgcctaggatatggcccgtcacgtctggggatgaccctgcactgctctccagatttcactcaaggttcgtatttttctctgccaccttgaactacagaacaggctctcagtagcgtgataagctttaagctagaatgagatggaaaggtgggaaagacagacaggaagtgtagctcttagtctatggggctctccttaggggacaaaaagatggcttaggctccgtgggttttcaactcgtagaaaaaataacctg

SEQ ID NO: 51 - DNA, 826 bp, Homo sapiens
  misc_feature: Incyte ID No 3839350CB1
gattctttccactgaggaggcaaggatggagttgctgctgacccgtgcagatttgctgctggtaacatactcatgggcaaagtctgaaaaacctcttcctgccctgggcaaggcagatggggcagcccagcaggctggctttgcccaccgtctccgatatcatccaacacagtcagccaggttgtgtggccctctcatctacattctcatttgccaacaagatggtgtgggctatgctgtctttgcacagcgatctgatcccagggacatttgtgtgggtgtgtgcccgggagctgcatgtctgcaggttcagattggggtcagctttgctggtgagtgtggcttcctgccatggcaccaaagaagagggctggcggattagtcctgttgtctctcatagctttgtagggacacagcgggccatgtggactgacagctggagacttcataaagatggcctagaaaattaggacactgtcctgttgacaaagcccctaggcctgcccccatcaggcctcatgggtccttttcaggcctaagccagggttctgaaatcatctgtaatgtgaatttatatttctaggccacattcgctgcatcctttccagcaagatgtcattctgagatttttgtcattcctgggcgttatgaggactgccaagggacacaattccatgggaggaaagttttcttctaagctgcaagccagctctggcttctggagtttctccttaggattgcaagacagtagttccctgtcctttaggtcttctccagaaataccttaaaatagcaaataaaataagatataagcaaaaaaaaaaaa

SEQ ID NO: 52 - DNA, 729 bp, Homo sapiens
  misc_feature: Incyte ID No 6393813CB1
aagaattcggaacgaggccggcggggggtcaggatcctccacaggtaggcgcagtcagctggagcgtcgcggcggtccgccggtcgtggagggcgtgtcctgcggcgcgatggccgtagtgttgccggcggttgtggaggagctcctgagcgagatggcggcggcggtgcaggagagcgcgcgaattcctgatgaatatctgttatcgctgaagtttctctttggctcatcagccacccaggccttggacctagttgatcgacagtccatcaccttaatctcatcacccagtggaaggcgtgtttaccaggtccttggaagttccagtaaaacatacacatgtttggcttcttgtcattactgttcatgtcctgcatttgcattctcagtgctacggaagagtgacagcatcctgtgcaagcatctcttggcagtttacctgagtcaggttatgaggacctgtcagcagctaagtgtctctgacaagcagttgactgacatattattgatggagaagaaacaagaagcataaaaggactgcaggaggtgctgtgggttggagccgtgggctgtggagggtttgtgtatgatgagaagccctgtacagtcttgtcaagaaataccctgagccagtctctgagacgcttcggtaaaaaatgtccctggatggaatcaagattttaaattcaaataaagcctaatatcatgttgtgtccacaaaaaaaaaaa

SEQ ID NO: 53 - DNA, 1610 bp, Homo sapiens
  misc_feature: Incyte ID No 5685755CB1
tgccaacagaaagggggtcccttccctgtgccaggatcctatataaggaagagggatgtgagcagagtggtctggcttcaccgtccctccagtggacttgggggctgagcataaagatgcctaggagaggctactccaagcctgggtcctggggcagcttctgggccatgctgaccttggtgggcctggtcacccatgcagcacagagagccgatgttggcggggaggcagctggcacctccatcaaccactcccaggcggtgctccagcgcttgcaggagctgctgcggcagggcaacgccagcgatgtggttctgcgggtgcaggctgcgggcaccgatgaggtccgggtattccacgcccaccgcctgctgctgggactgcacagtgagctgttcctggagctgctaagtaaccagagcgaggcggtgctgcaggagccacaggactgcgccgctgtcttcgacaagttcatcaggtacctgtactgcggggagctgaccgtgctgctgacccaggccatccccctgcacagactggccaccaagtacggcgtgtcctccctgcagcgcggcgtggccgactacatgcgcgcgcacctggcgggaggcgcgggcccggcggtgggctggtaccactacgcggtgggcaccggggacgaggccctgcgcgagagctgcctgcagttcctggcctggaacctgtcggccgtggcggccagcaccgagtggggcgccgtgagccccgagctgctctggcagctcctgcaacgctcggacctggtgctgcaggatgaactggagctgttccacgcgctggaggcctggctgggtcgcgcgcggccgccccctgccgtggccgagcgggcgctgcgcgccatacgctaccccatgatcccaccggcacagctgttccagctgcaggcgcgctcggcagccctggcgcgccacggccccgcggtggccgacctcctgctgcaggcctaccagttccacgccgcgtcgccgctgcactacgccaagttcttcgacgtcaacggcagcgccttcctgccccgcaactacctcgcgcccgcctggggcgccccgtgggtcatcaacaacccggcccgcgacgaccgcagcaccagcttccagacgcagctgggcccgagtggccacgacgcgggccgccgggtcacctggaacgtgctcttctcgccgcgctggctgcccgtcagcctgcggcccgtttacgcggacgccgcgggcactgctctgcccgccgcgcgcccggaggacggccgaccgcggctggtggtgacgccggccagcagcggcggcgacgcggcgggcgtgagcttccagaagacggtgctggtgggggcgcgccagcagggccgcctgctggtccgccacgcctacagcttccaccagagcagcgaggaggccggcgacttcctggcgcacgccgacctgcagcggcgcaactccgagtacctggttgagaacgccctgcacctgcacctcatcgtcaagcccgtataccacacccttatccggacccccaagtagcctcggggtctgggaataaaggcctggcctaggtggtccctgtgggtctgggaaggg

SEQ ID NO: 54 - DNA, 869 bp, Homo sapiens
  misc_feature: Incyte ID No 71728459CB1
ctctaggacatccctgcagaaaggagagaagcaaggagaaggagtgacaggtgttttgtcaggcctgtatcttcagccagatacaaagcccccacatgtacacctgctctcattatgtctcattcgtgagcagttaatcaagttaattatgagcagttgagccctgggagcttttactgacagtgcaacctataatttctctgaaagttacagagccagagatgaaggttactgctcttttaggagccctgtctccagtttttgcttttgtttctgtttttatttcatggttagcatcctttggagaccagaagagcattgacagccccccagatgagcagcagagcaattcttatactagtggtcaagcagcttcctacagccaaaaggctataggaagaaagggtaactggctgccctacagtttacacgatgaagctgccttgggctctgggtcttggtgatttagccagaagattgtttctcagagctcagccctacacattctcctaaaggccttcttcctggcataggtcaaagacagaacctgagagagggaaatgagccagaacaaatatgtcatctgatgacctcacccccagcaaatgggcagggaagagagctagaatgagaaggccagaacatggagcaccatgaaaagaaactgctcaagcaagagaagtggcatgtgggctttaaagaccagggttagaaaacagcagccctgacctttgtgttgaagtagcggggcgaggatctctcatagaagtgttaattctgtactccttccccgattctgagtaggtgccttttacatagagatgatcaatgtgtacttgtctaattaattacaaactgcccccagtgta

SEQ ID NO: 55 - DNA, 2209 bp, Homo sapiens
  misc_feature: Incyte ID No 1904303CB1
gcctgtgcgctgtgcctccgcttgtctgctctcccccgccttggccttctctcgccgccccctttcgcgtcctctgtccttgtgtcctctctctcccgtctcgctctcgtgtccttggactgctccttctgtctcgcgcgctccttggtcgctccctccgtgttgctctgttgtgtggttagctgtcttccttttctcttgttccgtcgctctttgcttcttttcctttggttcgttgtgtctgtgcctgtctctggctttcttggtttttgtgcccaggcccccacgttggccgctgctgccgggctcaccccagccccgcccggaggcgccccgcggccccggctagccagggcgggcggccacactctgccctactccttccctccgcgttccgggcctcggagccgccttgaggaggatgagtccctggagctggttcctgctgcagaccctctgcctcctgcccacgggcgcagcttcgcggcgcggggcgcccggcaccgccaactgcgagctcaagccccaacaaagcgagctgaattccttcttgtggaccattaagcgagacccaccatcttacttctttggcacaatccatgtcccgtacacccgagtttgggacttcatccccgacaactctaaggaggctttcctgcagagcagcattgtgtactttgagttggatctcacagacccctataccatctcagctctcaccagctgtcagatgctgccacagggcgagaacctccaagatgtgctccccagggacatctactgccgcctcaagcgccacctggagtatgtcaagctcatgatgcccttgtggatgaccccagaccagcgcggcaaggggctctacgcagactacctcttcaatgctattgccggaaactgggagcgcaagaggcctgtctgggtgatgctcatggtcaactccctgactgaagtggacattaagtcccgtggagtgcctgtcttagacctgttccttgcccaggaggctgagcggctgaggaaacagactggggcagtggaaaaggtggaagagcagtgccatccattgaatgggttgaacttttcacaggtcatctttgctttgaaccagaccctcctgcagcaggaaagcctgcgagcaggcagtcttcagatcccctacacgacggaggatctcatcaaacactataactgcggggacctcagctccgtcatcctcagccatgacagctcccaggttcccaattttattaatgccacgctaccacctcaggagcgcatcactgctcaggagattgacagctacttacgccgggagctgatctacaagcggaatgagagaatagggaagcgggtgaaggcccttttggaggagttccctgacaaaggcttcttctttgcctttggagctggtcatttcatgggcaacaacacagtgctggatgttttgcggcgtgaaggctatgaggtagaacacgcccctgctggacgacccatccacaaagggaagagtaaaaagacctccacacggcccactctgtccaccatctttgctccaaaagtccctaccctggaagtaccggcaccagaagccgtatcctcagggcactcaacgctgcctccccttgtgtcccggcctggaagtgccgacacgcccagtgaggccgaacagaggttccggaagaagcggaggcggtcacagcggaggccgcgactccggcaattcagcgatctgtgggtccgcctggaggagagtgacatagtcccgcaactccaggtccctgtcctggacaggcacatctccactgaactgcggctccctcgccgtgggcattcccaccacagccagatggtggccagcagtgcctgcctgtctctctggactcctgtgttctgggtgctggtgctggctttccaaacagagacacccctcctgtaacgactggaagcaccaggctaagaacctgacccctcggacttgaagaatggccattcctgtactccacattctggtctagccttgttgggcccaatccagaagagactgctcttgaaaaagcggcccagtgttgattcttctctttccaaggaatgtgactttgggttatccaactttgggggcaggtgtacagttttgtaacatagtgagttgtgtgaaaataaattataaatgagttgtatgaaaataaaaaaaaaaaaaaaaaaaaaa

SEQ ID NO: 56 - DNA, 1520 bp, Homo sapiens
  misc_feature: Incyte ID No 2911343CB1
ggccaacggagcccgcctttacctccccactcggcgtccagtctcttagcaacgactccggcttcctaggaactgctcctttctcaaccattcctgcccacaacaccccagcttgctgccagcaaagcccctccacacccctcaaactccagacccttcacatcaatttactgttttcttcgacctcaccccagcaattttctctgaatcaaccccctttcctcctctcctaagtctctgagtttctctctcctcctgccatcctgagttctgccatccttaggggccgccaagacctctcttttcgttcctctcccgcctcagaccagcagccttttattttagatcatgtctggagggaagtcagcccagggtccagaggaagggggcgtctgcatcactgaagcccttatcactaagcggaacttgactttccctgaagatggggaactgtcagagaagatgtttcacactcttgatgaactgcagactgtccgcctggaccgggaggggattactactatcaggaacttagaaggcctccagaatcttcacagtctctatctgcaagggaataagatccagcaaattgagaacctggcttgcatcccctccttgcgcttcctgtctctggcaggaaaccaaatcaggcaggtggaaaacctcctcgacctcccatgcctccagtttctggacctttctgagaacctgatagaaacattgaagctggatgagttcccccagagccttctcatcctcaacctgtctggaaacagctgcaccaaccaggatggctaccgcgagctggtgacagaagccctgccacttctcctggacctggacgggcagcctgtggtggagcgctggatttcggatgaggaggatgaagcctcaagcgatgaggagttcccagagctgagtggcccattctgctcagaacgaggcttcctcaaggagctggagcaggagctgagcaggcacagggagcaccggcaacagacggccctgacagagcacctgctgaggatggagatgcagcccaccctcaccgacctgcccctgctacctggggtgcccatggctggggacagcagcccttctgccactcctgcgcaaggggaggagacagtccctgaggccgtctcctcaccccaggcctcctctcccaccaagaaaccatgcagtctgattcccaggggccaccaaagctctttctggggaaggaagggggcacgagcagccacagcccccaaggcctctgtggctgaggcccccagcacaaccaaaactacggccaagagaagcaagaaatgattctctgtcaacctttctctactagtggagaggagtggggcctgcccctcttctcagacctctgacctgtgacagaagcccatccccagtaaagtgtctctaggccctgagtatgcttttcatgtcacttggggtatctcaggggaagaaaccagtgaaagctccaggaaacaaaatacagagctcagtgagccacctgaaaaaaa

SEQ ID NO: 57 - DNA, 1282 bp, Homo sapiens
  misc_feature: Incyte ID No 7500308CB1
agcccctgcagcagggtgggagcattaggacatgtccacacgtcttcaagggaaaatggctaagggctgtcagagaggcccagggaagaggcctgggtaggggcgagcagggagcagacaatcacttgttgaaggaagatccaagtccaggaaggaacgtagggcagtttggtgtcatggaaggaactgggggcccagccgaggggctcaagagcccttcagactctgcctgagcgagggtggtgctctgtcacccaggctggagtgtagtggtgtgatctcagctcactgcagccttgaactcctaggctcaagtgatcctcctacctcaacctccagagtaactgggactacaggaaagctcagtggcccccaagccaggatgtcccaagcttgggtccccggcctcgcgcccaccttgctgttcagcctgctggctggcccccaaaaggtgattctgaagcccagcctgggcccaactcccacagagccaccccctccctacagcttcaggcctgaagaatataccggggatcagaggggcattgacaacccggccttctgagtcacctcctgcctggaatcttgccatcagcaacctcctccccagtgcctcctggatcaagctagagactgctggcaccccaggaatgtccctgcccatcctgccgtgtctctgttcattcttggatttaacttattactttttctgcttctgtttccaccccagctgcctctcttgtcctgagggttaggctggagtgacagtttccgcccaccccccagcccaagaaagaggctgccggaaagaaaatgctgaccattggaggtgcccaacagtagaatgggctactgtgaggggtagtaagagccccatttctggaggtatgcgaatcttgactggacagccagctctgagattttatcagggcacttctatacctgtgggacattggactggatgagccctgagccagcttccactcctacctgaatagagaactcactgcacccacccacaacacatgataaacacatgtcctcactgaatgttactgattgcggctgagggcctgcctctggctgtgtggggaggtgggtggagaggtgagcccaggcactgctgaggggtgcggtgatggggtcgctgcgccgcaatcccaccactgatgagccacctgggaggtctgggaggccagtccatccatgggccgccctcggagagaggcttgttctagatgtattggctgtctgttttttgatgtctctgtgtgccaaacag

SEQ ID NO: 58 - DNA, 1228 bp, Homo sapiens
  misc_feature: Incyte ID No 7501098CB1
gcgaaccccaggcccttcccaggtttgcgcgcgggggccatccagaccctgcggagagcgaggcccggagcgtcgccgaggtttgagggcgccggagaccgagggcctggcggccgaaggaaccgccccaagaagagcctctggcccgggggctgctggaacatgtgcggggggacacagtttgtttgacagttgccagactatgtttacgcttctggttctactcagccaactgcccacagttaccctggggtttcctcattgcgcaagaggtccaaaggcttctaagcatgcgggagaagaagatttgatctggagctcttcactcccggcaacctagaaagagagtgcaatgaagaactttgcaattatgaggaagccagagagatttttgtggatgaagataaaacgattgcattttggcaggaatattcagctaaaggaccaaccacaaaatcagctcttcagccgtctatgaaagggggaggcacactccctccatcattttcagaagacctgaggaggctgccttgtctccattgccgccttctgtggaggatgcaggattaccttcttatgaacaggcagtggcgctgaccagaaaacacagtgtttcaccaccaccaccatatcctgggcacacaaaaggatttagggtatttaaaaaatctatgtctctcccatctcactgactaccttgtcattttggtataagaaatttgtgttatttgataggccgggcatggtggctcatgcctgtaatcccagcactttgggaggccaggagttcgagaccagcctggccaacatggtgaaacccggtctctactaaaaattcaaaaattacctaggcgtcatggggcatgcctgtagtcccacctacttgggaggctgaagcaggagaattgctcgaacctgggaggcagaggttgcagtaagctgagatcacgccactgcattccagcctgggcgacagagcaagactccatctcaaaaataaaataaaaaaagaaaaaaagaaaagaagaagaaaagagaagaaggagaaggagatgaaggaggaggaggaggagaaggagaagaagaagaagaagaagaccacaaaagacatggactatccaacttttatgacaactgcaggaataaggagaatagtcatgtactgtacacgaagtctgtctgcatctggactgaactgatcatcatcagtgatagagactttgatcta

SEQ ID NO: 59 - DNA, 3582 bp, Homo sapiens
  misc_feature: Incyte ID No 7503839CB1
gcctctccctgccccgccgcgccccagtcccttgacgaccctcctctctgggccccgcccctcccgcttcggggtcaagccccagagagcgccgcgaaaaccacatttcccagagtgcaccgcgacggcaggggtcctcagaccggcgctcgctcgccggcgccatccctatagagaagaacggaggtacggcctgtggtcatggcgctgttcccagcctttgcggggcttagtgaggctcccgatggcgggagctccaggaaagtcaaggaaataatgctgcagctgatactggacatcgctttgtttggcttgaggacattcaggctgtgacgggagaaaccttcagaacagataagaaaccagatcctgcgaactgggagtacaagtctctctaccgaggggatatagcaagatacaagaggaaaggagactcctgccttggcattaaccctaagaagcagtgcatatcttgggaagggacttccacagagaagaagcattcacgcaagcaggttgaacgctattttactaagaagagtgtgggattaatgaacatcgatggagttgccattagcagtaaaactgaacctccctcatctgagcccatctcctttataccagtgaaggacttggaagatgcggctcctgttacaacctggttgaatcctctggggatttatgatcagtcaaccacacattggctacaaggacagggtcctccagagcaggaatcaaagcagccagacgcacagccagacagcgagagtgcggctctcaaggccaaggtggaggagtttaacaggagggtgcgggagaatcctcgggatacgcagctgtggatggcatttgttgcttttcaggacgaggtcatgaaaagtcctggcctgtatgccatcgaggaaggagagcaggaaaagcgaaagaggtccctgaagctcattctggagaagaagctggccattctggagcgggccattgagagcaaccagagcagtgtggatctgaaactggccaagctgaagctctgcacagagttctgggagccctccactctggtcaaagagtggcagaaactgatatttttgcatcccaataatacagccctttggcagaaataccttttattttgccagagccagtttagtaccttttcgatatcaaaaattcacagtctttatggaaaatgcttgagcactttgtctgctgttaaggacggcagcatcttatctcaccctgcgttgcctggcacggaagaggccatgtttgcactctttcttcagcagtgccactttctgcggcaggctggccactctgagaaggccatctcattgttccaggccatggtggacttcaccttcttcaaacccgacagcgtaaaagatctgcctaccaaaggacaggtggaattctttgaacccttttgggacagtggagagccccgggctggggagaagggagcccgaggctggaaggcgtggatgcaccagcaggaacgaggtggctgggtggtcatcaacccagatgaggatgacgatgaaccagaagaggatgaccaggaaataaaagataagactctgcccaggtggcagatctggcttgctgctgagcgttcccgtgaccagaggcactggcggccctggcgccctgataagaccaagaagcaaaccgaggaagactgtgaggatcccgagagacaggtgttgtttgatgatattgggcaatctttgatcagactttccagccatgatcttcagttccagctggtggaggccttcctgcagttcttgggtgtgccttctggctttactcctccagcctcctgtctttatctggccatggatgagaacagcatctttgataatggactttatgatgaaaagcccttgacttttttcaaccctttgttttctggggctagctgtgttggccgcatggataggttgggctatcctcgctggaccaggggtcagaaccgagagggcgaggagttcatccgcaatgtcttccaccttgtcatgcctttattttcaggcaaagagaagtcccagctctgcttctcctggttacagtatgagattgcaaaggtcatttggtgcctgcacactaaaaacaagaagagattaaagtctcaagggaagaactgcaaaaaactagccaagaatctccttaaggagccagaaaactgcaacaacttttgcctgtggaagcagtatgcacatctggagtggttgcttggcaacacggaggatgccagaaaagtttttgacacagcacttggcatggcaggaagcagagaactgaaagactctgacctctgtgagctcagtctgctctatgctgagctggaggtggagctgtcgccagaagtgagaagggctgccacagctcgagctgttcacatattaaccaagctgactgagagcagcccctatgggccctacactggacaggtgttggctgttcacattttgaaagcgcgaaaggcttatgagcacgcactgcaggactgtttgggtgacagctgtgtctccaatccagctcccaccgattcctgtagccgcctaattagcctggctaaatgcttcatgctcttccagtatttgaccatagggattgatgctgctgtgcagatatacgaacaggtgtttgcaaaactgaacagttctgttttcccagaaggctctggcgagggggacagtgccagctcccagagttggaccagtgttctcgaagccatcacaccgatgcacacgagcctgctgagattccacatgaaagtgagtgtttacccgctggcccctctgcgagaggcactctcacaggctttaaagttgtatccaggcaaccaggttctttggaggtcctatgtacagattcagaataagtcccacagtgccagcaaaaccaggagattttttgacacaatcaccaggtctgccaaacccttggagccttggttgtttgcaattgaagctgagaaactgaggaagagactggtggaaactgtccagaggttagacggtagagagatccacgccacaattcctgagaccggcttaatgcatcggatccaagccctgtttgaaaatgccatgcgcagcgacagtggcagccagtgccccttgctgtggaggatgtatttgaactttctggtttccttaggaaataaagaaagaagcaaaggtgtattctacaaagcacttcagagttgcccttgggcaaaggtgttgtacctggacgccgtggagtatttccccgatgagatgcaggagatcctggacctgatgactgagaaggagctccgggtgcgcctgccgctggaggagctggagctgctgctggaggattagagagcagtgggaaaacgggctgtgcctgcgaggccaagttgcccaccctgcggagctaggaggcgcgagcagagaacgtgtgtgttaggagaactcggcttttgaaatgttctttctcgatagtaataatgtgggctgccagcctctcacatcttgcacactttttgggtgtgtaaatgacacaaaagttatttacatattatatatgataatacgcgtgtatatgaca

SEQ ID NO: 60 - DNA, 2063 bp, Homo sapiens
  misc_feature: Incyte ID No 7503698CB1
gcctgtgcgctgtgcctccgcttgtctgctctcccccgccttggccttctctcgccgccccctttcgcgtcctctgtccttgtgtcctctctctcccgtctcgctctcgtgtccttggactgctccttctgtctcgcgcgctccttggtcgctccctccgtgttgctctgttgtgtggttagctgtcttccttttctcttgttccgtcgctctttgcttcttttcctttggttcgttgtgtctgtgcctgtctctggctttcttggtttttgtgcccaggcccccacgttggccgctgctgccgggctcaccccagccccgcccggaggcgccccgcggccccggctagccagggcgggcggccacactctgccctactccttccctccgcgttccgggcctcggagccgccttgaggaggatgagtccctggagctggttcctgctgcagaccctctgcctcctgcccacgggcgcagcttcgcggcgcggggcgcccggcaccgccaactgcgagctcaagccccaacaaagcgagctgaattccttcttgtggaccattaagcgagacccaccatcttacttctttggcacaatccatgtcccgtacacccgagtttgggacttcatccccgacaactctaaggaggctttcctgcagagcagcattgtgtactttgagttggatctcacagacccctataccatctcagctctcaccagctgtcagatgctgccacagggcgagaacctccaagatgtgctccccagggacatctactgccgcctcaagcgccacctggagtatgtcaagctcatgatgcccttgtggatgaccccagaccagcgcggcaaggggctctacgcagactacctcttcaatgctattgccggaaactgggagcgcaagaggcctgtctgggtgatgctcatggtcaactccctgactgaagtggacattaagtcccgtggagtgcctgtcttagacctgttccttgcccaggaggctgagcggctgaggaaacagactggggcagtggaaaaggtggaagagcagtgccatccattgaatgggttgaacttttcacaggttcccaattttattaatgccacgctaccacctcaggagcgcatcactgctcaggagattgacagctacttacgccgggagctgatctacaagcggaatgagagaatagggaagcgggtgaaggcccttttggaggagttccctgacaaaggcttcttctttgcctttggagctggtcatttcatgggcaacaacacagtgctggatgttttgcggcgtgaaggctatgaggtagaacacgcccctgctggacgacccatccacaaagggaagagtaaaaagacctccacacggcccactctgtccaccatctttgctccaaaagtccctaccctggaagtaccggcaccagaagccgtatcctcagggcactcaacgctgcctccccttgtgtcccggcctggaagtgccgacacgcccagtgaggccgaacagaggttccggaagaagcggaggcggtcacagcggaggccgcgactccggcaattcagcgatctgtgggtccgcctggaggagagtgacatagtcccgcaactccaggtccctgtcctggacaggcacatctccactgaactgcggctccctcgccgtgggcattcccaccacagccagatggtggccagcagtgcctgcctgtctctctggactcctgtgttctgggtgctggtgctggctttccaaacagagacacccctcctgtaacgactggaagcaccaggctaagaacctgacccctcggacttgaagaatggccattcctgtactccacattctggtctagccttgttgggcccaatccagaagagactgctcttgaaaaagcggcccagtgttgattcttctctttccaaggaatgtgactttgggttatccaactttgggggcaggtgtacagttttgtaacatagtgagttgtgtgaaaataaattataaatgagttgtatgaaaataaatacctttttttgtataaaaa